SEQUENCE LISTING (55 sequences)

SEQ ID NO: 1 - protein, 262 aa, Triticum aestivum
MKTFLILALLAIVATTATTAVRVPVPQLQPQNPSQQQPQEQVPLVQQQQFLGQQQPFPPQQPYPQPQPFPSQQPYLQLQPFLQPQLPYSQPQPFRPQQPYPQPQPQYSQPQQPISQQQQQQQQQQQQQQQQQQQIIQQILQQQLIPCMDVVLQQHNIVHGKSQVLQQSTYQLLQELCCQHLWQIPEQSQCQAIHNVVHAIILHQQQKQQQQPSSQVSFQQPLQQYPLGQGSFRPSQQNPQAQGSVQPQQLPQFEEIRNLARK

SEQ ID NO: 2 - protein, 291 aa, Triticum aestivum
MKTFPILALLAIVATTATTAVRVPVPQLQLQNPSQQQPQEQVPLVQEQQFQGQQQPFPPQQPYPQPQPFPSQQPYLQLQPFPQPQLPYPQPQPFRPQQPYPQPQPQYSQPQQPISQQQQQQQQQQQQQQQILQQILQQQLIPCRDVVLQQHNIAHGSSQVLQESTYQLVQQLCCQQLWQIPEQSRCQAIHNVVHAIILHQQHHHHQQQQQQQQQQPLSQVSFQQPQQQYPSGQGFFQPSQQNPQAQGSFQPQQLPQFEEIRNLALQTLPAMCNVYIPPYCTIAPFGIFGTN

SEQ ID NO: 3 - protein, 282 aa, Triticum aestivum
MKTFLILALLAIVATTATSAVRVPVPQLQPQNPSQQQPQEQVPLMQQQQQFPGQQEQFPPQQPYPHQQPFPSQQPYPQPQPFPPQLPYPQTQPFPPQQPYPQPQPQYPQPQQPISQQQAQQQQQQQQTLQQILQQQLIPCRDVVLQQHNIAHASSQVLQQSSYQQLQQLCCQQLFQIPEQSRCQAIHNVVHAIILHHHQQQQQQPSSQVSYQQPQEQYPSGQVSFQSSQQNPQAQGSVQPQQLPQFQEIRNLALQTLPAMCNVYIPPYCSTTIAPFGIFGTN

SEQ ID NO: 4 - protein, 297 aa, Triticum aestivum
MKTFLILALRAIVATTATIAVRVPVPQLQPQNPSQQQPQKQVPLVQQQQFPGQQQPFPPQQPYPQQQPFPSQQPYMQLQPFPQPQLPYPQPQLPYPQPQPFRPQQSYPQPQPQYSQPQQPISQQQQQQQQQQQQQQQILQQILQQQLIPCRDVVLQQHSIAHGSSQVLQQSTYQLVQQFCCQQLWQIPEQSRCQAIHNVVHAIILHQQQQQQQQQQQQQQQPLSQVCFQQSQQQYPSGQGSFQPSQQNPQAQGSVQPQQLPQFEEIRNLALETLPAMCNVYIPPYCTIAPVGIFGTN

SEQ ID NO: 5 - protein, 319 aa, Triticum aestivum
MKTFLILALLAIVATTATTAVRVPVPQLQPQNPSQQQPQEQVPLVQQQQFPGQQQQFPPQQPYPQPQPFPSQQPYLQLQPFPQPQPFPPQLPYPQPQSFPPQQPYPQQQPQYLQPQQPISQQQAQQQQQQQQQQQQQQQILQQILQQQLIPCRDVVLQQHNIAHASSQVLQQSTYQLLQQLCCQQLLQIPEQSQCQAIHNVAHAIIMHQQQQQQQEQKQQLQQQQQQQQQLQQQQQQQQQQPSSQVSFQQPQQQYPSSQVSFQPSQLNPQAQGSVQPQQLPQFAEIRNLALQTLPAMCNVYIPPHCSTTIAPFGISGTN

SEQ ID NO: 6 - protein, 660 aa, Triticum aestivum
MAKRLVLFAAVVIALVALTTAEGEASRQLQCERELQESSLEACRQVVDQQLAGRLPWSTGLQMRCCQQLRDVSAKCRSVAVSQVARQYEQTVVPPKGGSFYPGETTPLQQLQQGIFWGTSSQTVQGYYPSVTSPRQGSYYPGQASPQQPGQGQQPGKWQEPGQGQQWYYPTSLQQPGQGQQIGKGKQGYYPTSLQQPGQGQQIGQGQQGYYPTSPQHTGQRQQPVQGQQIGQGQQPEQGQQPGQWQQGYYPTSPQQLGQGQQPGQWQQSGQGQQGHYPTSLQQPGQGQQGHYLASQQQPAQGQQGHYPASQQQPGQGQQGHYPASQQQPGQGQQGHYPASQQEPGQGQQGQIPASQQQPGQGQQGHYPASLQQPGQQGHYPTSLQQLGQGQQIGQPGQKQQPGQGQQTGQGQQPEQEQQPGQGQQGYYPTSLQQPGQGQQQGQGQQGYYPTSLQQPGQGQQGHYPASLQQPGQGQGQPGQRQQPGQGQHPEQGQQPGQGQQGYYPTSPQQPGQGQQLGQGQQGYYPTSPQQPGQGQQPGQGQQGHCPMSPQQTGQAQQLGQGQQIGQVQQPGQGQQGYYPTSLQQPGQGQQSGQGQQSGQGHQPGQGQQSGQEKQGYDSPYHVSAEQQAASPMVAKAQQPATQLPTVCRMEGGDALSASQ

SEQ ID NO: 7 - protein, 48 aa, Triticum aestivum
HGKSQVLQQSTYQLLQELCCQHLWQIPEQSQCQAIHNVVHAIILHQQQ

SEQ ID NO: 8 - protein, 53 aa, Triticum aestivum
HGSSQVLQESTYQLVQQLCCQQLWQIPEQSRCQAIHNVVHAIILHQQHHHHQQ

SEQ ID NO: 9 - protein, 48 aa, Triticum aestivum
HASSQVLQQSSYQQLQQLCCQQLFQIPEQSRCQAIHNVVHAIILHHHQ

SEQ ID NO: 10 - protein, 49 aa, Triticum aestivum
VLQQSTYQLVQQFCCQQLWQIPEQSRCQAIHNVVHAIILHQQQQQQQQQ

SEQ ID NO: 11 - protein, 60 aa, Triticum aestivum
HASSQVLQQSTYQLLQQLCCQQLLQIPEQSQCQAIHNVAHAIIMHQQQQQQQEQKQQLQQ

SEQ ID NO: 12 - protein, 42 aa, Triticum aestivum
QVVDQQLAGRLPWSTGLQMRCCQQLRDVSAKCRSVAVSQVAR

SEQ ID NO: 13 - protein, 305 aa, Hordeum vulgare
MKILIILTILAMATTFATSEMQVNPSVQVQPTQQQPYPESQQPFISQSQQQFPQPQQPFPQQPQQPFPQSQQQCLQQPQHQFPQPTQQFPQRPLLPFTHPFLTFPDQLLPQPPHQSFPQPPQSYPQPPLQPFPQPPQQKYPEQPQQPFPWQQPTIQLYLQQQLNPCKEFLLQQCRPVSLLSYIWSKIVQQSSCRVMQQQCCLQLAQIPEQYKCTAIDSIVHAIFMQQGQRQGVQIVQQQPQPQQVGQCVLVQGQGVVQPQQLAQMEAIRTLVLQSVPSMCNFNVPPNCSTIKAPFVGVVTGVGGQ

SEQ ID NO: 14 - protein, 289 aa, Hordeum vulgare
ITTTTMQFNPSGLELERPQQLFPQWQPLPQQPPFLQQEPEQPYPQQQPLPQQQPFPQQPQLPHQHQFPQQLPQQQFPQQMPLQPQQQFPQQMPLQPQQQPQFPQQKPFGQYQQPLTQQPYPQQQPLAQQQPSIEEQHQLNLCKEFLLQQCTLDEKVPLLQSVISFLRPHISQQNSCQLKRQQCCQQLANINEQSRCPAIQTIVHAIVMQQQVQQQVGHGFVQSQLQQLGQGMPIQLQQQPGQAFVLPQQQAQFKVVGSLVIQTLPMLCNVHVPPYCSPFGSMATGSGGQ

SEQ ID NO: 15 - protein, 293 aa, Hordeum vulgare
MKTFLIFALLAIAATSTIAQQQPFPQQPIPQQPQPYPQQPQPYPQQPFPPQQPFPQQPVPQQPQPYPQQPFPPQQPFPQQPPFWQQKPFPQQPPFGLQQPILSQQQPCTPQQTPLPQGQLYQTLLQLQIQYVHPSILQQLNPCKVFLQQQCSPVPVPQRIARSQMLQQSSCHVLQQQCCQQLPQIPEQFRHEAIRAIVYSIFLQEQPQQLVEGVSQPQQQLWPQQVGQCSFQQPQPQQVGQQQQVPQSAFLQPHQIAQLEATTSIALRTLPMMCSVNVPLYRILRGVGPSVGV

SEQ ID NO: 16 - protein, 348 aa, Secale cereale
MKTLLMLAILAMATTIATANMQVNPSGQVQRPQQQPFPQPQQSSPQQPQQPFPQQPQQPVPQQSQQSFPQTQQPQQPLPQPQQPQQLFPQPQQSSPQQSQQVTSQPQQPFPQAQPPQQSCPQSQQPYPQEPQQLFPQSQQPQQPFPQPQQPQQPFPQPQPQTQQSIPQPQQPFPQYQEPFPQVHQPQQPSPQQQQPSIQLSLQQQLNPCKNVLLQQCSPVALVSSLRSKIFPQSECQVMQQQCCQQLAQIPQQLQCAAIHSVVHAIIMQQEQREGVQILLPQSHQQHVGQGALAQVQGIIQPQQLSQLEVVRSLVLQNLPTMCNIYVPRQCSTIQAPFASIVTGIVGH

SEQ ID NO: 17 - protein, 220 aa, Avena sativa
MKTFLIFALLAMAATMATAQFDPSEQYQPYPEQQQPILQQQQMLLQQQQQMLLQQQPLLQVLQQQLNPCRQFLVQQCSPVAVVPFLRSQILQQSSCQVMRQQCCRQLEQIPEQLRCPAIHSVVQAIIMQQQQFFQPQMQQQFFQPQMQQVTQGIFQPQMQQVTQGIFQPQLQQVTQGIFQPQMQGQIEGMRAFALQALPAMCDVYVPPHCPVATAPLGGF

SEQ ID NO: 18 - protein, 182 aa, Avena sativa
TTTVQYNPSEQYQPYPEQQEPFVQQQPFVQQQQQPFVQQQQMFLQPLLQQQLNPCKQFLVQQCSPVAVVPFLRSQILRQAICQVARQQCCRQLAQIPEQLRCPAIHSVVQAIILQQQQQQQFFQPQLQQQVFQPQLQQVFNQPQQQAQFEGMRAFALQALPAMCDVYVPPQCPVATAPLGGF

SEQ ID NO: 19 - protein, 55 aa, Triticum aestivum
LARSQMWQQSSCHVMQQQCCQQLQQIPEQSRYEAIRAIIYSIILQEQQQGFVQPQ

SEQ ID NO: 20 - protein, 57 aa, Triticum aestivum
SLWSIIWPQSDCQVMRQQCCQQLAQIPQQLQCAAIHSVVHSIIMQQQQQQQQQQGID

SEQ ID NO: 21 - protein, 57 aa, Triticum aestivum
LARSQMLQQSSCHVMQQQCCQQLPQIPQQSRYQAIRAIIYSIILQEQQQVQGSIQSQ

SEQ ID NO: 22 - protein, 49 aa, Hordeum vulgare
IARSQMLQQSSCHVLQQQCCQQLPQIPEQFRHEAIRAIVYSIFLQEQPQ

SEQ ID NO: 23 - protein, 49 aa, Hordeum vulgare
IARSQMLQQSSCHVLQQQCCQQLPQIPEQLRHEAVRAIVYSIVLQEQSL

SEQ ID NO: 24 - protein, 54 aa, Hordeum vulgare
YIWSKIVQQSSCRVMQQQCCLQLAQIPEQYKCTAIDSIVHAIFMQQGQRQGVQI

SEQ ID NO: 25 - protein, 50 aa, Hordeum vulgare
FLRPHISQQNSCQLKRQQCCQQLANINEQSRCPAIQTIVHAIVMQQQVQQ

SEQ ID NO: 26 - protein, 60 aa, Avena sativa
FLRSQILRQAICQVARQQCCRQLAQIPEQLRCPAIHSVVQAIILQQQQQQQFFQPQLQQQ

SEQ ID NO: 27 - protein, 57 aa, Avena sativa
FLRSQILQQSSCQVMRQQCCRQLEQIPEQLRCPAIHSVVQAIIMQQQQFFQPQMQQQ

SEQ ID NO: 28 - protein, 74 aa, Triticum aestivum
ILQQILQQQLIPCRDVVLQQHNIAHGSSQVLQQSTYQLLQQLCCQQLWQIPEQSRCQAIHNVVHAIILHQQQQQ

SEQ ID NO: 29 - protein, 19 aa, Triticum aestivum
LWQIPEQSRCQAIHNVVHA

SEQ ID NO: 30 - protein, 22 aa, Triticum aestivum
LAAQLWQIPEQSRAQAIHNVVH

SEQ ID NO: 31 - protein, 19 aa, Triticum aestivum
LWQIPEQSQCQAIHNVVHA

SEQ ID NO: 32 - protein, 19 aa, Triticum aestivum
LWQIPEQSQCQAIHNVVQA

SEQ ID NO: 33 - protein, 23 aa, Triticum aestivum
RCCQQLRDVSAKCRSVAVSQVAR

SEQ ID NO: 34 - protein, 23 aa, Triticum aestivum
RAAQQLRDVSAKARSVAVSQVAR

SEQ ID NO: 35 - protein, 36 aa, Triticum aestivum
VQQLCCQQLWQIPEQSRCQAIHNVVHAIILHQQQQQ

SEQ ID NO: 36 - protein, 35 aa, Triticum aestivum
LQELCCQHLWQIPEQSQCQAIHNVVHAIILHQQQK

SEQ ID NO: 37 - protein, 39 aa, Triticum aestivum
VQQLCCQQLWQIPEQSRCQAIHNVVHAIILHQQHHHHQQ

SEQ ID NO: 38 - protein, 35 aa, Triticum aestivum
LQQLCCQQLFQIPEQSRCQAIHNVVHAIILHHHQQ

SEQ ID NO: 39 - protein, 39 aa, Triticum aestivum
VQQFCCQQLWQIPEQSRCQAIHNVVHAIILHQQQQQQQQ

SEQ ID NO: 40 - protein, 40 aa, Triticum aestivum
LQQLCCQQLLQIPEQSQCQAIHNVAHAIIMHQQQQQQQEQ

SEQ ID NO: 41 - protein, 35 aa, Triticum aestivum
LQQLCCQQLWQIPEQSRCQAIHNVVHAIILHQQQR

SEQ ID NO: 42 - protein, 40 aa, Triticum aestivum
LQQLCCQQLLQIPEQSRCQAIHNVVHAIIMHQQEQQQQLQ

SEQ ID NO: 43 - protein, 36 aa, Triticum aestivum
LQELCCQHLWQIPEQSQCQAIHNVVHAIILHQQQQK

SEQ ID NO: 44 - protein, 31 aa, Triticum aestivum
LQQLCCQQLWQIPEQSRCQAIHNVVHAIILH

SEQ ID NO: 45 - protein, 271 aa, Zea mays
MRVLLVALALLALAASATSTHTSGGCGCQPPPPVHLPPPVHLPPPVHLPPPVHLPPPVHLPPPVHLPPPVHVPPPVHLPPPPCHYPTQPPRPQPHPQPHPCPCQQPHPSPCQLQGTCGVGSTPILGQCVEFLRHQCSPTPQPHPQPHPCPCQQPHPSPCQLQGTCGVGSTPILGQCVEFLRHQCSPTATPYCSPQCQSLRQQCCQQLRQVEPQHRYQAIFGLVLQSILQQQPQSGQVAGLLAAQIAQQLTAMCGLQQPTPCPYAAAGGVPH

SEQ ID NO: 46 - protein, 308 aa, Zea mays
MKLVLVVLAFIALVSSVSCTQTGGCSCGQQQSHEQQHHPQQHHPQKQQHQPPPQHHQQQQHQQQQVHMQPQKHQQQQEVHVQQQQQQPQHQQQQQQQQHQQQHQCEGQQQHHQQSQGHVQQHEQSHEQHQGQSHEQQHQQQFQGHDKQQQPQQPQQYQQGQEKSQQQQCHCQEQQQTTRCSYNYYSSSSNLKNCHEFLRQQCSPLVMPFLQSRLIQPSSCQVLQQQCCHDLRQIEPQYIHQAIYNMVQSIIQEEQQQQPCELCGSQQATQSAVAILTAAQYLPSMCGLYHSYYQNNPCSSNDISGVCN

SEQ ID NO: 47 - protein, 264 aa, Hordeum vulgare
QQPVSRQPQQIIPQQPQQPFPLQPQQPQPFPQQPIPQQPQPYPQQPQSFPQQPFPSQQPFPQQPPFWQQQPVLSQQQPCTQDQTPLLQEQQDQMLVQVQIPFVHPSILQQLNPCKVFLQQQCSPLAMSQRIARSQMLQQSSCHVLQQQCCQQLPQIPEQLRHEAVRAIVYSIVLQEQSLQLVQGVSQPQQQSQQQQVGQCSFQQPQPQQGQQQQVPQSVFLQPHQIAQLEATTSIALRTLPTMCSVNVPLYRIVPLAIDTRVGV

SEQ ID NO: 48 - protein, 293 aa, Hordeum vulgare; identical to SEQ ID NO: 15

SEQ ID NO: 49 - protein, 307 aa, Triticum aestivum
MKTFLILALLAIVATTARIAVRVPVPQLQPQNPSQQQPQEQVPLVQQQQFPGQQQPFPPQQPYPQPQPFPSQQPYLQLQPFPQPQLPYPQPQLPYPQPQLPYPQPQPFRPQQPYPQSQPQYSQPQQPISQQQQQQQQQQQQKQQQQQQQQILQQILQQQLIPCRDVVLQQHSIAYGSSQVLQQSTYQLVQQLCCQQLWQIPEQSRCQAIHNVVHAIILHQQQQQQQQQQQQPLSQVSFQQPQQQYPSGQGSFQPSQQNPQAQGSVQPQQLPQFEEIRNLALETLPAMCNVYIPPYCTIAPVGIFGTN

SEQ ID NO: 50 - protein, 289 aa, Hordeum vulgare; identical to SEQ ID NO: 14

SEQ ID NO: 51 - protein, 305 aa, Hordeum vulgare; identical to SEQ ID NO: 13

SEQ ID NO: 52 - protein, 5 aa, Artificial Sequence
  VARIANT 2: Xaa = Glu or Asp
  VARIANT 5: Xaa = Any Amino Acid
  Synthetic Construct
IXGRX

SEQ ID NO: 53 - protein, 6 aa, Artificial Sequence
  Synthetic Construct
LVPRGS

SEQ ID NO: 54 - protein, 6 aa, Artificial Sequence
  VARIANT 6: Xaa = Any Amino Acid
  Synthetic Construct
DDDDKX

SEQ ID NO: 55 - protein, 23 aa, Artificial Sequence
  VARIANT 1, 4, 12, 13: Xaa = any hydrophobic amino acid (optionally Leu, Ile, Val, or Ala)
  VARIANT 2, 8: Xaa = Any Amino Acid
  VARIANT 3, 11, 19, 23: Xaa = any hydrophilic amino acid (optionally Gln, Asn, Ser, His, Arg, and some cases Ala)
  VARIANT (5)..(7): Xaa = sequence containing two hydrophilic amino acids (optionally Pro, Gln, Asn, Ser) and an anionic amino acid (optionally Glu or Asp)
  VARIANT 9, 14, 18: Xaa = any cationic amino acid (optionally His, Arg, Lys), in some cases replaced by Gln, Ile, Leu, Tyr, Phe, Asp, or Glu
  VARIANT 10: Xaa = Cys, His, or Tyr
  VARIANT (15)..(17), (20)..(22): Xaa = sequence containing two hydrophobic amino acids (optionally Ala, Val, Leu, Ile, Phe or Met) and one hydrophilic amino acid (optionally Gly, Thr, Asn, Ser)
  Synthetic Construct
XXXXXXXXXXXXXXXXXXXXXXX

(19) United States
(12) Patent Application Publication
(10) Pub. No.: US 2024/0287533 A1
(43) Pub. Date: Aug. 29, 2024

(54) GLUTEN MODIFICATION

(71) Applicants: The Regents of the University of California, Oakland, CA (US); Beth Israel Deaconess Medical Center, Boston, MA (US)

(72) Inventors: Gerard C. L. WONG, Los Angeles, CA (US); Jaime DE ANDA, Los Angeles, CA (US); Wujing XIAN, Los Angeles, CA (US); Xinhua CHEN, Boston, MA (US); Ciaran KELLY, Boston, MA (US)

(73) Assignees: The Regents of the University of California, Oakland, CA (US); Beth Israel Deaconess Medical Center, Boston, MA (US)

(21) Appl. No.: 18/570,935

(22) PCT Filed: Jun. 29, 2022
(86) PCT No.: PCT/US2022/035575

§ 371 Date: Dec. 15, 2023

Related U.S. Application Data
(60) Provisional application No. 63/216,365, filed on Jun. 29, 2021.

(51) Int. Cl. C12N 15/82; C07K 14/415

(52) U.S. Cl. CPC C12N 15/8242; C07K 14/415

(57) ABSTRACT

The present disclosure provides methods for modifying gluten polypeptides to produce gluten polypeptides with a reduced inflammatory potential, as well as modified plants and compositions comprising modified gluten polypeptides with a reduced inflammatory potential.

### CROSS-REFERENCE TO RELATED APPLICATIONS

This application claims the benefit of U.S. Provisional Application No. 63/216,365, filed on Jun. 29, 2021, which is incorporated herein by reference in its entirety.

### STATEMENT REGARDING FEDERALLY-SPONSORED RESEARCH

This invention was made with government support under Grant Numbers 1650604 and 1808459, awarded by the National Science Foundation. The government has certain rights in the invention.

### SUBMISSION OF SEQUENCE LISTING ON ASCII TEXT FILE

The content of the following submission on ASCII text file is incorporated herein by reference in its entirety: a computer readable form (CRF) of the Sequence Listing (file name: 262232002340SEQLIST.TXT, date recorded: Jun. 29, 2022, size: 64,557 bytes).

### FIELD OF THE INVENTION

The present disclosure relates to methods for modifying gluten polypeptides to produce gluten polypeptides with reduced inflammatory potential, and to plants and compositions comprising such modified gluten polypeptides.

### BACKGROUND OF THE INVENTION

Gluten represents about 90% of the total protein composition in wheat flour, with gliadins (about 50%) and glutenins (about 50%) as building blocks (Biesiekierski, J Gastroenterology and Hepatology, 32:78-81 (2017)). When worked as a dough, gliadins and glutenins create an elastic network of interlinked disulfide bonds, giving rise to gluten.

Of the two components of gluten, the role of gliadins in gluten-related disorders, e.g., Celiac disease (CD), gluten allergy, and non-celiac gluten sensitivity, has been the subject of ample research (Luca et al., World J. Gastroenterol, 21(23):7110-7119 (2015); Hischenhuber et al., Aliment. Pharmacol. Ther, 23(5), 559-575 (2006); Mensiena et al., Mol. Nutr. Food Res, 62:1800716 (2018)). Extant results can be summarized as follows: the breakdown of gliadins leads to immunogenic fragments that provoke the immune system of patients. Intestinal and extra-intestinal symptoms associated with gluten-related disorders include abdominal pain, eczema and/or rash, headache, “foggy mind”, depression and diarrhea, among others (Igbinedion et al., World J. Gastroenterol, 23(40):7201-7210 (2017); Pietzak et al., J Parenteral and Enteral Nutrition, 36:68S-75S (2012)). The rapid and wide response of innate immunity could explain the broad set of intestinal and extra-intestinal symptoms associated with gluten-related disorders.

In CD patients, the chronic interstitial inflammatory reaction to gluten has been attributed to proline and glutamine-rich peptide fragments from gliadins. Specifically, a 33-mer peptide in α2-gliadin was demonstrated to be a potent inducer of T-cells from patients with CD (Hischenhuber et al., Aliment. Pharmacol. Ther, 23(5), 559-575 (2006); Shan et al., Science, 2275-2279 (2002); Sapone et al., BMC Med, 10, 13 (2012); Caio et al., BMC Med, 17(1):142 (2019)).

It is also known that the adaptive immune response observed in CD patients only accounts for a small fraction of all the gluten-related disorders. In the US, about 1% of the population is diagnosed with CD, whereas up to 6% of the population has been estimated to be afflicted with gluten allergy or gluten sensitivity (Luca et al., World J. Gastroenterol, 21(23):7110-7119 (2015); Igbinedion et al., World J. Gastroenterol, 23(40):7201-7210 (2017)).

Accordingly, there is a need in the art for developing methods for modifying gluten polypeptides to suppress innate immune responses, inflammation, and symptoms associated with gluten-related disorders.

All references cited herein, including patents, patent applications and publications, are hereby incorporated by reference in their entirety. To the extent that any reference incorporated by reference conflicts with the instant disclosure, the instant disclosure shall control.

### BRIEF SUMMARY OF THE INVENTION

In one aspect, provided herein is a method for producing a gluten polypeptide with a reduced inflammatory potential, the method comprising: (a) providing an amino acid sequence of a gluten polypeptide; (b) selecting an inflammatory amino acid sequence in gluten (ISG) in the amino acid sequence of the gluten polypeptide; (c) introducing one or more alterations in the ISG, wherein the one or more alterations are selected from: (i) a substitution of one or more amino acid residues, (ii) a deletion of one or more amino acid residues, (iii) an insertion of one or more amino acid residues, and any combination thereof, thereby generating a modified gluten polypeptide amino acid sequence; and (d) producing a gluten polypeptide comprising the modified gluten polypeptide amino acid sequence generated in step (c), thereby producing a gluten polypeptide with a reduced inflammatory potential.

In another aspect, provided herein is a method for producing a modified plant, plant tissue, plant organ, plant part, or plant cell that comprises a gluten polypeptide with a reduced inflammatory potential, the method comprising introducing a nucleic acid molecule encoding a gluten polypeptide with a reduced inflammatory potential into the plant, plant tissue, plant organ, plant part, or plant cell, wherein the gluten polypeptide with a reduced inflammatory potential comprises an amino acid sequence comprising one or more alterations in an ISG relative to a corresponding wild type gluten polypeptide, wherein the one or more alterations are selected from: (i) a substitution of one or more amino acid residues, (ii) a deletion of one or more amino acid residues, (iii) an insertion of one or more amino acid residues, and any combination thereof, thereby producing a modified plant, plant tissue, plant organ, plant part, or plant cell comprising a gluten polypeptide with a reduced inflammatory potential. In some embodiments, the method further comprises reducing the expression of one or more endogenous gluten polypeptides in the plant, plant tissue, plant organ, plant part, or plant cell. In some embodiments, the method further comprises introducing one or more alterations in one or more endogenous gluten polypeptides in the plant, plant tissue, plant organ, plant part, or plant cell. In some embodiments, the one or more alterations comprise a deletion of a gene encoding the one or more endogenous gluten polypeptides, or a part thereof, in the plant, plant tissue, plant organ, plant part, or plant cell. In some embodiments, the plant, plant tissue, plant organ, plant part, or plant cell is a wheat, a rye, a barley or an oat plant, plant tissue, plant organ, plant part, or plant cell.

In some embodiments of any of the methods provided herein, the gluten polypeptide is a gluten polypeptide from wheat, rye, barley or oat. In some embodiments, the gluten polypeptide is a gliadin polypeptide, a glutenin polypeptide, a hordein polypeptide, a secalin polypeptide, or an avenin polypeptide.

In some embodiments of any of the methods provided herein, the ISG is present in a non-repetitive region 1 (NRR1) of the gluten polypeptide. In some embodiments of any of the methods provided herein, the ISG comprises the amino acid sequence Hb-Vr-Hp-Hb-X-Vr-+-C-Hp-Hb-Hb-+-B-+-Hp-B-Hp (SEQ ID NO: 55), wherein: “Hb” is a hydrophobic amino acid, optionally wherein the hydrophobic amino acid is selected from the group consisting of leucine (L), isoleucine (I), valine (V), and alanine (A); “Hp” is a hydrophilic amino acid, optionally wherein the hydrophilic amino acid is selected from the group consisting of glutamine (Q), asparagine (N), serine (S), histidine (H), arginine (R), and A; “X” is a 3-mer sequence comprising two hydrophilic amino acids and an anionic amino acid, optionally wherein the hydrophilic amino acids are selected from the group consisting of proline (P), Q, N, and S, and the anionic amino acid is glutamate (E) or aspartate (D); “B” is a 3-mer sequence comprising hydrophobic amino acids and one hydrophilic amino acid, optionally wherein the hydrophobic amino acids are selected from the group consisting of A, V, L, I, phenylalanine (F), and methionine (M), and the hydrophilic amino acid is selected from the group consisting of glycine (G), threonine (T), N, and S; “C” is cysteine, H or tyrosine (Y); “+” is an amino acid selected from the group consisting of H, R, lysine (K), Q, I, L, Y, F, D, and E; and “Vr” is any amino acid. In some embodiments, the gluten polypeptide is a gliadin polypeptide, a glutenin polypeptide, or a gluten or gluten-related polypeptide from a cereal grain, optionally wherein the cereal grain is barley, oat, or corn. In some embodiments, the ISG comprises the amino acid sequence ILQQILQQQLIPCRDVVLQQHNIAHGSSQVLQQSTYQLLQQLCCQQLWQIPEQSRCQAIH NVVHAIILHQQQQQ (SEQ ID NO: 28). In some embodiments, the ISG comprises the amino acid sequence LWQIPEQSRCQAIHNVVHA (SEQ ID NO: 29). In some embodiments, the gluten polypeptide is a gliadin polypeptide, optionally wherein the gliadin polypeptide is an α-, β-, γ-, or ω-gliadin polypeptide. In some embodiments, the ISG comprises the amino acid sequence RCCQQLRDVSAKCRSVAVSQVAR (SEQ ID NO: 33). In some embodiments, the gluten polypeptide is a glutenin polypeptide, optionally wherein the glutenin polypeptide is a high molecular weight glutenin polypeptide. In some embodiments, the one or more alterations in the ISG comprise one or more of: an insertion of one or more negatively charged amino acids in the sequence of the ISG, optionally wherein the negatively charged amino acids are D or E; a substitution of one or more amino acid residues in the ISG sequence with a negatively charged amino acid, optionally wherein the negatively charged amino acid is D or E; a substitution of “+”, and/or “Hp” if “Hp” is a positively charged amino acid, with any amino acid residue, optionally wherein the positively charged amino acid residue is K, R, or H; a substitution of one or more positively charged amino acids at position “B” with any amino acid if “B” comprises any of K, R, or H; a deletion of “+”, and/or “Hp” if “Hp” is a positively charged amino acid, optionally wherein the positively charged amino acid K, R, or H; a deletion of one or more positively charged amino acids at position “B” if “B” comprises any of K, R, or H; an insertion of one or more amino acids; a deletion of one or more amino acids; a deletion of position “C”; a substitution of position “C” with any amino acid; a substitution of one or more bulky hydrophobic amino acids with any amino acid, optionally wherein the bulky hydrophobic amino acids are F, Y, or W; a substitution of one or more hydrophobic amino acids to a hydrophilic amino acid, optionally wherein the hydrophilic amino acid is selected from the group consisting of S, T, N, Q, G, P, C, K, R, H, K, D, and E, and the hydrophobic amino acids are selected from the group consisting of A, L, I, V, M, Y, W, or F; a substitution of one or more hydrophilic amino acids to a hydrophobic amino acid, optionally wherein the hydrophobic amino acid is selected from the group consisting of A, L, I, V, M, Y, W, and F, and the hydrophilic amino acids are selected from the group consisting of S, T, N, Q, G, P, C, K, R, H, K, D, and E; a substitution of a proline amino acid residue in “X” with any amino acid if “X” contains a proline; and/or a deletion of proline (P) in “X” with any amino acid, if “X” contains a proline. In some embodiments, the gluten polypeptide with a reduced inflammatory potential comprises a modified ISG comprising the sequence LAAQLWQIPEQSRAQAIHNVVH (SEQ ID NO: 30), LWQIPEQSQCQAIHNVVHA (SEQ ID NO: 31), or LWQIPEQSQCQAIHNVVQA (SEQ ID NO: 32). In some embodiments, the gluten polypeptide with a reduced inflammatory potential comprises a modified ISG comprising the sequence RAAQQLRDVSAKARSVAVSQVAR (SEQ ID NO: 34).

In some embodiments of any of the methods provided herein, the one or more alterations in an ISG comprise a substitution of one or more amino acid residues in the ISG sequence to the corresponding amino acid residue in a corresponding polypeptide from corn. In some embodiments of any of the methods provided herein, the one or more alterations in an ISG comprise a substitution of one or more amino acid residues in the ISG sequence to the corresponding amino acid residue in a corresponding gluten or gluten-related polypeptide from corn.

In some embodiments of any of the methods provided herein, the gluten polypeptide is a gliadin polypeptide. In some embodiments, the gliadin polypeptide is an α/β-gliadin polypeptide. In some embodiments, the α/β-gliadin polypeptide is an α/β-gliadin A-I polypeptide. In some embodiments, the α/β-gliadin A-I polypeptide comprises the amino acid sequence of SEQ ID NO: 1, or an amino acid sequence having at least about 85%, at least about 90%, at least about 95%, at least about 96%, at least about 97%, at least about 98%, at least about 99%, or 100% sequence identity to the amino acid sequence of SEQ ID NO: 1. In some embodiments, the ISG comprises the amino acid sequence of amino acid residues 159-206 of SEQ ID NO: 1, or of amino acid residues corresponding to amino acid residues 159-206 of an α/β-gliadin A-I polypeptide. In some embodiments, the ISG comprises the amino acid sequence of SEQ ID NO: 7, or an amino acid sequence having at least about 85%, at least about 90%, at least about 95%, at least about 96%, at least about 97%, at least about 98%, at least about 99%, or 100% sequence identity to the amino acid sequence of SEQ ID NO: 7. In some embodiments, the α/β-gliadin polypeptide is an α/β-gliadin A-II polypeptide. In some embodiments, the α/β-gliadin A-II polypeptide comprises the amino acid sequence of SEQ ID NO: 2, or an amino acid sequence having at least about 85%, at least about 90%, at least about 95%, at least about 96%, at least about 97%, at least about 98%, at least about 99%, or 100% sequence identity to the amino acid sequence of SEQ ID NO: 2. In some embodiments, the ISG comprises the amino acid sequence of amino acid residues 155-207 of SEQ ID NO: 2, or of amino acid residues corresponding to amino acid residues 155-207 of an α/β-gliadin A-II polypeptide. In some embodiments, the ISG comprises the amino acid sequence of SEQ ID NO: 8, or an amino acid sequence having at least about 85%, at least about 90%, at least about 95%, at least about 96%, at least about 97%, at least about 98%, at least about 99%, or 100% sequence identity to the amino acid sequence of SEQ ID NO: 8. In some embodiments, the α/β-gliadin polypeptide is an α/β-gliadin A-III polypeptide. In some embodiments, the α/β-gliadin A-III polypeptide comprises the amino acid sequence of SEQ ID NO: 3, or an amino acid sequence having at least about 85%, at least about 90%, at least about 95%, at least about 96%, at least about 97%, at least about 98%, at least about 99%, or 100% sequence identity to the amino acid sequence of SEQ ID NO: 3. In some embodiments, the ISG comprises the amino acid sequence of amino acid residues 152-199 of SEQ ID NO: 3, or of amino acid residues corresponding to amino acid residues 152-199 of an α/β-gliadin A-III polypeptide. In some embodiments, the ISG comprises the amino acid sequence of SEQ ID NO: 9, or an amino acid sequence having at least about 85%, at least about 90%, at least about 95%, at least about 96%, at least about 97%, at least about 98%, at least about 99%, or 100% sequence identity to the amino acid sequence of SEQ ID NO: 9. In some embodiments, the α/β-gliadin polypeptide is an α/β-gliadin A-IV polypeptide. In some embodiments, the α/β-gliadin A-IV polypeptide comprises the amino acid sequence of SEQ ID NO: 4, or an amino acid sequence having at least about 85%, at least about 90%, at least about 95%, at least about 96%, at least about 97%, at least about 98%, at least about 99%, or 100% sequence identity to the amino acid sequence of SEQ ID NO: 4. In some embodiments, the ISG comprises the amino acid sequence of amino acid residues 167-215 of SEQ ID NO: 4, or of amino acid residues corresponding to amino acid residues 167-215 of an α/β-gliadin A-IV polypeptide. In some embodiments, the ISG comprises the amino acid sequence of SEQ ID NO: 10, or an amino acid sequence having at least about 85%, at least about 90%, at least about 95%, at least about 96%, at least about 97%, at least about 98%, at least about 99%, or 100% sequence identity to the amino acid sequence of SEQ ID NO: 10. In some embodiments, the α/β-gliadin polypeptide is an α/β-gliadin A-V polypeptide. In some embodiments, the α/β-gliadin A-V polypeptide comprises the amino acid sequence of SEQ ID NO: 5, or an amino acid sequence having at least about 85%, at least about 90%, at least about 95%, at least about 96%, at least about 97%, at least about 98%, at least about 99%, or 100% sequence identity to the amino acid sequence of SEQ ID NO: 5. In some embodiments, the ISG comprises the amino acid sequence of amino acid residues 164-223 of SEQ ID NO: 5, or of amino acid residues corresponding to amino acid residues 164-223 of an α/β-gliadin A-V polypeptide. In some embodiments, the ISG comprises the amino acid sequence of SEQ ID NO: 11, or an amino acid sequence having at least about 85%, at least about 90%, at least about 95%, at least about 96%, at least about 97%, at least about 98%, at least about 99%, or 100% sequence identity to the amino acid sequence of SEQ ID NO: 11. In some embodiments, the gliadin polypeptide is a γ-gliadin B-I polypeptide. In some embodiments, the ISG comprises the amino acid sequence of SEQ ID NO: 19, or an amino acid sequence having at least about 85%, at least about 90%, at least about 95%, at least about 96%, at least about 97%, at least about 98%, at least about 99%, or 100% sequence identity to the amino acid sequence of SEQ ID NO: 19. In some embodiments, the gliadin polypeptide is a γ-gliadin B-II polypeptide. In some embodiments, the ISG comprises the amino acid sequence of SEQ ID NO: 20, or an amino acid sequence having at least about 85%, at least about 90%, at least about 95%, at least about 96%, at least about 97%, at least about 98%, at least about 99%, or 100% sequence identity to the amino acid sequence of SEQ ID NO: 20. In some embodiments, the gliadin polypeptide is a γ-gliadin B-III polypeptide. In some embodiments, the ISG comprises the amino acid sequence of SEQ ID NO: 21, or an amino acid sequence having at least about 85%, at least about 90%, at least about 95%, at least about 96%, at least about 97%, at least about 98%, at least about 99%, or 100% sequence identity to the amino acid sequence of SEQ ID NO: 21.

In some embodiments of any of the methods provided herein, the gluten polypeptide is a glutenin polypeptide. In some embodiments, the glutenin polypeptide is a high molecular weight subunit 12 glutenin polypeptide. In some embodiments, the glutenin polypeptide comprises the amino acid sequence of SEQ ID NO: 6, or an amino acid sequence having at least about 85%, at least about 90%, at least about 95%, at least about 96%, at least about 97%, at least about 98%, at least about 99%, or 100% sequence identity to the amino acid sequence of SEQ ID NO: 6. In some embodiments, the ISG comprises the amino acid sequence of amino acid residues 45-86 of SEQ ID NO: 6, or of amino acid residues corresponding to amino acid residues 45-86 of a high molecular weight subunit 12 glutenin polypeptide. In some embodiments, the ISG comprises the amino acid sequence of SEQ ID NO: 12, or an amino acid sequence having at least about 85%, at least about 90%, at least about 95%, at least about 96%, at least about 97%, at least about 98%, at least about 99%, or 100% sequence identity to the amino acid sequence of SEQ ID NO: 12.

In some embodiments of any of the methods provided herein, the gluten polypeptide is a hordein polypeptide. In some embodiments, the hordein polypeptide is a γ-hordein-1 polypeptide. In some embodiments, the γ-hordein-1 polypeptide comprises the amino acid sequence of SEQ ID NO: 13, or an amino acid sequence having at least about 85%, at least about 90%, at least about 95%, at least about 96%, at least about 97%, at least about 98%, at least about 99%, or 100% sequence identity to the amino acid sequence of SEQ ID NO: 13. In some embodiments, the ISG comprises the amino acid sequence of amino acid residues 182-235 of SEQ ID NO: 13, or of amino acid residues corresponding to amino acid residues 182-235 of a γ-hordein-1 polypeptide. In some embodiments, the ISG comprises the amino acid sequence of SEQ ID NO: 24, or an amino acid sequence having at least about 85%, at least about 90%, at least about 95%, at least about 96%, at least about 97%, at least about 98%, at least about 99%, or 100% sequence identity to the amino acid sequence of SEQ ID NO: 24. In some embodiments, the hordein polypeptide is a γ-hordein-3 polypeptide. In some embodiments, the γ-hordein-3 polypeptide comprises the amino acid sequence of SEQ ID NO: 14, or an amino acid sequence having at least about 85%, at least about 90%, at least about 95%, at least about 96%, at least about 97%, at least about 98%, at least about 99%, or 100% sequence identity to the amino acid sequence of SEQ ID NO: 14. In some embodiments, the ISG comprises the amino acid sequence of amino acid residues 165-214 of SEQ ID NO: 14, or of amino acid residues corresponding to amino acid residues 165-214 of a γ-hordein-3 polypeptide. In some embodiments, the ISG comprises the amino acid sequence of SEQ ID NO: 25, or an amino acid sequence having at least about 85%, at least about 90%, at least about 95%, at least about 96%, at least about 97%, at least about 98%, at least about 99%, or 100% sequence identity to the amino acid sequence of SEQ ID NO: 25. In some embodiments, the hordein polypeptide is a B1-hordein polypeptide. In some embodiments, the B1-hordein polypeptide comprises the amino acid sequence of SEQ ID NO: 15, or an amino acid sequence having at least about 85%, at least about 90%, at least about 95%, at least about 96%, at least about 97%, at least about 98%, at least about 99%, or 100% sequence identity to the amino acid sequence of SEQ ID NO: 15. In some embodiments, the ISG comprises the amino acid sequence of amino acid residues 160-208 of SEQ ID NO: 15, or of amino acid residues corresponding to amino acid residues 160-208 of a B1-hordein polypeptide. In some embodiments, the ISG comprises the amino acid sequence of SEQ ID NO: 22, or an amino acid sequence having at least about 85%, at least about 90%, at least about 95%, at least about 96%, at least about 97%, at least about 98%, at least about 99%, or 100% sequence identity to the amino acid sequence of SEQ ID NO: 22. In some embodiments, the hordein polypeptide is a B3-hordein polypeptide. In some embodiments, the ISG comprises the amino acid sequence of SEQ ID NO: 23, or an amino acid sequence having at least about 85%, at least about 90%, at least about 95%, at least about 96%, at least about 97%, at least about 98%, at least about 99%, or 100% sequence identity to the amino acid sequence of SEQ ID NO: 23.

In some embodiments of any of the methods provided herein, the gluten polypeptide is a secalin polypeptide. In some embodiments, the secalin polypeptide is a 75 k gamma secalin polypeptide. In some embodiments, the 75 k gamma secalin polypeptide comprises the amino acid sequence of SEQ ID NO: 16, or an amino acid sequence having at least about 85%, at least about 90%, at least about 95%, at least about 96%, at least about 97%, at least about 98%, at least about 99%, or 100% sequence identity to the amino acid sequence of SEQ ID NO: 16.

In some embodiments of any of the methods provided herein, the gluten polypeptide is an avenin polypeptide. In some embodiments, the avenin polypeptide is an avenin-3 polypeptide. In some embodiments, the avenin-3 polypeptide comprises the amino acid sequence of SEQ ID NO: 17, or an amino acid sequence having at least about 85%, at least about 90%, at least about 95%, at least about 96%, at least about 97%, at least about 98%, at least about 99%, or 100% sequence identity to the amino acid sequence of SEQ ID NO: 17. In some embodiments, the ISG comprises the amino acid sequence of amino acid residues 85-141 of SEQ ID NO: 17, or of amino acid residues corresponding to amino acid residues 85-141 of an avenin-3 polypeptide. In some embodiments, the ISG comprises the amino acid sequence of SEQ ID NO: 27, or an amino acid sequence having at least about 85%, at least about 90%, at least about 95%, at least about 96%, at least about 97%, at least about 98%, at least about 99%, or 100% sequence identity to the amino acid sequence of SEQ ID NO: 27. In some embodiments, the avenin polypeptide is an avenin-E polypeptide. In some embodiments, the avenin-E polypeptide comprises the amino acid sequence of SEQ ID NO: 18, or an amino acid sequence having at least about 85%, at least about 90%, at least about 95%, at least about 96%, at least about 97%, at least about 98%, at least about 99%, or 100% sequence identity to the amino acid sequence of SEQ ID NO: 18. In some embodiments, the ISG comprises the amino acid sequence of amino acid residues 71-130 of SEQ ID NO: 18, or of amino acid residues corresponding to amino acid residues 71-130 of an avenin-E polypeptide. In some embodiments, the ISG comprises the amino acid sequence of SEQ ID NO: 26, or an amino acid sequence having at least about 85%, at least about 90%, at least about 95%, at least about 96%, at least about 97%, at least about 98%, at least about 99%, or 100% sequence identity to the amino acid sequence of SEQ ID NO: 26.

In some embodiments of any of the methods provided herein, the gluten polypeptide with a reduced inflammatory potential comprises: (a) a reduced ability to remodel a cell membrane; (b) a reduced ability to access an endosomal compartment within a cell; (c) a reduced ability to mediate organization of innate immune ligands into ordered nanocrystalline structures; (d) a reduced ability to promote activation of Toll-Like Receptors (TLRs); and/or (e) a reduced ability to self-assemble into amyloidal or protofibril structures to activate formyl peptide receptor-like 1 (FPRL1) and/or formyl peptide receptor 2 (FPR2), as compared to a gluten polypeptide without the one or more alterations in the ISG. In some embodiments, the reduced ability to remodel a cell membrane comprises a reduced ability to permeabilize a cell membrane, as compared to a gluten polypeptide without the one or more alterations in the ISG. In some embodiments, the innate immune ligands comprise one or more nucleic acid molecules. In some embodiments, the one or more nucleic acid molecules comprise one or more double-stranded DNA (dsDNA), double-stranded RNA (dsRNA), single-stranded DNA (ssDNA), and/or single-stranded RNA (ssRNA) molecules.

In another aspect, provided herein is an isolated nucleic acid molecule comprising a nucleotide sequence encoding a gluten polypeptide with a reduced inflammatory potential produced by any of the methods provided herein.

In another aspect, provided herein is a vector comprising any nucleic acid molecule provided herein.

In another aspect, provided herein are host cells comprising any vector provided herein.

In another aspect, provided herein is a plant, plant tissue, plant organ, plant part, or plant cell comprising any nucleic acid or vector provided herein.

In another aspect, provided herein is a plant, plant tissue, plant organ, plant part, or plant cell comprising a gluten polypeptide with a reduced inflammatory potential produced by any of the methods provided herein.

In another aspect, provided herein is a modified plant, plant tissue, plant organ, plant part, or plant cell produced by any of the methods provided herein. In some embodiments, the plant, plant tissue, plant organ, plant part, or plant cell is a wheat, rye, barley or oat plant, plant tissue, plant organ, plant part, or plant cell.

In another aspect, provided herein is an isolated gluten polypeptide with a reduced inflammatory potential produced by any of the methods provided herein.

In another aspect, provided herein is an isolated gluten polypeptide with a reduced inflammatory potential obtained from a plant, plant tissue, plant organ, plant part, or plant cell comprising a gluten polypeptide with a reduced inflammatory potential produced by any of the methods provided herein.

In another aspect, provided herein is a composition comprising any gluten polypeptide with a reduced inflammatory potential provided herein. In some embodiments, the composition is an orally consumable composition, a foodstuff composition, a beverage product, a nutraceutical composition, a dietary supplement, an edible gel, a pharmaceutical composition, or a cosmetic or skin composition.

In another aspect, provided herein is a composition comprising any plant, plant tissue, plant organ, plant part, or plant cell provided herein. In some embodiments, the composition is an orally consumable composition, a foodstuff composition, a beverage product, a nutraceutical composition, a dietary supplement, an edible gel, a pharmaceutical composition, or a cosmetic or skin composition.

### BRIEF DESCRIPTION OF THE DRAWINGS

The patent or application file contains at least one drawing executed in color. Copies of this patent or patent application publication with color drawings will be provided by the office upon request and payment of the necessary fee.

FIG. 1 shows the identification of candidate immunomodulatory regions in α-gliadin isoforms using machine learning scans on nine isoforms from UniProtKB/Swiss-Prot. (Top panel) Probability scores indicate the probability of forming organized complexes with pathogen associated molecular patterns (PAMPs) to activate inflammation by the innate immune system. The color gradient indicates the averaged probability at each amino acid position as calculated from a window-scan scoring over the full sequence-window size of 19 amino acids. The white spaces with dotted lines represent missing amino acids at the aligned position for a given sequence. (Bottom panel) shows the conservation across each gene of the high scoring C-terminal tail of the NRR1. Colored amino acids are conserved across all nine genes presented in this figure. The consensus sequence is listed at the bottom.

FIG. 2 shows a sequence alignment of hordeins from barley (γ-hordein 1 and 3, B1- and B3-hordein), avenins from oats (avenin-3 and -E), and gluten-related proteins from corn (glutelin-2 and γ-zein 50 k). The highlighted region represents the vicinity of the non-repetitive region 1 (NRR1) in α-gliadin, while the bolded sequence is the identified 19-mer peptide, gld-1. The Glutelin-2 sequence corresponds to SEQ ID NO: 45; the 50-kDa-gamma-zein sequence corresponds to SEQ ID NO: 46; the B3-hordein sequence corresponds to SEQ ID NO: 47; the B1-hordein sequence corresponds to SEQ ID NO: 48; the Alpha-gliadin sequence corresponds to SEQ ID NO: 49; the Gamma-hordein-3 sequence corresponds to SEQ ID NO: 50; the Gamma-hordein-1 sequence corresponds to SEQ ID NO: 51; the Avenin-E sequence corresponds to SEQ ID NO: 18; and the Avenin-3 sequence corresponds to SEQ ID NO: 17.

FIG. 3 shows IL-8 production by THP-1 monocytes that were treated with medium as a control, double stranded DNA (dsDNA), the indicated gluten peptides (see, Table 2 and Examples 1-2, herein), or dsDNA+gluten peptide for 18 hours, as measured by enzyme-linked immunosorbent assay (ELISA). *denotes p<0.05, **denotes p<0.01, ***denotes p<0.001.

FIG. 4 shows IL-8 production by HT-29 cells that were treated with medium as a control, dsDNA, the indicated gluten peptides (see, Table 2 and Examples 1-2, herein), or dsDNA+gluten peptide for 18 hours, as measured by ELISA. *denotes p<0.05, **denotes p<0.01, ***denotes p<0.001.

FIG. 5A shows NF-κB/IL-8-induced luciferase activity in HEK293 cells transfected with pcDNA3.1 vector and treated with medium as a control, dsDNA, gld-1, or dsDNA+gld-1 for 4 hours. *denotes p<0.05, **denotes p<0.01, ***denotes p<0.001.

FIG. 5B shows NF-κB/IL-8-induced luciferase activity in HEK293 cells transfected with TLR9 plasmid and treated with medium as a control, dsDNA, gld-1, or dsDNA+gld-1 for 4 hours. *denotes p<0.05, **denotes p<0.01, ***denotes p<0.001. TLR9, Toll-like Receptor 9.

FIG. 6A shows gene expression (assessed by qPCR) of keratinocyte-derived chemokine (KC) in wild-type macrophages treated with medium as a control, dsDNA, gld-1, or dsDNA+gld-1 for 20 hours. *denotes p<0.05, **denotes p<0.01, ***denotes p<0.001.

FIG. 6B shows gene expression (assessed by qPCR) of IL-6 in wild-type macrophages treated with medium as a control, dsDNA, gld-1, or dsDNA+gld-1 for 20 hours. *denotes p<0.05, **notes d p<0.01, ***notes d p<0.001.

FIG. 6C shows gene expression (assessed by qPCR) of IL-6 in TLR9 knockout macrophages treated with medium as a control, dsDNA, gld-1, or dsDNA+gld-1 for 20 hours. *denotes p<0.05, **denotes p<0.01, ***denotes p<0.001.

FIG. 7 shows IL-8 production by THP-1 monocytes that were treated with medium as a control, dsDNA, gld-4, gld-4A, dsDNA+gld-4, or dsDNA+gld-4A for 18 hours, as measured by ELISA. *denotes p<0.05, **denotes p<0.01, ***denotes p<0.001.

FIG. 8A shows SAXS diffraction profiles of gld-1 and mutants, gld-2 and gld-3, complexed with dsDNA. Only gld-1 was able to organize dsDNA into a nano-crystalline lattice structure, as demonstrated by the two broad Bragg peaks as labeled in background subtracted curve (top right-inset of FIG. 8A). The peaks correspond to a rhombic columnar lattice with inter dsDNA spacing, d=4.0 nm, and tilt angle, γ=104.6°. To facilitate visualization, spectra were manually offset vertically by a multiplicative factor.

FIG. 8B shows a diagram of a dsDNA rhombic lattice presented to TLR9 receptors.

FIG. 8C shows a picture of macroscopic peptide-dsDNA complexes used for the SAXS exposures in FIG. 8A. All peptides were able to associate with dsDNA, but only gld-1 was able to form a well-structured complex.

FIG. 9 shows a schematic of the basic amino acid formula (SEQ ID NO: 55) of a complex forming gluten peptide CFGP as described in Example 2, herein. Hb is any hydrophobic amino acid [primarily leucine (L), isoleucine (I), valine (V), or alanine (A)]; Hp is any hydrophilic amino acid [primarily glutamine (Q), asparagine (N), serine (S), histidine (H), arginine (R), and in some cases A]; X is a 3-mer sequence containing two hydrophilic amino acids [primarily proline (P), Q, N, S] and an anionic amino acid [glutamate (E) or aspartate (D)]; B is a 3-mer sequence containing hydrophobic amino acids [primarily A, V, L, I, phenylalanine (F) or methionine (M)] and one hydrophilic amino acid [primarily glycine (G), threonine (T), N, S]; C stands for the amino acid cysteine [in some cases replaced by H or tyrosine (Y)]; the symbol + represents primary locations of cationic amino acids, i.e., H, R, and lysine (K) [in some cases replaced by Q, I, L, Y, F, D, or E]; and Vr can be any amino acid.

### DETAILED DESCRIPTION OF THE INVENTION

The present disclosure is based, at least in part, on the discovery of a class of inflammatory amino acid sequences that are present in polypeptides of the gluten family, and that cause inflammation, such as inflammation in the gut. Accordingly, provided herein are methods for modifying gluten polypeptides, e.g., to produce modified gluten polypeptides with reduced inflammatory potential. Also provided herein are modified plants and compositions comprising such modified gluten polypeptides.

The following description is presented to enable a person of ordinary skill in the art to make and use the various embodiments. Descriptions of specific devices, techniques, and applications are provided only as examples. Various modifications to the examples described herein will be readily apparent to those of ordinary skill in the art, and the general principles defined herein may be applied to other examples and applications without departing from the spirit and scope of the various embodiments. Thus, the various embodiments are not intended to be limited to the examples described herein and shown, but are to be accorded the scope consistent with the claims.

### Definitions

The use of the terms “a,” “an,” and “the,” and similar referents in the context of describing the disclosure (especially in the context of the following claims) are to be construed to cover both the singular and the plural, unless otherwise indicated herein or clearly contradicted by context.

The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. For example, if the range 10-15 is disclosed, then 11, 12, 13, and 14 are also disclosed.

Reference to “about” a value or parameter herein refers to the usual error range for the respective value readily known to the skilled person in this technical field. Reference to “about” a value or parameter herein includes (and describes) aspects that are directed to that value or parameter per se. For example, description referring to “about X” includes description of “X.”

As used herein, “gluten polypeptide” refers to any polypeptide or a fragment thereof that is a component of gluten, such as a gliadin polypeptide or a fragment thereof or a glutenin polypeptide or a fragment thereof. The term “gluten polypeptide” also encompasses a gluten-related polypeptide or a fragment thereof, such as a hordein polypeptide or a fragment thereof, a secalin polypeptide or a fragment thereof, or an avenin polypeptide or a fragment thereof, unless otherwise indicated herein or clearly contradicted by context.

The term “identity” with respect to polynucleotide and polypeptide sequences refers to sequence similarity between two polynucleotide sequences or between two polypeptide sequences. As used herein, percent (%) amino acid sequence identity with respect to two polypeptide sequences refers to the percentage of amino acid residues in the two sequences that are identical, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways that are within the skill in the art, for instance, using publicly available computer software such as BLAST, BLAST-2, ALIGN or MEGALIGN (DNASTAR) software. Those skilled in the art can determine appropriate parameters for measuring alignment, including any algorithms known in the art needed to achieve maximal alignment over the full-length of the sequences being compared.

As used herein, an “individual” refers to a mammal, such as a primate (e.g., a human or a non-human primate), or another mammal, e.g., a farm animal (e.g., a pig, cow, horse, sheep, goat, and the like), a domestic animal (e.g., a dog, cat or rodent), or a laboratory animal (e.g., rodents such as mice, rats, guinea pigs, and the like). In some cases, the individual is a human. The individual may be an individual of any age or sex. In some cases, an individual may have been previously diagnosed with or identified as having a gluten-related allergy, sensitivity, toxicity, disease or disorder. In some cases, an individual has not been previously diagnosed with or identified as having a gluten-related allergy, sensitivity, toxicity, disease or disorder.

As used herein, “inflammatory potential” refers to the ability of a gluten polypeptide to elicit inflammation in an individual, e.g., a human individual, upon ingestion of the gluten polypeptide by the individual. The phrase “inflammatory potential” also refers to the ability of a gluten polypeptide to elicit one or more symptoms of a gluten-related allergy, sensitivity, toxicity, disease or disorder upon ingestion of the gluten polypeptide by the individual. “Ingestion,” as used herein, encompasses oral ingestion (e.g., by eating or drinking) of a gluten polypeptide, as well as introduction of a gluten polypeptide into an individual by other means, such as by parenteral (e.g., subcutaneous, intramuscular, intradermal, or intravenous), inhalation (e.g., in solid or liquid forms), buccal (e.g., sub-lingual), transdermal, rectal, or topical routes. The phrase “reduced inflammatory potential” refers to a gluten polypeptide having a reduced “inflammatory potential” as compared to a reference gluten polypeptide. For example, a modified gluten polypeptide having a reduced inflammatory potential has a reduced inflammatory potential as compared to a reference unmodified gluten polypeptide.

It is to be understood that aspects and embodiments of the present disclosure described herein include “comprising,” “consisting,” and “consisting essentially of” aspects and embodiments.

The use of any and all examples, or exemplary language (e.g., “such as,” “exemplary” or “for example”) provided herein, is intended merely to better illuminate the embodiments of the disclosure and does not pose a limitation on the scope of the disclosure unless otherwise claimed. No language in the specification should be construed as indicating any non-claimed element as essential to the practice of the embodiments of the disclosure. All methods described herein can be performed in any suitable order unless otherwise indicated herein or otherwise clearly contradicted by context.

It is to be understood that one, some, or all of the properties of the various embodiments described herein may be combined to form other embodiments of the present disclosure. These and other aspects of the present disclosure will become apparent to one of skill in the art. These and other embodiments of the present disclosure are further described by the detailed description that follows.

All references cited herein are hereby incorporated by reference in their entirety.

### Gluten Polypeptides

In some aspects, the present disclosure relates to gluten and gluten-related polypeptides, and to methods of modifying such polypeptides to produce modified gluten and gluten-related polypeptides with a reduced inflammatory potential.

Gluten is a complex of two polypeptide fractions that have been classified based on their solubility in aqueous alcohols: the soluble gliadins and the insoluble glutenins. Gluten polypeptides, e.g., gliadins and glutenins, and gluten-related polypeptides, e.g., hordeins, secalins and avenins, are the major storage polypeptides of wheat and other plants, such as rye, barley, triticale and oat. Several hundred genes encoding gluten and gluten-related polypeptides have been described.

Both glutenin and gliadin polypeptides are characterized by a high glutamine and proline content. Gluten-related polypeptides, such as hordeins, secalins or avenins, are also generally characterized by a high proline and glutamine content. The high proline content of gluten and gluten-related polypeptides renders these polypeptides resistant to complete proteolytic digestion by gastric, pancreatic, and brush border enzymes in the human intestine, at least in part because those enzymes are deficient in prolyl endopeptidase activity. This can result in the accumulation of relatively large peptide fragments (e.g., as many as 50 amino acids in length) with a high proline and glutamine content in the small intestine.

### Gliadins

Gliadins are monomeric polypeptides that can have molecular weights (MWs) of around 28,000-55,000 Daltons, and isoelectric points of about pH 3.0-4.0. Several classes of gliadin polypeptides exist, including α-gliadin, β-gliadin, α/β-gliadin, γ-gliadin and ω-gliadin. Furthermore, each class of gliadin polypeptide includes numerous specific gliadin polypeptides. For example, the α/β-gliadin class of gliadin polypeptides in wheat (Triticum aestivum) includes, without limitation, α/β-gliadin A-I, α/β-gliadin A-II, α/β-gliadin A-III, α/β-gliadin A-IV, and α/β-gliadin A-V. One of ordinary skill in the art could readily determine the amino acid sequence of any gliadin polypeptide, e.g., from any class of gliadin polypeptides, for example using an NCBI database or other databases of polypeptide and gene sequences (e.g., UniProt or Genbank).

An exemplary amino acid sequence of an α/β-gliadin A-I polypeptide from wheat is available as UniProt Entry P04721 (e.g., available at the website: www[dot]uniprot[dot]org/uniprot/P04721), provided herein in SEQ ID NO: 1.

(SEQ ID NO: 1)
MKTFLILALLAIVATTATTAVRVPVPQLQPQNPSQQQPQEQVPLVQQQQ
FLGQQQPFPPQQPYPQPQPFPSQQPYLQLQPFLQPQLPYSQPQPFRPQQ
PYPQPQPQYSQPQQPISQQQQQQQQQQQQQQQQQQQIIQQILQQQLIPC
MDVVLQQHNIVHGKSQVLQQSTYQLLQELCCQHLWQIPEQSQCQAIHNV
VHAIILHQQQKQQQQPSSQVSFQQPLQQYPLGQGSFRPSQQNPQAQGSV
QPQQLPQFEEIRNLARK

An exemplary amino acid sequence of an α/β-gliadin A-II polypeptide from wheat is available as UniProt Entry P04722 (e.g., available at the website: www[dot]uniprot[dot]org/uniprot/P04722), and provided herein in SEQ ID NO: 2.

(SEQ ID NO: 2)
MKTFPILALLAIVATTATTAVRVPVPQLQLQNPSQQQPQEQVPLVQEQQ
FQGQQQPFPPQQPYPQPQPFPSQQPYLQLQPFPQPQLPYPQPQPFRPQQ
PYPQPQPQYSQPQQPISQQQQQQQQQQQQQQQILQQILQQQLIPCRDVV
LQQHNIAHGSSQVLQESTYQLVQQLCCQQLWQIPEQSRCQAIHNVVHAI
ILHQQHHHHQQQQQQQQQQPLSQVSFQQPQQQYPSGQGFFQPSQQNPQA
QGSFQPQQLPQFEEIRNLALQTLPAMCNVYIPPYCTIAPFGIFGTN

An exemplary amino acid sequence of an α/β-gliadin A-III polypeptide from wheat is available as UniProt Entry P04723 (e.g., available at the website: www[dot]uniprot[dot]org/uniprot/P04723), provided herein in SEQ ID NO: 3.

(SEQ ID NO: 3)
MKTFLILALLAIVATTATSAVRVPVPQLQPQNPSQQQPQEQVPLMQQQQ
QFPGQQEQFPPQQPYPHQQPFPSQQPYPQPQPFPPQLPYPQTQPFPPQQ
PYPQPQPQYPQPQQPISQQQAQQQQQQQQTLQQILQQQLIPCRDVVLQQ
HNIAHASSQVLQQSSYQQLQQLCCQQLFQIPEQSRCQAIHNVVHAIILH
HHQQQQQQPSSQVSYQQPQEQYPSGQVSFQSSQQNPQAQGSVQPQQLPQ
FQEIRNLALQTLPAMCNVYIPPYCSTTIAPFGIFGTN

An exemplary amino acid sequence of an α/β-gliadin A-IV polypeptide from wheat is available as UniProt Entry P04724 (e.g., available at the website: www[dot]uniprot[dot]org/uniprot/P04724), provided herein in SEQ ID NO: 4.

(SEQ ID NO: 4)
MKTFLILALRAIVATTATIAVRVPVPQLQPQNPSQQQPQKQVPLVQQQQ
FPGQQQPFPPQQPYPQQQPFPSQQPYMQLQPFPQPQLPYPQPQLPYPQP
QPFRPQQSYPQPQPQYSQPQQPISQQQQQQQQQQQQQQQILQQILQQQL
IPCRDVVLQQHSIAHGSSQVLQQSTYQLVQQFCCQQLWQIPEQSRCQAI
HNVVHAIILHQQQQQQQQQQQQQQQPLSQVCFQQSQQQYPSGQGSFQPS
QQNPQAQGSVQPQQLPQFEEIRNLALETLPAMCNVYIPPYCTIAPVGIF
GTN

An exemplary amino acid sequence of an α/β-gliadin A-V polypeptide from wheat is available as UniProt Entry P04725 (e.g., available at the website: www[dot]uniprot[dot]org/uniprot/P04725), provided herein in SEQ ID NO: 5.

(SEQ ID NO: 5)
MKTFLILALLAIVATTATTAVRVPVPQLQPQNPSQQQPQEQVPLVQQQQ
FPGQQQQFPPQQPYPQPQPFPSQQPYLQLQPFPQPQPFPPQLPYPQPQS
FPPQQPYPQQQPQYLQPQQPISQQQAQQQQQQQQQQQQQQQILQQILQQ
QLIPCRDVVLQQHNIAHASSQVLQQSTYQLLQQLCCQQLLQIPEQSQCQ
AIHNVAHAIIMHQQQQQQQEQKQQLQQQQQQQQQLQQQQQQQQQQPSSQ
VSFQQPQQQYPSSQVSFQPSQLNPQAQGSVQPQQLPQFAEIRNLALQTL
PAMCNVYIPPHCSTTIAPFGISGTN

### Glutenins

Glutenins are larger, multimeric polypeptides that can be more than 10,000,000 Daltons, with an average MW of about 3,000,000 Daltons. Isoelectric points for glutenins range from about pH 6.5-7.0. After reduction of disulphide bonds, the resulting glutenin subunits show a solubility in aqueous alcohols similar to gliadins. Based on primary structure, glutenin subunits have been divided into the high-molecular-weight (HMW) subunits (MW=67,000-88,000 Daltons), and the low-molecular-weight (LMW) subunits (MW=32,000-35,000 Daltons). One of ordinary skill in the art could readily determine the amino acid sequence of any glutenin polypeptide, e.g., from any class of glutenin polypeptides, for example using an NCBI database or other databases of polypeptide and gene sequences (e.g., UniProt or Genbank).

An exemplary amino acid sequence of a high molecular weight subunit 12 glutenin polypeptide from wheat is available as UniProt Entry P08488 (e.g., available at the website: www[dot]uniprot[dot]org/uniprot/P08488), provided herein in SEQ ID NO: 6.

(SEQ ID NO: 6)
MAKRLVLFAAVVIALVALTTAEGEASRQLQCERELQESSLEACRQVVDQ
QLAGRLPWSTGLQMRCCQQLRDVSAKCRSVAVSQVARQYEQTVVPPKGG
SFYPGETTPLQQLQQGIFWGTSSQTVQGYYPSVTSPRQGSYYPGQASPQ
QPGQGQQPGKWQEPGQGQQWYYPTSLQQPGQGQQIGKGKQGYYPTSLQQ
PGQGQQIGQGQQGYYPTSPQHTGQRQQPVQGQQIGQGQQPEQGQQPGQW
QQGYYPTSPQQLGQGQQPGQWQQSGQGQQGHYPTSLQQPGQGQQGHYLA
SQQQPAQGQQGHYPASQQQPGQGQQGHYPASQQQPGQGQQGHYPASQQE
PGQGQQGQIPASQQQPGQGQQGHYPASLQQPGQQGHYPTSLQQLGQGQQ
IGQPGQKQQPGQGQQTGQGQQPEQEQQPGQGQQGYYPTSLQQPGQGQQQ
GQGQQGYYPTSLQQPGQGQQGHYPASLQQPGQGQGQPGQRQQPGQGQHP
EQGQQPGQGQQGYYPTSPQQPGQGQQLGQGQQGYYPTSPQQPGQGQQPG
QGQQGHCPMSPQQTGQAQQLGQGQQIGQVQQPGQGQQGYYPTSLQQPGQ
GQQSGQGQQSGQGHQPGQGQQSGQEKQGYDSPYHVSAEQQAASPMVAKA
QQPATQLPTVCRMEGGDALSASQ

### Gluten-Related Polypeptides

An exemplary amino acid sequence of a γ-hordein-1 polypeptide from barley is available as UniProt Entry P17990 (e.g., available at the website: www[dot]uniprot[dot]org/uniprot/P17990), provided herein in SEQ ID NO: 13.

(SEQ ID NO: 13)
MKILIILTILAMATTFATSEMQVNPSVQVQPTQQQPYPESQQPFISQSQ
QQFPQPQQPFPQQPQQPFPQSQQQCLQQPQHQFPQPTQQFPQRPLLPFT
HPFLTFPDQLLPQPPHQSFPQPPQSYPQPPLQPFPQPPQQKYPEQPQQP
FPWQQPTIQLYLQQQLNPCKEFLLQQCRPVSLLSYIWSKIVQQSSCRVM
QQQCCLQLAQIPEQYKCTAIDSIVHAIFMQQGQRQGVQIVQQQPQPQQV
GQCVLVQGQGVVQPQQLAQMEAIRTLVLQSVPSMCNFNVPPNCSTIKAP
FVGVVTGVGGQ

An exemplary amino acid sequence of a γ-hordein-3 polypeptide from barley is available as UniProt Entry P80198 (e.g., available at the website: www[dot]uniprot[dot]org/uniprot/P80198), provided herein in SEQ ID NO: 14.

(SEQ ID NO: 14)
ITTTTMQFNPSGLELERPQQLFPQWQPLPQQPPFLQQEPEQPYPQQQPL
PQQQPFPQQPQLPHQHQFPQQLPQQQFPQQMPLQPQQQFPQQMPLQPQQ
QPQFPQQKPFGQYQQPLTQQPYPQQQPLAQQQPSIEEQHQLNLCKEFLL
QQCTLDEKVPLLQSVISFLRPHISQQNSCQLKRQQCCQQLANINEQSRC
PAIQTIVHAIVMQQQVQQQVGHGFVQSQLQQLGQGMPIQLQQQPGQAFV
LPQQQAQFKVVGSLVIQTLPMLCNVHVPPYCSPFGSMATGSGGQ

An exemplary amino acid sequence of a B1-hordein polypeptide from barley is available as UniProt Entry P06470 (e.g., available at the website: www[dot]uniprot[dot]org/uniprot/P06470), provided herein in SEQ ID NO: 15.

(SEQ ID NO: 15)
MKTFLIFALLAIAATSTIAQQQPFPQQPIPQQPQPYPQQPQPYPQQPFP
PQQPFPQQPVPQQPQPYPQQPFPPQQPFPQQPPFWQQKPFPQQPPFGLQ
QPILSQQQPCTPQQTPLPQGQLYQTLLQLQIQYVHPSILQQLNPCKVFL
QQQCSPVPVPQRIARSQMLQQSSCHVLQQQCCQQLPQIPEQFRHEAIRA
IVYSIFLQEQPQQLVEGVSQPQQQLWPQQVGQCSFQQPQPQQVGQQQQV
PQSAFLQPHQIAQLEATTSIALRTLPMMCSVNVPLYRILRGVGPSVGV

An exemplary amino acid sequence of a 75 k gamma secalin polypeptide from rye is available as UniProt Entry H6ULI8 (e.g., available at the website: www[dot]uniprot[dot]org/uniprot/H6ULI8), provided herein in SEQ ID NO: 16.

(SEQ ID NO: 16)
MKTLLMLAILAMATTIATANMQVNPSGQVQRPQQQPFPQPQQSSPQQPQ
QPFPQQPQQPVPQQSQQSFPQTQQPQQPLPQPQQPQQLFPQPQQSSPQQ
SQQVTSQPQQPFPQAQPPQQSCPQSQQPYPQEPQQLFPQSQQPQQPFPQ
PQQPQQPFPQPQPQTQQSIPQPQQPFPQYQEPFPQVHQPQQPSPQQQQP
SIQLSLQQQLNPCKNVLLQQCSPVALVSSLRSKIFPQSECQVMQQQCCQ
QLAQIPQQLQCAAIHSVVHAIIMQQEQREGVQILLPQSHQQHVGQGALA
QVQGIIQPQQLSQLEVVRSLVLQNLPTMCNIYVPRQCSTIQAPFASIVT
GIVGH

An exemplary amino acid sequence of an avenin-3 polypeptide from oat is available as UniProt Entry P80356 (e.g., available at the website: www[dot]uniprot[dot]org/uniprot/P80356), provided herein in SEQ ID NO: 17.

(SEQ ID NO: 17)
MKTFLIFALLAMAATMATAQFDPSEQYQPYPEQQQPILQQQQMLLQQQQ
QMLLQQQPLLQVLQQQLNPCRQFLVQQCSPVAVVPFLRSQILQQSSCQV
MRQQCCRQLEQIPEQLRCPAIHSVVQAIIMQQQQFFQPQMQQQFFQPQM
QQVTQGIFQPQMQQVTQGIFQPQLQQVTQGIFQPQMQGQIEGMRAFALQ
ALPAMCDVYVPPHCPVATAPLGGF

An exemplary amino acid sequence of an avenin-E polypeptide from oat is available as UniProt Entry Q09114 (e.g., available at the website: www[dot]uniprot[dot]org/uniprot/Q09114), provided herein in SEQ ID NO: 18.

(SEQ ID NO: 18)
TTTVQYNPSEQYQPYPEQQEPFVQQQPFVQQQQQPFVQQQQMFLQPLLQ
QQLNPCKQFLVQQCSPVAVVPFLRSQILRQAICQVARQQCCRQLAQIPE
QLRCPAIHSVVQAIILQQQQQQQFFQPQLQQQVFQPQLQQVFNQPQQQA
QFEGMRAFALQALPAMCDVYVPPQCPVATAPLGGF

### Methods for Generating Modified Gluten Polypeptides

In some aspects, provided herein are methods for producing modified gluten polypeptides with a reduced inflammatory potential, wherein the modified gluten polypeptides comprise a modified gluten polypeptide amino acid sequence.

In some embodiments, the methods comprise providing an amino acid sequence of a gluten polypeptide. In some embodiments, the amino acid sequence of a gluten polypeptide comprises an amino acid sequence of any gluten polypeptide known in the art and/or provided herein. In some embodiments, the amino acid sequence of a gluten polypeptide comprises an amino acid sequence of a gluten polypeptide from wheat, rye, barley, triticale, or oat. In some embodiments, the amino acid sequence of a gluten polypeptide comprises an amino acid sequence of any gluten-related polypeptide known in the art and/or provided herein, such as an amino acid sequence of a hordein polypeptide or a secalin polypeptide. In some embodiments, the amino acid sequence of a gluten polypeptide comprises an amino acid sequence of a gliadin polypeptide, e.g., from wheat, rye, barley, triticale or oat. In some embodiments, the amino acid sequence of a gluten polypeptide comprises an amino acid sequence of an α-gliadin polypeptide, a β-gliadin polypeptide, an α/β-gliadin polypeptide, a γ-gliadin polypeptide, or an ω-gliadin polypeptide. In some embodiments, the amino acid sequence of a gluten polypeptide comprises an amino acid sequence of an α/β-gliadin A-I polypeptide, an α/β-gliadin A-II polypeptide, an α/β-gliadin A-III polypeptide, an α/β-gliadin A-IV polypeptide, or an α/β-gliadin A-V polypeptide.

In some embodiments, the amino acid sequence of a gluten polypeptide comprises the amino acid sequence of an α/β-gliadin A-I polypeptide. In some embodiments, the amino acid sequence of a gluten polypeptide comprises the amino acid sequence of SEQ ID NO: 1, or an amino acid sequence having at least about 85%, at least about 90%, at least about 95%, at least about 96%, at least about 97%, at least about 98%, at least about 99%, or 100% sequence identity to the amino acid sequence of SEQ ID NO: 1. In some embodiments, the amino acid sequence of a gluten polypeptide comprises the amino acid sequence of UniProt Entry P04721 (e.g., available at the website: www[dot]uniprot[dot]org/uniprot/P04721), or an amino acid sequence having at least about 85%, at least about 90%, at least about 95%, at least about 96%, at least about 97%, at least about 98%, at least about 99%, or 100% sequence identity to the amino acid sequence of UniProt Entry P04721.

In some embodiments, the amino acid sequence of a gluten polypeptide comprises the amino acid sequence of an α/β-gliadin A-II polypeptide. In some embodiments, the amino acid sequence of a gluten polypeptide comprises the amino acid sequence of SEQ ID NO: 2, or an amino acid sequence having at least about 85%, at least about 90%, at least about 95%, at least about 96%, at least about 97%, at least about 98%, at least about 99%, or 100% sequence identity to the amino acid sequence of SEQ ID NO: 2. In some embodiments, the amino acid sequence of a gluten polypeptide comprises the amino acid sequence of UniProt Entry P04722 (e.g., available at the website: www[dot]uniprot[dot]org/uniprot/P04722), or an amino acid sequence having at least about 85%, at least about 90%, at least about 95%, at least about 96%, at least about 97%, at least about 98%, at least about 99%, or 100% sequence identity to the amino acid sequence of UniProt Entry P04722.

In some embodiments, the amino acid sequence of a gluten polypeptide comprises the amino acid sequence of an α/β-gliadin A-III polypeptide. In some embodiments, the amino acid sequence of a gluten polypeptide comprises the amino acid sequence of SEQ ID NO: 3, or an amino acid sequence having at least about 85%, at least about 90%, at least about 95%, at least about 96%, at least about 97%, at least about 98%, at least about 99%, or 100% sequence identity to the amino acid sequence of SEQ ID NO: 3. In some embodiments, the amino acid sequence of a gluten polypeptide comprises the amino acid sequence of UniProt Entry P04723 (e.g., available at the website: www[dot]uniprot[dot]org/uniprot/P04723), or an amino acid sequence having at least about 85%, at least about 90%, at least about 95%, at least about 96%, at least about 97%, at least about 98%, at least about 99%, or 100% sequence identity to the amino acid sequence of UniProt Entry P04723.

In some embodiments, the amino acid sequence of a gluten polypeptide comprises the amino acid sequence of an α/β-gliadin A-IV polypeptide. In some embodiments, the amino acid sequence of a gluten polypeptide comprises the amino acid sequence of SEQ ID NO: 4, or an amino acid sequence having at least about 85%, at least about 90%, at least about 95%, at least about 96%, at least about 97%, at least about 98%, at least about 99%, or 100% sequence identity to the amino acid sequence of SEQ ID NO: 4. In some embodiments, the amino acid sequence of a gluten polypeptide comprises the amino acid sequence of UniProt Entry P04724 (e.g., available at the website: www[dot]uniprot[dot]org/uniprot/P04724), or an amino acid sequence having at least about 85%, at least about 90%, at least about 95%, at least about 96%, at least about 97%, at least about 98%, at least about 99%, or 100% sequence identity to the amino acid sequence of UniProt Entry P04724.

In some embodiments, the amino acid sequence of a gluten polypeptide comprises the amino acid sequence of an α/β-gliadin A-V polypeptide. In some embodiments, the amino acid sequence of a gluten polypeptide comprises the amino acid sequence of SEQ ID NO: 5, or an amino acid sequence having at least about 85%, at least about 90%, at least about 95%, at least about 96%, at least about 97%, at least about 98%, at least about 99%, or 100% sequence identity to the amino acid sequence of SEQ ID NO: 5. In some embodiments, the amino acid sequence of a gluten polypeptide comprises the amino acid sequence of UniProt Entry P04725 (e.g., available at the website: www[dot]uniprot[dot]org/uniprot/P04725), or an amino acid sequence having at least about 85%, at least about 90%, at least about 95%, at least about 96%, at least about 97%, at least about 98%, at least about 99%, or 100% sequence identity to the amino acid sequence of UniProt Entry P04725.

In some embodiments, the amino acid sequence of a gluten polypeptide comprises an amino acid sequence of a glutenin polypeptide, e.g. from wheat, rye, barley, triticale or oat. In some embodiments, the amino acid sequence of a gluten polypeptide comprises an amino acid sequence of a high-molecular-weight (HMW) subunit polypeptide, or of a low-molecular-weight (LMW) subunit polypeptide.

In some embodiments, the amino acid sequence of a gluten polypeptide comprises the amino acid sequence of a high molecular weight subunit 12 glutenin polypeptide. In some embodiments, the amino acid sequence of a gluten polypeptide comprises the amino acid sequence of SEQ ID NO: 6, or an amino acid sequence having at least about 85%, at least about 90%, at least about 95%, at least about 96%, at least about 97%, at least about 98%, at least about 99%, or 100% sequence identity to the amino acid sequence of SEQ ID NO: 6. In some embodiments, the amino acid sequence of a gluten polypeptide comprises the amino acid sequence of UniProt Entry P08488 (e.g., available at the website: www[dot]uniprot[dot]org/uniprot/P08488), or an amino acid sequence having at least about 85%, at least about 90%, at least about 95%, at least about 96%, at least about 97%, at least about 98%, at least about 99%, or 100% sequence identity to the amino acid sequence of UniProt Entry P08488.

In some embodiments, the amino acid sequence of a gluten-related polypeptide comprises the amino acid sequence of a γ-hordein-1 polypeptide. In some embodiments, the amino acid sequence of a gluten-related polypeptide comprises the amino acid sequence of SEQ ID NO: 13, or an amino acid sequence having at least about 85%, at least about 90%, at least about 95%, at least about 96%, at least about 97%, at least about 98%, at least about 99%, or 100% sequence identity to the amino acid sequence of SEQ ID NO: 13. In some embodiments, the amino acid sequence of a gluten-related polypeptide comprises the amino acid sequence of UniProt Entry P17990 (e.g., available at the website: www[dot]uniprot[dot]org/uniprot/P17990), or an amino acid sequence having at least about 85%, at least about 90%, at least about 95%, at least about 96%, at least about 97%, at least about 98%, at least about 99%, or 100% sequence identity to the amino acid sequence of UniProt Entry P17990.

In some embodiments, the amino acid sequence of a gluten-related polypeptide comprises the amino acid sequence of a γ-hordein-3 polypeptide. In some embodiments, the amino acid sequence of a gluten-related polypeptide comprises the amino acid sequence of SEQ ID NO: 14, or an amino acid sequence having at least about 85%, at least about 90%, at least about 95%, at least about 96%, at least about 97%, at least about 98%, at least about 99%, or 100% sequence identity to the amino acid sequence of SEQ ID NO: 14. In some embodiments, the amino acid sequence of a gluten-related polypeptide comprises the amino acid sequence of UniProt Entry P80198 (e.g., available at the website: www[dot]uniprot[dot]org/uniprot/P80198), or an amino acid sequence having at least about 85%, at least about 90%, at least about 95%, at least about 96%, at least about 97%, at least about 98%, at least about 99%, or 100% sequence identity to the amino acid sequence of UniProt Entry P80198.

In some embodiments, the amino acid sequence of a gluten-related polypeptide comprises the amino acid sequence of a B1-hordein polypeptide. In some embodiments, the amino acid sequence of a gluten-related polypeptide comprises the amino acid sequence of SEQ ID NO: 15, or an amino acid sequence having at least about 85%, at least about 90%, at least about 95%, at least about 96%, at least about 97%, at least about 98%, at least about 99%, or 100% sequence identity to the amino acid sequence of SEQ ID NO: 15. In some embodiments, the amino acid sequence of a gluten-related polypeptide comprises the amino acid sequence of UniProt Entry P06470 (e.g., available at the website: www[dot]uniprot[dot]org/uniprot/P06470), or an amino acid sequence having at least about 85%, at least about 90%, at least about 95%, at least about 96%, at least about 97%, at least about 98%, at least about 99%, or 100% sequence identity to the amino acid sequence of UniProt Entry P06470.

In some embodiments, the amino acid sequence of a gluten-related polypeptide comprises the amino acid sequence of a 75 k gamma secalin polypeptide. In some embodiments, the amino acid sequence of a gluten-related polypeptide comprises the amino acid sequence of SEQ ID NO: 16, or an amino acid sequence having at least about 85%, at least about 90%, at least about 95%, at least about 96%, at least about 97%, at least about 98%, at least about 99%, or 100% sequence identity to the amino acid sequence of SEQ ID NO: 16. In some embodiments, the amino acid sequence of a gluten-related polypeptide comprises the amino acid sequence of UniProt Entry H6ULI8 (e.g., available at the website: www[dot]uniprot[dot]org/uniprot/H6ULI8), or an amino acid sequence having at least about 85%, at least about 90%, at least about 95%, at least about 96%, at least about 97%, at least about 98%, at least about 99%, or 100% sequence identity to the amino acid sequence of UniProt Entry H6ULI8.

In some embodiments, the amino acid sequence of a gluten-related polypeptide comprises the amino acid sequence of an avenin-3 polypeptide. In some embodiments, the amino acid sequence of a gluten-related polypeptide comprises the amino acid sequence of SEQ ID NO: 17, or an amino acid sequence having at least about 85%, at least about 90%, at least about 95%, at least about 96%, at least about 97%, at least about 98%, at least about 99%, or 100% sequence identity to the amino acid sequence of SEQ ID NO: 17. In some embodiments, the amino acid sequence of a gluten-related polypeptide comprises the amino acid sequence of UniProt Entry P80356 (e.g., available at the website: www[dot]uniprot[dot]org/uniprot/P80356), or an amino acid sequence having at least about 85%, at least about 90%, at least about 95%, at least about 96%, at least about 97%, at least about 98%, at least about 99%, or 100% sequence identity to the amino acid sequence of UniProt Entry P80356.

In some embodiments, the amino acid sequence of a gluten-related polypeptide comprises the amino acid sequence of an avenin-E polypeptide. In some embodiments, the amino acid sequence of a gluten-related polypeptide comprises the amino acid sequence of SEQ ID NO: 18, or an amino acid sequence having at least about 85%, at least about 90%, at least about 95%, at least about 96%, at least about 97%, at least about 98%, at least about 99%, or 100% sequence identity to the amino acid sequence of SEQ ID NO: 18. In some embodiments, the amino acid sequence of a gluten-related polypeptide comprises the amino acid sequence of UniProt Entry Q09114 (e.g., available at the website: www[dot]uniprot[dot]org/uniprot/Q09114), or an amino acid sequence having at least about 85%, at least about 90%, at least about 95%, at least about 96%, at least about 97%, at least about 98%, at least about 99%, or 100% sequence identity to the amino acid sequence of UniProt Entry Q09114.

### Inflammatory Amino Acid Sequences in Gluten (ISGs)

Without wishing to be bound by theory, it is believed that the inflammation caused by ingestion of gluten polypeptides by certain individuals is due, at least in part, to the presence of inflammatory amino acid sequences in gluten (ISGs). The ability of ISGs in gluten polypeptides to cause inflammation in an individual, e.g., mediated by the innate immune system, may be caused, at least in part, by any one or more of: the ability of an ISG to permeabilize cell membranes; the ability of an ISG to organize nucleic acids, such as double stranded DNA (dsDNA), double stranded RNA (dsRNA), single-stranded DNA (ssDNA) or single-stranded RNA (ssRNA), into nanocrystalline structures with inter-ligand spacing conducive to toll-like receptor (TLR) activation; the ability of an ISG to remodel cell membranes; the ability of an ISG to access endosomal compartments in cells; the ability of an ISG to amplify and/or promote the activation of TLRs; or the ability of an ISG to self-assemble into amyloidal or protofibril structures to activate formyl peptide receptor-like 1 (FPRL1) and/or formyl peptide receptor 2 (FPR2).

In some embodiments, the methods provided herein comprise identifying and/or selecting an inflammatory amino acid sequence in gluten (ISG) in the amino acid sequence of a gluten polypeptide.

In some embodiments, the methods comprise identifying and/or selecting one or more ISGs in the amino acid sequence of any gluten polypeptide known in the art or provided herein. In some embodiments, the methods comprise identifying and/or selecting one or more ISGs in the amino acid sequence of a gluten polypeptide from wheat, rye, barley, triticale or oat. In some embodiments, the methods comprise identifying and/or selecting one or more ISGs in the amino acid sequence of any gluten-related polypeptide known in the art and/or provided herein (e.g., a hordein or a secalin polypeptide). In some embodiments, the identified or selected ISG comprises an amino acid sequence capable of permeabilizing cell membranes. Alternatively or additionally, the identified or selected ISG comprises an amino acid sequence capable of organizing nucleic acids, such as dsDNA, dsRNA, ssDNA, or ssRNA, into nanocrystalline structures with inter-ligand spacing conducive to toll-like receptor (TLR) activation. Alternatively or additionally, the identified or selected ISG comprises an amino acid sequence capable of remodeling cell membranes. Alternatively or additionally, the identified or selected ISG comprises an amino acid sequence capable of accessing endosomal compartments in cells. Alternatively or additionally, the identified or selected ISG comprises an amino acid sequence capable of amplifying and/or promoting the activation of TLRs. Alternatively or additionally, the identified or selected ISG comprises an amino acid sequence capable of self-assembling into amyloidal or protofibril structures to activate formyl peptide receptor-like 1 (FPRL1) and/or formyl peptide receptor 2 (FPR2).

In some embodiments, the methods comprise analyzing the amino acid sequence of a gluten polypeptide or a gluten-related polypeptide to identify one or more embedded ISGs. In some embodiments, the amino acid sequence of a gluten polypeptide or a gluten-related polypeptide may be analyzed using any suitable method known in the art. In some embodiments, the amino acid sequence of a gluten polypeptide or a gluten-related polypeptide is analyzed using a machine learning algorithm. In an exemplary machine learning algorithm, a moving window of variable amino acid length is used to identify single peptide sequences for further evaluation and/or scoring. In some embodiments, the identified peptide sequences are scored individually, e.g., using a machine learning framework. In some embodiments, the scoring comprises one or more auto-encoders and/or one or more classifiers. In some embodiments, the one or more classifiers are trained to identify peptide sequences with a high probability of possessing immunomodulatory activity, e.g., the ability of an ISG to permeabilize cell membranes; the ability of an ISG to organize nucleic acids, such as dsDNA, dsRNA, ssDNA, or ssRNA, into nanocrystalline structures with inter-ligand spacing conducive to toll-like receptor (TLR) activation; the ability of an ISG to remodel cell membranes; the ability of an ISG to access endosomal compartments in cells; the ability of an ISG to amplify and/or promote the activation of TLRs; and/or the ability of an ISG to self-assemble into amyloidal or protofibril structures to activate formyl peptide receptor-like 1 (FPRL1) and/or formyl peptide receptor 2 (FPR2). In some embodiments, the one or more classifiers are trained using a curated, binary labeled dataset comprising peptides possessing immunomodulatory activity and a decoy set of peptides. In some embodiments, the one or more classifiers are vetted using experimental data, such as, without limitation, experimental data from X-ray diffraction, in vitro trafficking assays, microscopy, immune activation experiments, and any combination thereof. In some embodiments, a candidate ISG is identified as a peptide sequence based on the output scores of the one or more classifiers. In some embodiments, a candidate ISG is identified as a peptide sequence having the highest output scores of the one or more classifiers, e.g., as compared to other peptide sequences. In some embodiments, the methods further comprise evaluating candidate ISGs to identify the core ISG sequence, e.g., by analyzing peptide sequences from overlapping windows and/or the extent of the amino acid sequence toward the N- and C-termini of the ISG.

In some embodiments, the methods comprise identifying and/or selecting one or more ISGs in the amino acid sequence of a gliadin polypeptide, e.g., from wheat, rye, barley, triticale or oat. In some embodiments, the methods comprise identifying and/or selecting one or more ISGs in the amino acid sequence of an α-gliadin polypeptide, a β-gliadin polypeptide, an α/β-gliadin polypeptide, a 7-gliadin polypeptide, or an ω-gliadin polypeptide. In some embodiments, the methods comprise identifying and/or selecting one or more ISGs in the amino acid sequence of an α/β-gliadin A-I polypeptide, an α/β-gliadin A-II polypeptide, an α/β-gliadin A-III polypeptide, an α/β-gliadin A-IV polypeptide, or an α/β-gliadin A-V polypeptide.

In some embodiments, the methods comprise identifying and/or selecting one or more ISGs in the amino acid sequence of an α/β-gliadin A-I polypeptide. In some embodiments, the methods comprise identifying and/or selecting one or more ISGs in the amino acid sequence of SEQ ID NO: 1, or an amino acid sequence having at least about 85%, at least about 90%, at least about 95%, at least about 96%, at least about 97%, at least about 98%, at least about 99%, or 100% sequence identity to the amino acid sequence of SEQ ID NO: 1. In some embodiments, the methods comprise identifying and/or selecting one or more ISGs in the amino acid sequence of UniProt Entry P04721 (e.g., available at the website: www[dot]uniprot[dot]org/uniprot/P04721), or an amino acid sequence having at least about 85%, at least about 90%, at least about 95%, at least about 96%, at least about 97%, at least about 98%, at least about 99%, or 100% sequence identity to the amino acid sequence of UniProt Entry P04721. In some embodiments, the identified and/or selected ISG comprises the amino acid sequence of amino acid residues 159-206 of SEQ ID NO: 1, or of amino acid residues corresponding to amino acid residues 159-206 of an α/β-gliadin A-I polypeptide. In some embodiments, the identified and/or selected ISG comprises the amino acid sequence of amino acid residues 159-206 of the amino acid sequence of UniProt Entry P04721. In some embodiments, the identified and/or selected ISG comprises the amino acid sequence of SEQ ID NO: 7, or an amino acid sequence having at least about 85%, at least about 90%, at least about 95%, at least about 96%, at least about 97%, at least about 98%, at least about 99%, or 100% sequence identity to the amino acid sequence of SEQ ID NO: 7.

In some embodiments, the methods comprise identifying and/or selecting one or more ISGs in the amino acid sequence of an α/β-gliadin A-II polypeptide. In some embodiments, the methods comprise identifying and/or selecting one or more ISGs in the amino acid sequence of SEQ ID NO: 2, or an amino acid sequence having at least about 85%, at least about 90%, at least about 95%, at least about 96%, at least about 97%, at least about 98%, at least about 99%, or 100% sequence identity to the amino acid sequence of SEQ ID NO: 2. In some embodiments, the methods comprise identifying and/or selecting one or more ISGs in the amino acid sequence of UniProt Entry P04722 (e.g., available at the website: www[dot]uniprot[dot]org/uniprot/P04722), or an amino acid sequence having at least about 85%, at least about 90%, at least about 95%, at least about 96%, at least about 97%, at least about 98%, at least about 99%, or 100% sequence identity to the amino acid sequence of UniProt Entry P04722. In some embodiments, the identified and/or selected ISG comprises the amino acid sequence of amino acid residues 155-207 of SEQ ID NO: 2, or of amino acid residues corresponding to amino acid residues 155-207 of an α/β-gliadin A-II polypeptide. In some embodiments, the identified and/or selected ISG comprises the amino acid sequence of amino acid residues 155-207 of the amino acid sequence of UniProt Entry P04722. In some embodiments, the identified and/or selected ISG comprises the amino acid sequence of SEQ ID NO: 8, or an amino acid sequence having at least about 85%, at least about 90%, at least about 95%, at least about 96%, at least about 97%, at least about 98%, at least about 99%, or 100% sequence identity to the amino acid sequence of SEQ ID NO: 8.

In some embodiments, the methods comprise identifying and/or selecting one or more ISGs in the amino acid sequence of an α/β-gliadin A-III polypeptide. In some embodiments, the methods comprise identifying and/or selecting one or more ISGs in the amino acid sequence of SEQ ID NO: 3, or an amino acid sequence having at least about 85%, at least about 90%, at least about 95%, at least about 96%, at least about 97%, at least about 98%, at least about 99%, or 100% sequence identity to the amino acid sequence of SEQ ID NO: 3. In some embodiments, the methods comprise identifying and/or selecting one or more ISGs in the amino acid sequence of UniProt Entry P04723 (e.g., available at the website: www[dot]uniprot[dot]org/uniprot/P04723), or an amino acid sequence having at least about 85%, at least about 90%, at least about 95%, at least about 96%, at least about 97%, at least about 98%, at least about 99%, or 100% sequence identity to the amino acid sequence of UniProt Entry P04723. In some embodiments, the identified and/or selected ISG comprises the amino acid sequence of amino acid residues 152-199 of SEQ ID NO: 3, or of amino acid residues corresponding to amino acid residues 152-199 of an α/β-gliadin A-III polypeptide. In some embodiments, the identified and/or selected ISG comprises the amino acid sequence of amino acid residues 152-199 of the amino acid sequence of UniProt Entry P04723. In some embodiments, the identified and/or selected ISG comprises the amino acid sequence of SEQ ID NO: 9, or an amino acid sequence having at least about 85%, at least about 90%, at least about 95%, at least about 96%, at least about 97%, at least about 98%, at least about 99%, or 100% sequence identity to the amino acid sequence of SEQ ID NO: 9.

In some embodiments, the methods comprise identifying and/or selecting one or more ISGs in the amino acid sequence of an α/β-gliadin A-IV polypeptide. In some embodiments, the methods comprise identifying and/or selecting one or more ISGs in the amino acid sequence of SEQ ID NO: 4, or an amino acid sequence having at least about 85%, at least about 90%, at least about 95%, at least about 96%, at least about 97%, at least about 98%, at least about 99%, or 100% sequence identity to the amino acid sequence of SEQ ID NO: 4. In some embodiments, the methods comprise identifying and/or selecting one or more ISGs in the amino acid sequence of UniProt Entry P04724 (e.g., available at the website: www[dot]uniprot[dot]org/uniprot/P04724), or an amino acid sequence having at least about 85%, at least about 90%, at least about 95%, at least about 96%, at least about 97%, at least about 98%, at least about 99%, or 100% sequence identity to the amino acid sequence of UniProt Entry P04724. In some embodiments, the identified and/or selected ISG comprises the amino acid sequence of amino acid residues 167-215 of SEQ ID NO: 4, or of amino acid residues corresponding to amino acid residues 167-215 of an α/β-gliadin A-IV polypeptide. In some embodiments, the identified and/or selected ISG comprises the amino acid sequence of amino acid residues 167-215 of the amino acid sequence of UniProt Entry P04724. In some embodiments, the identified and/or selected ISG comprises the amino acid sequence of SEQ ID NO: 10, or an amino acid sequence having at least about 85%, at least about 90%, at least about 95%, at least about 96%, at least about 97%, at least about 98%, at least about 99%, or 100% sequence identity to the amino acid sequence of SEQ ID NO: 10.

In some embodiments, the methods comprise identifying and/or selecting one or more ISGs in the amino acid sequence of an α/β-gliadin A-V polypeptide. In some embodiments, the methods comprise identifying and/or selecting one or more ISGs in the amino acid sequence of SEQ ID NO: 5, or an amino acid sequence having at least about 85%, at least about 90%, at least about 95%, at least about 96%, at least about 97%, at least about 98%, at least about 99%, or 100% sequence identity to the amino acid sequence of SEQ ID NO: 5. In some embodiments, the methods comprise identifying and/or selecting one or more ISGs in the amino acid sequence of UniProt Entry P04725 (e.g., available at the website: www[dot]uniprot[dot]org/uniprot/P04725), or an amino acid sequence having at least about 85%, at least about 90%, at least about 95%, at least about 96%, at least about 97%, at least about 98%, at least about 99%, or 100% sequence identity to the amino acid sequence of UniProt Entry P04725. In some embodiments, the identified and/or selected ISG comprises the amino acid sequence of amino acid residues 164-223 of SEQ ID NO: 5, or of amino acid residues corresponding to amino acid residues 164-223 of an α/β-gliadin A-V polypeptide. In some embodiments, the identified and/or selected ISG comprises the amino acid sequence of amino acid residues 164-223 of the amino acid sequence of UniProt Entry P04725. In some embodiments, the identified and/or selected ISG comprises the amino acid sequence of SEQ ID NO: 11, or an amino acid sequence having at least about 85%, at least about 90%, at least about 95%, at least about 96%, at least about 97%, at least about 98%, at least about 99%, or 100% sequence identity to the amino acid sequence of SEQ ID NO: 11.

In some embodiments, the methods comprise identifying and/or selecting one or more ISGs in the amino acid sequence of a glutenin polypeptide, e.g. from wheat, rye, barley, triticale or oat. In some embodiments, the methods comprise identifying and/or selecting one or more ISGs in the amino acid sequence of a high-molecular-weight (HMW) subunit polypeptide, or of a low-molecular-weight (LMW) subunit polypeptide. In some embodiments, the methods comprise identifying and/or selecting one or more ISGs in the amino acid sequence of a high molecular weight subunit 12 glutenin polypeptide. In some embodiments, the methods comprise identifying and/or selecting one or more ISGs in the amino acid sequence of SEQ ID NO: 6, or an amino acid sequence having at least about 85%, at least about 90%, at least about 95%, at least about 96%, at least about 97%, at least about 98%, at least about 99%, or 100% sequence identity to the amino acid sequence of SEQ ID NO: 6. In some embodiments, the methods comprise identifying and/or selecting one or more ISGs in the amino acid sequence of UniProt Entry P08488 (e.g., available at the website: www[dot]uniprot[dot]org/uniprot/P08488), or an amino acid sequence having at least about 85%, at least about 90%, at least about 95%, at least about 96%, at least about 97%, at least about 98%, at least about 99%, or 100% sequence identity to the amino acid sequence of UniProt Entry P08488. In some embodiments, the identified and/or selected ISG comprises the amino acid sequence of amino acid residues 45-86 of SEQ ID NO: 6, or of amino acid residues corresponding to amino acid residues 45-86 of a high molecular weight subunit 12 glutenin polypeptide. In some embodiments, the identified and/or selected ISG comprises the amino acid sequence of amino acid residues 45-86 of the amino acid sequence of UniProt Entry P08488. In some embodiments, the identified and/or selected ISG comprises the amino acid sequence of SEQ ID NO: 12, or an amino acid sequence having at least about 85%, at least about 90%, at least about 95%, at least about 96%, at least about 97%, at least about 98%, at least about 99%, or 100% sequence identity to the amino acid sequence of SEQ ID NO: 12.

In some embodiments, the methods comprise identifying and/or selecting one or more ISGs in the amino acid sequence of a gluten-related polypeptide, e.g. from wheat, rye, barley, triticale or oat.

In some embodiments, the methods comprise identifying and/or selecting one or more ISGs in the amino acid sequence of a γ-hordein-1 polypeptide. In some embodiments, the methods comprise identifying and/or selecting one or more ISGs in the amino acid sequence of SEQ ID NO: 13, or an amino acid sequence having at least about 85%, at least about 90%, at least about 95%, at least about 96%, at least about 97%, at least about 98%, at least about 99%, or 100% sequence identity to the amino acid sequence of SEQ ID NO: 13. In some embodiments, the methods comprise identifying and/or selecting one or more ISGs in the amino acid sequence of UniProt Entry P17990 (e.g., available at the website: www[dot]uniprot[dot]org/uniprot/P17990), or an amino acid sequence having at least about 85%, at least about 90%, at least about 95%, at least about 96%, at least about 97%, at least about 98%, at least about 99%, or 100% sequence identity to the amino acid sequence of UniProt Entry P17990. In some embodiments, the identified and/or selected ISG comprises the amino acid sequence of amino acid residues 182-235 of SEQ ID NO: 13, or of amino acid residues corresponding to amino acid residues 182-235 of a γ-hordein-1 polypeptide. In some embodiments, the identified and/or selected ISG comprises the amino acid sequence of amino acid residues 182-235 of the amino acid sequence of UniProt Entry P17990. In some embodiments, the identified and/or selected ISG comprises the amino acid sequence of SEQ ID NO: 24, or an amino acid sequence having at least about 85%, at least about 90%, at least about 95%, at least about 96%, at least about 97%, at least about 98%, at least about 99%, or 100% sequence identity to the amino acid sequence of SEQ ID NO: 24.

In some embodiments, the methods comprise identifying and/or selecting one or more ISGs in the amino acid sequence of a γ-hordein-3 polypeptide. In some embodiments, the methods comprise identifying and/or selecting one or more ISGs in the amino acid sequence of SEQ ID NO: 14, or an amino acid sequence having at least about 85%, at least about 90%, at least about 95%, at least about 96%, at least about 97%, at least about 98%, at least about 99%, or 100% sequence identity to the amino acid sequence of SEQ ID NO: 14. In some embodiments, the methods comprise identifying and/or selecting one or more ISGs in the amino acid sequence of UniProt Entry P80198 (e.g., available at the website: www[dot]uniprot[dot]org/uniprot/P80198), or an amino acid sequence having at least about 85%, at least about 90%, at least about 95%, at least about 96%, at least about 97%, at least about 98%, at least about 99%, or 100% sequence identity to the amino acid sequence of UniProt Entry P80198. In some embodiments, the identified and/or selected ISG comprises the amino acid sequence of amino acid residues 165-214 of SEQ ID NO: 14, or of amino acid residues corresponding to amino acid residues 165-214 of a 7-hordein-3 polypeptide. In some embodiments, the identified and/or selected ISG comprises the amino acid sequence of amino acid residues 165-214 of the amino acid sequence of UniProt Entry P80198. In some embodiments, the identified and/or selected ISG comprises the amino acid sequence of SEQ ID NO: 25, or an amino acid sequence having at least about 85%, at least about 90%, at least about 95%, at least about 96%, at least about 97%, at least about 98%, at least about 99%, or 100% sequence identity to the amino acid sequence of SEQ ID NO: 25.

In some embodiments, the methods comprise identifying and/or selecting one or more ISGs in the amino acid sequence of a B1-hordein polypeptide. In some embodiments, the methods comprise identifying and/or selecting one or more ISGs in the amino acid sequence of SEQ ID NO: 15, or an amino acid sequence having at least about 85%, at least about 90%, at least about 95%, at least about 96%, at least about 97%, at least about 98%, at least about 99%, or 100% sequence identity to the amino acid sequence of SEQ ID NO: 15. In some embodiments, the methods comprise identifying and/or selecting one or more ISGs in the amino acid sequence of UniProt Entry P06470 (e.g., available at the website: www[dot]uniprot[dot]org/uniprot/P06470), or an amino acid sequence having at least about 85%, at least about 90%, at least about 95%, at least about 96%, at least about 97%, at least about 98%, at least about 99%, or 100% sequence identity to the amino acid sequence of UniProt Entry P06470. In some embodiments, the identified and/or selected ISG comprises the amino acid sequence of amino acid residues 160-208 of SEQ ID NO: 15, or of amino acid residues corresponding to amino acid residues 160-208 of a B1-hordein polypeptide. In some embodiments, the identified and/or selected ISG comprises the amino acid sequence of amino acid residues 160-208 of the amino acid sequence of UniProt Entry P06470. In some embodiments, the identified and/or selected ISG comprises the amino acid sequence of SEQ ID NO: 22, or an amino acid sequence having at least about 85%, at least about 90%, at least about 95%, at least about 96%, at least about 97%, at least about 98%, at least about 99%, or 100% sequence identity to the amino acid sequence of SEQ ID NO: 22.

In some embodiments, the methods comprise identifying and/or selecting one or more ISGs in the amino acid sequence of a 75 k gamma secalin polypeptide. In some embodiments, the methods comprise identifying and/or selecting one or more ISGs in the amino acid sequence of SEQ ID NO: 16, or an amino acid sequence having at least about 85%, at least about 90%, at least about 95%, at least about 96%, at least about 97%, at least about 98%, at least about 99%, or 100% sequence identity to the amino acid sequence of SEQ ID NO: 16. In some embodiments, the methods comprise identifying and/or selecting one or more ISGs in the amino acid sequence of UniProt Entry H6ULI8 (e.g., available at the website: www[dot]uniprot[dot]org/uniprot/H6ULI8), or an amino acid sequence having at least about 85%, at least about 90%, at least about 95%, at least about 96%, at least about 97%, at least about 98%, at least about 99%, or 100% sequence identity to the amino acid sequence of UniProt Entry H6ULI8.

In some embodiments, the methods comprise identifying and/or selecting one or more ISGs in the amino acid sequence of an avenin-3 polypeptide. In some embodiments, the methods comprise identifying and/or selecting one or more ISGs in the amino acid sequence of SEQ ID NO: 17, or an amino acid sequence having at least about 85%, at least about 90%, at least about 95%, at least about 96%, at least about 97%, at least about 98%, at least about 99%, or 100% sequence identity to the amino acid sequence of SEQ ID NO: 17. In some embodiments, the methods comprise identifying and/or selecting one or more ISGs in the amino acid sequence of UniProt Entry P80356 (e.g., available at the website: www[dot]uniprot[dot]org/uniprot/P80356), or an amino acid sequence having at least about 85%, at least about 90%, at least about 95%, at least about 96%, at least about 97%, at least about 98%, at least about 99%, or 100% sequence identity to the amino acid sequence of UniProt Entry P80356. In some embodiments, the identified and/or selected ISG comprises the amino acid sequence of amino acid residues 85-141 of SEQ ID NO: 17, or of amino acid residues corresponding to amino acid residues 85-141 of an avenin-3 polypeptide. In some embodiments, the identified and/or selected ISG comprises the amino acid sequence of amino acid residues 85-141 of the amino acid sequence of UniProt Entry P80356. In some embodiments, the identified and/or selected ISG comprises the amino acid sequence of SEQ ID NO: 27, or an amino acid sequence having at least about 85%, at least about 90%, at least about 95%, at least about 96%, at least about 97%, at least about 98%, at least about 99%, or 100% sequence identity to the amino acid sequence of SEQ ID NO: 27.

In some embodiments, the methods comprise identifying and/or selecting one or more ISGs in the amino acid sequence of an avenin-E polypeptide. In some embodiments, the methods comprise identifying and/or selecting one or more ISGs in the amino acid sequence of SEQ ID NO: 18, or an amino acid sequence having at least about 85%, at least about 90%, at least about 95%, at least about 96%, at least about 97%, at least about 98%, at least about 99%, or 100% sequence identity to the amino acid sequence of SEQ ID NO: 18. In some embodiments, the methods comprise identifying and/or selecting one or more ISGs in the amino acid sequence of UniProt Entry Q09114 (e.g., available at the website: www[dot]uniprot[dot]org/uniprot/Q09114), or an amino acid sequence having at least about 85%, at least about 90%, at least about 95%, at least about 96%, at least about 97%, at least about 98%, at least about 99%, or 100% sequence identity to the amino acid sequence of UniProt Entry Q09114. In some embodiments, the identified and/or selected ISG comprises the amino acid sequence of amino acid residues 71-130 of SEQ ID NO: 18, or of amino acid residues corresponding to amino acid residues 71-130 of an avenin-E polypeptide. In some embodiments, the identified and/or selected ISG comprises the amino acid sequence of amino acid residues 71-130 of the amino acid sequence of UniProt Entry Q09114. In some embodiments, the identified and/or selected ISG comprises the amino acid sequence of SEQ ID NO: 26, or an amino acid sequence having at least about 85%, at least about 90%, at least about 95%, at least about 96%, at least about 97%, at least about 98%, at least about 99%, or 100% sequence identity to the amino acid sequence of SEQ ID NO: 26.

In some embodiments, the methods comprise identifying and/or selecting one or more ISGs in the amino acid sequence of a B3-hordein polypeptide. In some embodiments, the identified and/or selected ISG comprises the amino acid sequence of SEQ ID NO: 23, or an amino acid sequence having at least about 85%, at least about 90%, at least about 95%, at least about 96%, at least about 97%, at least about 98%, at least about 99%, or 100% sequence identity to the amino acid sequence of SEQ ID NO: 23.

In some embodiments, the methods comprise identifying and/or selecting one or more ISGs in the amino acid sequence of a γ-gliadin B-I polypeptide. In some embodiments, the identified and/or selected ISG comprises the amino acid sequence of SEQ ID NO: 19, or an amino acid sequence having at least about 85%, at least about 90%, at least about 95%, at least about 96%, at least about 97%, at least about 98%, at least about 99%, or 100% sequence identity to the amino acid sequence of SEQ ID NO: 19.

In some embodiments, the methods comprise identifying and/or selecting one or more ISGs in the amino acid sequence of a γ-gliadin B-II polypeptide. In some embodiments, the identified and/or selected ISG comprises the amino acid sequence of SEQ ID NO: 20, or an amino acid sequence having at least about 8500, at least about 9000, at least about 9500, at least about 96%, at least about 97%, at least about 98%, at least about 99%, or 100% sequence identity to the amino acid sequence of SEQ ID NO: 20.

In some embodiments, the methods comprise identifying and/or selecting one or more ISGs in the amino acid sequence of a γ-gliadin B-IL polypeptide. In some embodiments, the identified and/or selected ISG comprises the amino acid sequence of SEQ TD NO: 21, or an amino acid sequence having at least about 85%, at least about 90%, at least about 9500 at least about 96%, at least about 97%, at least about 98%, at least about 99%, or 1000% sequence identity to the amino acid sequence of SEQ ID NO: 21.

The amino acid sequences of certain exemplary ISGs of the disclosure are provided in Table 1.

TABLE 1
Exemplary inflammatory amino acid sequences in gluten.

| Description | Sequence | SEQ ID NO |
|---|---|---|
| α/β-gliadin A-I (Triticum | HGKSQVLQQSTYQLLQELCCQHLWQIP | 7 |
| aestivum) ISG | EQSQCQAIHNVVHAIILHQQQ | |
| α/β-gliadin A-II (Triticum | HGSSQVLQESTYQLVQQLCCQQLWQIP | 8 |
| aestivum) ISG | EQSRCQAIHNVVHAIILHQQHHHHQQ | |
| α/β-gliadin A-III (Triticum | HASSQVLQQSSYQQLQQLCCQQLFQIP | 9 |
| aestivum) ISG | EQSRCQAIHNVVHAIILHHHQ | |
| α/β-gliadin A-IV (Triticum | VLQQSTYQLVQQFCCQQLWQIPEQSRC | 10 |
| aestivum) ISG | QAIHNVVHAIILHQQQQQQQQQ | |
| α/β-gliadin A-V (Triticum | HASSQVLQQSTYQLLQQLCCQQLLQIP | 11 |
| aestivum) ISG | EQSQCQAIHNVAHAIIMHQQQQQQQEQ | |
| | KQQLQQ | |
| Glutenin, high molecular weight | QVVDQQLAGRLPWSTGLQMRCCQQLRD | 12 |
| subunit 12 (Triticum aestivum) | VSAKCRSVAVSQVAR | |
| ISG | | |
| γ-gliadin B-I (Triticum aestivum) | LARSQMWQQSSCHVMQQQCCQQLQQIP | 19 |
| ISG | EQSRYEAIRAIIYSIILQEQQQGFVQP | |
| | Q | |
| γ-gliadin B-II (Triticum | SLWSIIWPQSDCQVMRQQCCQQLAQIP | 20 |
| aestivum) ISG | QQLQCAAIHSVVHSIIMQQQQQQQQQQ | |
| | GID | |
| γ-gliadin B-III (Triticum | LARSQMLQQSSCHVMQQQCCQQLPQIP | 21 |
| aestivum) ISG | QQSRYQAIRAIIYSIILQEQQQVQGSI | |
| | QSQ | |
| B1-hordein (Hordeum vulgare) | IARSQMLQQSSCHVLQQQCCQQLPQIP | 22 |
| ISG | EQFRHEAIRAIVYSIFLQEQPQ | |
| B3-hordein (Hordeum vugare) | IARSQMLQQSSCHVLQQQCCQQLPQIP | 23 |
| ISG | EQLRHEAVRAIVYSIVLQEQSL | |
| γ-hordein-1 (Hordeum vugare) | YIWSKIVQQSSCRVMQQQCCLQLAQIP | 24 |
| ISG | EQYKCTAIDSIVHAIFMQQGQRQGVQI | |
| γ-hordein-3 (Hordeum vugare) | FLRPHISQQNSCQLKRQQCCQQLANIN | 25 |
| ISG | EQSRCPAIQTIVHAIVMQQQVQQ | |
| Avenin-E (Avena sativa) ISG | FLRSQILRQAICQVARQQCCRQLAQIP | 26 |
| | EQLRCPAIHSVVQAIILQQQQQQQFFQ | |
| | PQLQQQ | |
| Avenin-3 (Avena sativa) ISG | FLRSQILQQSSCQVMRQQCCRQLEQIP | 27 |
| | EQLRCPAIHSVVQAIIMQQQQFFQPQM | |
| | QQQ | |

In some embodiments, an ISG of the disclosure is a fragment of any ISG described herein or identified and/or selected according to the methods provided herein, so long as the fragment retains the ability to cause inflammation in an individual, and/or retains the ability to permeabilize cell membranes; organize nucleic acids, such as dsDNA, dsRNA, ssDNA or ssRNA, into nanocrystalline structures with inter-ligand spacing conducive to toll-like receptor (TLR) activation; remodel cell membranes; access endosomal compartments in cells; amplify and/or promote the activation of TLRs; and/or self-assemble into amyloidal or protofibril structures to activate formyl peptide receptor-like 1 (FPRL1) and/or formyl peptide receptor 2 (FPR2).

### Alterations of ISG Sequences

In some embodiments, the methods provided herein comprise introducing one or more alterations in an ISG (e.g., an ISG identified or selected according to the methods provided herein) in the amino acid sequence of a gluten polypeptide, thereby generating a modified gluten polypeptide amino acid sequence.

In some embodiments, the one or more alterations include: (i) a substitution of one or more amino acid residues, (ii) a deletion of one or more amino acid residues, and/or (iii) an insertion of one or more amino acid residues.

In some embodiments, the one or more alterations include a substitution of one or more amino acid residues. Amino acids may be grouped according to common side-chain properties:
(1) hydrophobic: Norleucine, Met, Ala, Val, Leu, Ile;
(2) neutral hydrophilic: Cys, Ser, Thr, Asn, Gln;
(3) acidic: Asp, Glu;
(4) basic: His, Lys, Arg;
(5) residues that influence chain orientation: Gly, Pro;
(6) aromatic: Trp, Tyr, Phe.

In some embodiments, the substitution of one or more amino acid residues is a conservative or a non-conservative amino acid substitution. Conservative amino acid substitutions entail exchanging a member of one of the above classes of amino acids for an amino acid within the same class. Non-conservative amino acid substitutions entail exchanging a member of one of the above classes of amino acids for an amino acid in another class. In some embodiments, any of between about 1 and about 5, between about 5 and about 10, between about 10 and about 15, between about 15 and about 20, between about 20 and about 25, between about 25 and about 30, between about 30 and about 35, between about 35 and about 40, between about 40 and about 45, between about 45 and about 50, between about 50 and about 55, between about 55 and about 60, between about 60 and about 65, or between about 65 and about 70 amino acids in an ISG are substituted. In some embodiments, at least 1, at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 21, at least 22, at least 23, at least 24, at least 25, at least 26, at least 27, at least 28, at least 29, at least 30, at least 31, at least 32, at least 33, at least 34, at least 35, at least 36, at least 37, at least 38, at least 39, at least 40, at least 41, at least 42, at least 43, at least 44, at least 45, at least 46, at least 47, at least 48, at least 49, at least 50, at least 51, at least 52, at least 53, at least 54, at least 55, at least 56, at least 57, at least 58, at least 59, at least 60, at least 61, at least 62, at least 63, at least 64, at least 65, at least 66, at least 67, at least 68, at least 69, or at least 70, or more amino acids in an ISG are substituted.

In some embodiments, the one or more alterations include a deletion of one or more amino acid residues in an ISG. In some embodiments, any of between about 1 and about 5, between about 5 and about 10, between about 10 and about 15, between about 15 and about 20, between about 20 and about 25, between about 25 and about 30, between about 30 and about 35, between about 35 and about 40, between about 40 and about 45, between about 45 and about 50, between about 50 and about 55, between about 55 and about 60, between about 60 and about 65, or between about 65 and about 70 amino acids in an ISG are deleted. In some embodiments, at least 1, at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 21, at least 22, at least 23, at least 24, at least 25, at least 26, at least 27, at least 28, at least 29, at least 30, at least 31, at least 32, at least 33, at least 34, at least 35, at least 36, at least 37, at least 38, at least 39, at least 40, at least 41, at least 42, at least 43, at least 44, at least 45, at least 46, at least 47, at least 48, at least 49, at least 50, at least 51, at least 52, at least 53, at least 54, at least 55, at least 56, at least 57, at least 58, at least 59, at least 60, at least 61, at least 62, at least 63, at least 64, at least 65, at least 66, at least 67, at least 68, at least 69, or at least 70, or more amino acids in an ISG are deleted.

In some embodiments, the one or more alterations include an insertion of one or more amino acid residues in an ISG. In some embodiments, any of between about 1 and about 5, between about 5 and about 10, between about 10 and about 15, between about 15 and about 20, between about 20 and about 25, between about 25 and about 30, between about 30 and about 35, between about 35 and about 40, between about 40 and about 45, between about 45 and about 50, between about 50 and about 55, between about 55 and about 60, between about 60 and about 65, or between about 65 and about 70 amino acids are inserted in an ISG. In some embodiments, at least 1, at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 21, at least 22, at least 23, at least 24, at least 25, at least 26, at least 27, at least 28, at least 29, at least 30, at least 31, at least 32, at least 33, at least 34, at least 35, at least 36, at least 37, at least 38, at least 39, at least 40, at least 41, at least 42, at least 43, at least 44, at least 45, at least 46, at least 47, at least 48, at least 49, at least 50, at least 51, at least 52, at least 53, at least 54, at least 55, at least 56, at least 57, at least 58, at least 59, at least 60, at least 61, at least 62, at least 63, at least 64, at least 65, at least 66, at least 67, at least 68, at least 69, or at least 70, or more amino acids are inserted in an ISG.

In some embodiments, the one or more alterations are selected based on one or more classifiers of a machine learning algorithm, such as a machine learning algorithm and/or one or more classifiers described herein in the “Inflammatory Amino Acid Sequences in Gluten (ISGs)” section. In some embodiments, the one or more alterations are selected using an in silico method, wherein single amino acid alterations are serially introduced in the amino acid sequence of an ISG in silico and the output score of the one or more classifiers is assessed after introduction of each alteration, e.g., to determine whether the probability score of immunomodulatory activity outputted by the one or more classifiers is modulated, e.g., reduced or increased. In some embodiments, serial introduction of alterations is stopped when, e.g., a low probability score of immunomodulatory activity is outputted by the one or more classifiers. Alternatively or additionally, one or more alterations, e.g., substitutions or insertions of one or more amino acids, may be selected based on one or more properties, including, without limitation, hydrophobicity (including, but not limited to, changing the hydrophobic moment, the shape of the hydrophobic face, or the choice of aromatic or aliphatic hydrophobes) or alteration of charge (e.g., introduction or elimination of anionic or cationic charge).

### Methods of Producing Modified Gluten Polypeptides

In some embodiments, the methods of the disclosure comprise producing a gluten polypeptide comprising a modified gluten polypeptide amino acid sequence, e.g., comprising one or more alterations in one or more ISGs.

### Recombinant Nucleic Acids, Vectors and Host Cells

In some embodiments, a recombinant nucleic acid molecule comprising a nucleotide sequence encoding a modified gluten polypeptide of the disclosure may be generated. One of skill in the art can readily arrive at one or more nucleotide sequences encoding a modified gluten polypeptide of the disclosure. In some embodiments, a nucleotide sequence encoding a modified gluten polypeptide of the disclosure is optimized (e.g., codon optimized) to improve expression of the modified gluten polypeptide, e.g., in a host cell. In some embodiments, the recombinant nucleic acid molecule is a vector, e.g., an expression vector, comprising a nucleotide sequence encoding a modified gluten polypeptide of the disclosure. In some embodiments, the vector is a plasmid, cosmid or viral vector. The vector can be capable of autonomous replication or it can integrate into a host DNA. Viral vectors include, e.g., replication defective retroviruses, adenoviruses and adeno-associated viruses. In some embodiments, the vector includes one or more regulatory sequences operatively linked to the nucleic acid sequence to be expressed, such as promoters, enhancers and other expression control elements (e.g., polyadenylation signals). In some embodiments, the regulatory sequences direct constitutive expression of the modified gluten polypeptide. In some embodiments, the regulatory sequences direct tissue-specific expression of the modified gluten polypeptide. In some embodiments, the regulatory sequences direct inducible expression of the modified gluten polypeptide (e.g., a promoter regulated by a steroid hormone, by a polypeptide hormone (e.g., by means of a signal transduction pathway), or by a heterologous polypeptide (e.g., the tetracycline-inducible systems, “Tet-On” and “Tet-Off”; see, e.g., Clontech Inc., CA, Gossen and Bujard (1992) Proc. Natl. Acad. Sci. USA 89:5547, and Paillard (1989) Human Gene Therapy 9:983)). The design of the expression vector can depend on such factors as the choice of the host cell to be transformed, the in vitro method to be used, the level of expression desired, and the like.

In some embodiments, a modified gluten polypeptide of the disclosure may be produced using one or more host cells. In some embodiments, the recombinant nucleic acid molecule, e.g., the vector, may be transferred into a host cell. The host cell may be, for example, a prokaryotic cell such as a bacterial cell, a yeast or other fungal cell, an insect cell, a plant cell, or a mammalian cell.

Suitable prokaryotes that may be used as host cells include, but are not limited to, eubacteria, such as Gram-negative or Gram-positive organisms, for example, E. coli. Other suitable prokaryotic host cells include Enterobacter, Erwinia, Klebsiella, Proteus, Salmonella, e.g., Salmonella typhimurium, Serratia, e.g., Serratia marcescans, and Shigella, as well as Bacilli such as B. subtilis and B. lichenformis (e.g., B. lichenformis 4 IP), Pseudomonas such as P. aeruginosa, and Streptomyces.

Suitable plant cells that may be used as host cells include, but are not limited to, cells from any plant family, genus, species, variety, or cultivar described herein. In some embodiments, the host cell is a wheat cell (e.g., any of T. aestivum, T aethiopicum, T araraticum, T. boeoticum, T. carthlicum, T compactum, T. dicoccoides, T. dicoccon, T. durum, T. ispahanicum, T. karamyschevii, T. macha, T. militinae, T. monococcum, T. polonicum, T. spelta, T sphaerococcum, T. timopheevii, T. turanicum, T turgidum, T. urartu, T. vavilovii, or T. zhukovskyi). In some embodiments, the host cell is a wheat hybrid cell. In some embodiments, the host cell is a T. aestivum cell. In some embodiments, the host cell is a wheat and rye hybrid cell. In some embodiments, the host cell is a triticale cell. In some embodiments, the host cell is a rye cell (e.g., any of S. africanum Stapf, S. anatolicum Boiss, S. cereale L, S. ciliatiglume (Boiss.) Grossh, S. iranicum Kobyl, S. montanum Guss, S. segetale (Zhuk.) Roshev, S. sylvestre Host, or S. vavilovii Grossh). In some embodiments, the host cell is a Secale cereale cell. In some embodiments, the host cell is a rye hybrid cell. In some embodiments, the host cell is a barley cell (e.g., any of H. arizonicum, H. bogdanii, H. brachyantherum, H. brevisubulatum, H. bulbosum, H. comosum, H. depressum, H. intercedens, H. jubatum, H. marinum, H. murinum, H. parodii, H. pusillum, H. secalinum, H. spontaneum, or H. vulgare). In some embodiments, the host cell is a Hordeum vulgare cell. In some embodiments, the host cell is a barley hybrid (e.g., a hybrid of barley and wheat) cell. In some embodiments, the host cell is an oat cell (e.g., Avena sativa, A. abyssinica, A. byzantine, A. nuda, A. strigose, A. aemulans, A. barbata, A. brevis, A. chinensis, A. clauda, A. eriantha, A. fatua, A. longiglumis, A. maroccana, A. murphyi, A. prostrata, A. saxatilis, A. sterilis, A. strigose, A. vaviloviana, A. ventricosa, or A. volgensis). In some embodiments, the host cell is an Avena sativa cell.

Suitable eukaryotic microbes that may be used as host cells include, but are not limited to, filamentous fungi or yeast (e.g., Saccharomyces cerevisiae). Others examples include Schizosaccharomyces pombe; Kluyveromyces hosts such as, e.g., K. lactis (MW98-8C, CBS683, CBS4574), K. fragilis (ATCC 12,424), K. bulgaricus (ATCC 16,045), K. wickeramii (ATCC 24, 178), K. waltii (ATCC 56,500), K. drosophilarum (ATCC 36,906), K. thermotolerans, and K. marxianus; yarrowia (EP 402,226); Pichia pastoris; Candida; Trichoderma reesia; Neurospora crassa; Schwanniomyces such as Schwanniomyces occidentalis; and filamentous fungi such as, e.g., Neurospora, Penicillium, Tolypocladium; and Aspergillus hosts such as A. nidulans, and A. niger. Methyl otropic yeasts may also be used, e.g., yeast capable of growth on methanol, selected from the genera Hansenula, Candida, Kloeckera, Pichia, Saccharomyces, Torulopsis, and Rhodotorula.

Suitable vertebrate or mammalian cells that may be used as host cells include, but are not limited, to Chinese hamster ovary (CHO) cell lines, various Cos cell lines, HeLa cells, myeloma cell lines, transformed B-cells, or hybridomas. Other examples of vertebrate or mammalian cells that may be used as host cells include monkey kidney CV1 cells, e.g., transformed by SV40 (COS-7, ATCC CRL 1651); human embryonic kidney cells (293 cells); baby hamster kidney cells (BHK, ATCC CCL 10); Chinese hamster ovary cells/-DHFR(CHO or CHO-DP-12 line); mouse Sertoli cells; monkey kidney cells (CV1 ATCC CCL 70); African green monkey kidney cells (VERO-76, ATCC CRL-1587); human cervical carcinoma cells (HELA, ATCC CCL 2); canine kidney cells (MDCK, ATCC CCL 34); buffalo rat liver cells (BRL 3 A, ATCC CRL 1442); human lung cells (W138, ATCC CCL 75); human liver cells (Hep G2, HB 8065); mouse mammary tumor (MMT 060562, ATCC CCL51); TRI cells; MRC 5 cells; FS4 cells; and human hepatoma cells (Hep G2).

In some embodiments, the host cell may be modified to improve or regulate the expression of a modified gluten polypeptide of the disclosure. The host cell may, for example, be modified to include chaperone proteins, repressor proteins, or enhancer proteins to improve or regulate expression levels.

### Expression Methods

Any method for expressing or producing polypeptides known in the art or described herein may be used to produce modified gluten polypeptides of the disclosure.

In some embodiments, the recombinant nucleic acid molecule, e.g., a vector, can be transferred into the host cell by any suitable method known in the art, which will vary depending on the type of host cell. For example, calcium chloride transfection is commonly utilized for prokaryotic cells, whereas calcium phosphate treatment, electroporation, lipofection, biolistics or viral-based transfection may be used for other cellular hosts. (See generally Sambrook et al, Molecular Cloning: A Laboratory Manual, Cold Spring Harbor Press, 2nd ed., 1989). In one example, polybrene, protoplast fusion, liposomes, electroporation, or microinjection may be used to transform mammalian host cells. In another example, any method for introducing nucleic acids into plant cells known in the art or described herein (e.g., in the “Plant Transformation and Expression Methods” section of the present disclosure) may be used to transfer a nucleic acid molecule, e.g., a vector, into a plant host cell.

In some embodiments, the host cell may be cultured, grown or incubated under conditions such that the modified gluten polypeptide encoded by the nucleic acid or vector is expressed. Expression of the modified gluten polypeptide may be assessed using any suitable method known in the art, such as by assessing the amount of mRNA encoding the modified gluten polypeptide (e.g., using polymerase chain reaction (PCR), quantitative PCR methods, reverse transcription (RT)-PCR, RNA-sequencing, Northern blots, or microarray-based methods), or by assessing the amount of the modified gluten polypeptide (e.g., using immunoblots such as Western blots, enzyme-linked immunosorbent assays (ELISA), mass spectrometry, flow cytometry, immunoprecipitation, and immunohistochemistry).

In some embodiments, a transgenic multicellular host organism, e.g., a plant, which has been genetically manipulated may also be used to produce a modified gluten polypeptide. The organism may be, for example, a transgenic plant comprising a nucleic acid encoding a modified gluten polypeptide of the disclosure. In some embodiments, the organism is a modified plant comprising a nucleic acid encoding a modified gluten polypeptide of the disclosure produced according to the methods provided herein.

In some embodiments, a modified gluten polypeptide of the disclosure may also be produced using in vitro methods. For example, a modified gluten polypeptide of the disclosure may be produced by direct peptide synthesis using solid-phase techniques (see, e.g., Stewart et al, Solid-Phase Peptide Synthesis W.H. Freeman Co., San Francisco, Calif. (1969); Merrifield, J. Am. Chem. Soc. 85:2149-2154 (1963)). In vitro polypeptide synthesis may be performed using manual techniques or by automation. Automated polypeptide synthesis may be accomplished, for instance, using an Applied BioSystems Peptide Synthesizer (Foster City, Calif.) using the manufacturer's instructions. Various portions of the polypeptide may be chemically synthesized separately and combined using chemical or enzymatic methods to produce the desired polypeptide.

In some embodiments, the methods further comprise recovering the modified gluten polypeptide from the host cell, cell culture medium, or in vitro reaction mixture. Any suitable method known in the art may be used to recover the modified gluten polypeptide from the host cell, cell culture medium, or in vitro reaction mixture. For example, selective precipitation with substances such as ammonium sulfate, column chromatography (e.g., cation exchange chromatography, anion exchange chromatography, affinity chromatography, mixed-mode chromatography, metal ion affinity chromatography, reversed phase chromatography and the like), immunopurification methods, and/or immunoprecipitation methods may be used. In some embodiments, when the modified gluten polypeptide is expressed in one or more host cells, the methods may comprise cell lysis (e.g., using any suitable method, such as sonication). In some embodiments, the methods comprise recovering the polypeptide using affinity chromatography or immunoprecipitation with a specific antibody to the target polypeptide. In some embodiments, the modified gluten polypeptide may be expressed as a fusion polypeptide, e.g., fused to an affinity or purification tag. Examples of tags that may be used include a poly-histidine tag, glutathione S-transferase (GST), maltose E binding protein, protein A, an epitope of GST, thioredoxin, an epitope of thioredoxin, the FLAG epitope, or the HA antigen. Fusion polypeptides comprising an affinity or purification tag may be purified by standard means, such as using antibodies that bind to the tag, or using chromatography techniques such as nickel-affinity chromatography to purify poly-histidine-tagged polypeptides. In some embodiments, the modified gluten polypeptide expressed as a fusion polypeptide further includes a cleavable peptide joining the gluten polypeptide and the tag. In some embodiments, the methods provided herein further comprise digestion of the fusion polypeptide, e.g., with a suitable proteolytic enzyme, to release the modified gluten polypeptide from the tag. Examples of enzymes and their cognate peptide sequences that may be used according to the present disclosure include, without limitation, Factor Xa (which recognizes the sequence Ile-(Glu or Asp)-Gly-Arg-X; SEQ ID NO: 52), thrombin (which recognizes the sequence Leu-Val-Pro-Arg↓Gly-Ser; SEQ ID NO: 53), and enterokinase (which recognizes the sequence Asp-Asp-Asp-Asp-Lys-X; SEQ ID NO: 54). The modified gluten polypeptide may then be further purified by standard protein chemistry techniques.

In some embodiments, the methods described herein result in the production of an isolated modified gluten polypeptide. In some embodiments, the isolated modified gluten polypeptide is separated from other molecules, e.g., other polypeptides, carbohydrates, nucleic acids, lipids, small molecules, or other macromolecules present in the host cell, cell culture medium, or in vitro reaction mixture. In some embodiments, the isolated modified gluten polypeptide is free or substantially free of other molecules, e.g., other polypeptides, carbohydrates, nucleic acids, lipids, small molecules, or other macromolecules present in the host cell, cell culture medium, or in vitro reaction mixture. In some embodiments, the isolated modified gluten polypeptide comprises about 50% or less, about 40% or less, about 30% or less, about 20% or less, about 15% or less, about 10% or less, about 5% or less, about 2.5% or less, or about 1% or less of other molecules, e.g., other polypeptides, carbohydrates, nucleic acids, lipids, small molecules, or other macromolecules present in the host cell, cell culture medium, or in vitro reaction mixture.

### Methods of Producing Modified Plants, Plant Tissues, Plant Organs, Plant Parts, or Plant Cells

In some aspects, provided herein are methods for producing a modified plant, plant tissue, plant organ, plant part, or plant cell comprising a modified gluten polypeptide with a reduced inflammatory potential.

In some embodiments, the modified gluten polypeptide with a reduced inflammatory potential is a modified gluten polypeptide comprising a modified gluten polypeptide amino acid sequence generated according to the methods described herein, e.g., comprising one or more alterations in one or more ISGs. In some embodiments, the one or more alterations include: (i) a substitution of one or more amino acid residues, (ii) a deletion of one or more amino acid residues, and/or (iii) an insertion of one or more amino acid residues.

In some embodiments, the one or more alterations include a substitution of one or more amino acid residues in an ISG. In some embodiments, the substitution of one or more amino acid residues is a conservative or a non-conservative amino acid substitution. In some embodiments, any of between about 1 and about 5, between about 5 and about 10, between about 10 and about 15, between about 15 and about 20, between about 20 and about 25, between about 25 and about 30, between about 30 and about 35, between about 35 and about 40, between about 40 and about 45, between about 45 and about 50, between about 50 and about 55, between about 55 and about 60, between about 60 and about 65, or between about 65 and about 70 amino acids in an ISG are substituted. In some embodiments, at least 1, at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 21, at least 22, at least 23, at least 24, at least 25, at least 26, at least 27, at least 28, at least 29, at least 30, at least 31, at least 32, at least 33, at least 34, at least 35, at least 36, at least 37, at least 38, at least 39, at least 40, at least 41, at least 42, at least 43, at least 44, at least 45, at least 46, at least 47, at least 48, at least 49, at least 50, at least 51, at least 52, at least 53, at least 54, at least 55, at least 56, at least 57, at least 58, at least 59, at least 60, at least 61, at least 62, at least 63, at least 64, at least 65, at least 66, at least 67, at least 68, at least 69, or at least 70, or more amino acids in an ISG are substituted.

In some embodiments, the one or more alterations include a deletion of one or more amino acid residues in an ISG. In some embodiments, any of between about 1 and about 5, between about 5 and about 10, between about 10 and about 15, between about 15 and about 20, between about 20 and about 25, between about 25 and about 30, between about 30 and about 35, between about 35 and about 40, between about 40 and about 45, between about 45 and about 50, between about 50 and about 55, between about 55 and about 60, between about 60 and about 65, or between about 65 and about 70 amino acids in an ISG are deleted. In some embodiments, at least 1, at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 21, at least 22, at least 23, at least 24, at least 25, at least 26, at least 27, at least 28, at least 29, at least 30, at least 31, at least 32, at least 33, at least 34, at least 35, at least 36, at least 37, at least 38, at least 39, at least 40, at least 41, at least 42, at least 43, at least 44, at least 45, at least 46, at least 47, at least 48, at least 49, at least 50, at least 51, at least 52, at least 53, at least 54, at least 55, at least 56, at least 57, at least 58, at least 59, at least 60, at least 61, at least 62, at least 63, at least 64, at least 65, at least 66, at least 67, at least 68, at least 69, or at least 70, or more amino acids in an ISG are deleted.

In some embodiments, the one or more alterations include an insertion of one or more amino acid residues in an ISG. In some embodiments, any of between about 1 and about 5, between about 5 and about 10, between about 10 and about 15, between about 15 and about 20, between about 20 and about 25, between about 25 and about 30, between about 30 and about 35, between about 35 and about 40, between about 40 and about 45, between about 45 and about 50, between about 50 and about 55, between about 55 and about 60, between about 60 and about 65, or between about 65 and about 70 amino acids are inserted in an ISG. In some embodiments, at least 1, at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 21, at least 22, at least 23, at least 24, at least 25, at least 26, at least 27, at least 28, at least 29, at least 30, at least 31, at least 32, at least 33, at least 34, at least 35, at least 36, at least 37, at least 38, at least 39, at least 40, at least 41, at least 42, at least 43, at least 44, at least 45, at least 46, at least 47, at least 48, at least 49, at least 50, at least 51, at least 52, at least 53, at least 54, at least 55, at least 56, at least 57, at least 58, at least 59, at least 60, at least 61, at least 62, at least 63, at least 64, at least 65, at least 66, at least 67, at least 68, at least 69, or at least 70, or more amino acids are inserted in an ISG.

In some embodiments, the plant, plant tissue, plant organ, plant part, or plant cell to be modified is from a monocot or a dicot, such as crop plants, ornamental plants, and non-domesticated or wild plants. In some embodiments, the plant, plant tissue, plant organ, plant part, or plant cell to be modified is from a plant of commercial or agricultural interest, such as crop plants (especially crop plants used for human food or animal feed), wood- or pulp-producing trees, vegetable plants, fruit plants, and ornamental plants. In some embodiments, the plant, plant tissue, plant organ, plant part, or plant cell to be modified is from grain crop plants (such as wheat, oat, barley, maize, rye, triticale, rice, millet, sorghum, quinoa, amaranth, and buckwheat); forage crop plants (such as forage grasses and forage dicots including alfalfa, vetch, clover, and the like); oilseed crop plants (such as cotton, safflower, sunflower, soybean, canola, rapeseed, flax, peanuts, and oil palm); tree nuts (such as walnut, cashew, hazelnut, pecan, almond, and the like); sugarcane, coconut, date palm, olive, sugar beet, tea, and coffee; wood- or pulp-producing trees; vegetable crop plants such as legumes (for example, beans, peas, lentils, alfalfa, peanut), lettuce, asparagus, artichoke, celery, carrot, radish, the brassicas (for example, cabbages, kales, mustards, and other leafy brassicas, broccoli, cauliflower, Brussels sprouts, turnip, kohlrabi), cucurbits (for example, cucumbers, melons, summer squashes, winter squashes), alliums (for example, onions, garlic, leeks, shallots, chives), members of the Solanaceae (for example, tomatoes, eggplants, potatoes, peppers, ground cherries), and members of the Chenopodiaceae (for example, beet, chard, spinach, quinoa, amaranth); fruit crop plants such as apple, pear, citrus fruits (for example, orange, lime, lemon, grapefruit, and others), stone fruits (for example, apricot, peach, plum, nectarine), banana, pineapple, grape, kiwifruit, papaya, avocado, and berries; and ornamental plants including ornamental flowering plants, ornamental trees and shrubs, ornamental groundcovers, and ornamental grasses. In some embodiments, the plant, plant tissue, plant organ, plant part, or plant cell to be modified is from dicot plants, such as canola, cotton, potato, quinoa, amaranth, buckwheat, safflower, soybean, sugar beet, or sunflower. In some embodiments, the plant, plant tissue, plant organ, plant part, or plant cell to be modified is from monocot plants, such as wheat, oat, barley, maize, rye, triticale, rice, ornamental and forage grasses, sorghum, millet, and sugarcane. The plant, plant tissue, plant organ, plant part, or plant cell to be modified according to the methods of the disclosure may be or may be derived from a monocotyledonous or dicotyledonous plant or a plant cell system, including species from one of the following families: Acanthaceae, Alliaceae, Alstroemeriaceae, Amaryllidaceae, Apocynaceae, Arecaceae, Asteraceae, Berberidaceae, Bixaceae, Brassicaceae, Bromeliaceae, Cannabaceae, Caryophyllaceae, Cephalotaxaceae, Chenopodiaceae, Colchicaceae, Cucurbitaceae, Dioscoreaceae, Ephedraceae, Erythroxylaceae, Euphorbiaceae, Fabaceae, Lamiaceae, Linaceae, Lycopodiaceae, Malvaceae, Melanthiaceae, Musaceae, Myrtaceae, Nyssaceae, Papaveraceae, Pinaceae, Plantaginaceae, Poaceae, Rosaceae, Rubiaceae, Salicaceae, Sapindaceae, Solanaceae, Taxaceae, Theaceae, or Vitaceae. Suitable species also include members of the genera Abelmoschus, Abies, Acer, Agrostis, Allium, Alstroemeria, Ananas, Andrographis, Andropogon, Arundo, Atropa, Berberis, Beta, Bixa, Brassica, Calendula, Camellia, Camptotheca, Cannabis, Capsicum, Carthamus, Catharanthus, Cephalotaxus, Chrysanthemum, Cinchona, Citrullus, Coffea, Colchicum, Coleus, Cucumis, Cucurbita, Cynodon, Datura, Dianthus, Digitalis, Dioscorea, Elaeis, Ephedra, Erianthus, Erythroxylum, Eucalyptus, Festuca, Fragaria, Galanthus, Glycine, Gossypium, Helianthus, Hevea, Hordeum, Hyoscyamus, Jatropha, Lactuca, Linum, Lolium, Lupinus, Lycopersicon, Lycopodium, Manihot, Medicago, Mentha, Acanthus, Musa, Nicotiana, Oryza, Panicum, Papaver, Parthenium, Pennisetum, Petunia, Phalaris, Phleum, Pinus, Poa, Poinsettia, Populus, Rauwolfia, Ricinus, Rosa, Saccharum, Salix, Sanguinaria, Scopolia, Secale, Solanum, Sorghum, Spartina, Spinacea, Tanacetum, Taxus, Theobroma, Triticosecale, Triticum, Uniola, Veratrum, Vinca, Vitis, and Zea. Suitable species also include Panicum spp., Sorghum spp., Acanthus spp., Saccharum spp., Erianthus spp., Populus spp., Andropogon gerardii (big bluestem), Pennisetum purpureum (elephant grass), Phalaris arundinacea (reed canary grass), Cynodon dactylon (Bermuda grass), Festuca arundinacea (tall fescue), Spartina pectinata (prairie cord-grass), Medicago sativa (alfalfa), Arundo donax (giant reed), Secale cereale (rye), Salix spp. (willow), Eucalyptus spp. (eucalyptus), Triticosecale (triticum), bamboo, Helianthus annuus (sunflower), Carthamus tinctorius (safflower), Jatropha curcas (jatropha), Ricinus communis (castor), Elaeis guineensis (palm), Linum usitatissimum (flax), Brassica juncea, Beta vulgaris (sugar beet), Manihot esculenta (cassava), Lycopersicon esculentum (tomato), Lactuca sativa (lettuce), Musa paradisiaca (banana), Solanum tuberosum (potato), Brassica oleracea (broccoli, cauliflower, Brussels sprouts), Camellia sinensis (tea), Fragaria ananassa (strawberry), Theobroma cacao (cocoa), Coffea arabica (coffee), Vitis vinfera (grape), Ananas comosus (pineapple), Capsicum annum (hot & sweet pepper), Allium cepa (onion), Cucumis melo (melon), Cucumis sativus (cucumber), Cucurbita maxima (squash), Cucurbita moschata (squash), Spinacea oleracea (spinach), Citrullus lanatus (watermelon), Abelmoschus esculentus (okra), Solanum melongena (eggplant), Rosa spp. (rose), Dianthus caryophyllus (carnation), Petunia spp. (petunia), Poinsettia pulcherrima (poinsettia), Lupinus albus (lupin), Uniola paniculata (oats), bentgrass (Agrostis spp.), Populus tremuloides (aspen), Pinus spp. (pine), Abies spp. (fir), Acer spp. (maple), Hordeum vulgare (barley), Poa pratensis (bluegrass), Lolium spp. (ryegrass) and Phleum pratense (timothy), Panicum virgatum (switch grass), Sorghum bicolor (sorghum, sudan grass), Miscanthus giganteus, Saccharum sp. (energy cane), Populus balsamifera (poplar), Zea mays (corn), Glycine max (soybean), Brassica napus (canola), Triticum aestivum (wheat), Gossypium hirsutum (cotton), Oryza sativa (rice), Helianthus annuus (sunflower), Medicago sativa (alfalfa), Beta vulgaris (sugar beet), or Pennisetum glaucum (pearl millet).

In some embodiments, the plant, plant tissue, plant organ, plant part, or plant cell to be modified is a wheat (e.g., any of T. aestivum, T aethiopicum, T araraticum, T. boeoticum, T carthlicum, T compactum, T. dicoccoides, T. dicoccon, T. durum, T. ispahanicum, T. karamyschevii, T. macha, T. militinae, T. monococcum, T. polonicum, T. spelta, T. sphaerococcum, T. timopheevii, T. turanicum, T. turgidum, T urartu, T. vavilovii, or T. zhukovskyi) plant, plant tissue, plant organ, plant part, or plant cell. In some embodiments, the plant, plant tissue, plant organ, plant part, or plant cell to be modified is a T. aestivum plant, plant tissue, plant organ, plant part, or plant cell. In some embodiments, the plant, plant tissue, plant organ, plant part, or plant cell to be modified is a wheat hybrid plant, plant tissue, plant organ, plant part, or plant cell. In some embodiments, the plant, plant tissue, plant organ, plant part, or plant cell to be modified is a wheat and rye hybrid (e.g., triticale) plant, plant tissue, plant organ, plant part, or plant cell.

In some embodiments, the plant, plant tissue, plant organ, plant part, or plant cell to be modified is a rye (e.g., any of S. africanum Stapf, S. anatolicum Boiss, S. cereale L, S. ciliatiglume (Boiss.) Grossh, S. iranicum Kobyl, S. montanum Guss, S. segetale (Zhuk.) Roshev, S. sylvestre Host, or S. vavilovii Grossh) plant, plant tissue, plant organ, plant part, or plant cell. In some embodiments, the plant, plant tissue, plant organ, plant part, or plant cell to be modified is a Secale cereale plant, plant tissue, plant organ, plant part, or plant cell. In some embodiments, the plant, plant tissue, plant organ, plant part, or plant cell to be modified is a rye hybrid (e.g., a wheat and rye hybrid, such as triticale) plant, plant tissue, plant organ, plant part, or plant cell.

In some embodiments, the plant, plant tissue, plant organ, plant part, or plant cell to be modified is a barley (e.g., any of H. arizonicum, H. bogdanii, H. brachyantherum, H. brevisubulatum, H. bulbosum, H. comosum, H. depressum, H. intercedens, H. jubatum, H. marinum, H. murinum, H. parodii, H. pusillum, H. secalinum, H. spontaneum, or H. vulgare) plant, plant tissue, plant organ, plant part, or plant cell. In some embodiments, the plant, plant tissue, plant organ, plant part, or plant cell to be modified is a Hordeum vulgare plant, plant tissue, plant organ, plant part, or plant cell. In some embodiments, the plant, plant tissue, plant organ, plant part, or plant cell to be modified is a barley hybrid (e.g., a hybrid of barley and wheat) plant, plant tissue, plant organ, plant part, or plant cell.

In some embodiments, the plant, plant tissue, plant organ, plant part, or plant cell to be modified is an oat (e.g., any of Avena sativa, A. abyssinica, A. byzantine, A. nuda, A. strigose, A. aemulans, A. barbata, A. brevis, A. chinensis, A. clauda, A. eriantha, A. fatua, A. longiglumis, A. maroccana, A. murphyi, A. prostrata, A. saxatilis, A. sterilis, A. strigose, A. vaviloviana, A. ventricosa, or A. volgensis) plant, plant tissue, plant organ, plant part, or plant cell. In some embodiments, the plant, plant tissue, plant organ, plant part, or plant cell to be modified is an Avena sativa plant, plant tissue, plant organ, plant part, or plant cell. In some embodiments, the plant, plant tissue, plant organ, plant part, or plant cell to be modified is an oat hybrid plant, plant tissue, plant organ, plant part, or plant cell.

In some embodiments, the plant, plant tissue, plant organ, plant part, or plant cell to be modified may be or may be derived from a naturally occurring, a mutant, a non-naturally occurring, or a transgenic plant, plant tissue, plant organ, plant part, or plant cell of any family, genus, species, variety, or cultivar described herein.

In some embodiments, the plant to be modified is an immature whole plant or a mature whole plant, or a plant from which seed or grain or anthers have been removed. In some embodiments, the plant to be modified is a seed or a plant embryo.

Examples of plant tissues to be modified include, without limitation, dermal tissue, ground tissue, or vascular tissue. Examples of plant organs to be modified include, without limitation, organs of the shoot system (e.g., leaves, buds, stems, flowers, or fruits), organs of the root system (e.g., roots, tubers, or rhizomes). Examples of plant parts to be modified include, without limitation, protoplasts, leaves, stems, roots, root tips, anthers, pistils, seed, embryo, pollen, ovules, cotyledon, hypocotyl, pod, flower, shoot, tissue, petiole, cells, meristematic cells, and the like. Examples of plant cells to be modified include, without limitation, parenchyma cells, collenchyma cells, cells of the sclerenchyma (e.g., sclereids, fibres), cells of the xylem (e.g., tracheids or vessel elements), cells of the phloem (e.g., sieve tubes, companion cells, parenchyma cells, phloem fibres or sclereids), or cells of the epidermis (e.g., stomatal guard cells). In some embodiments, the plant cell to be modified is isolated, e.g., in tissue culture, or is incorporated in a plant or plant part.

### Plant Transformation and Expression Methods

In some embodiments, the methods for producing a modified plant, plant tissue, plant organ, plant part, or plant cell comprising a modified gluten polypeptide with a reduced inflammatory potential include introducing a nucleic acid molecule encoding the modified gluten polypeptide into the plant, plant tissue, plant organ, plant part, or plant cell.

In some embodiments, a plant, plant tissue, plant organ, plant part, or plant cell of the disclosure is modified to express one or more modified gluten polypeptides with a reduced inflammatory potential. In some embodiments, the one or more modified gluten polypeptides with a reduced inflammatory potential comprise an amino acid sequence generated according to the methods provided herein. In some embodiments, the one or more modified gluten polypeptides with a reduced inflammatory potential are produced according to the methods provided herein. In some embodiments, the one or more modified gluten polypeptides with a reduced inflammatory potential comprise one or more alterations in an ISG described herein.

### Vectors

In some embodiments, a recombinant nucleic acid molecule comprising a nucleotide sequence encoding a modified gluten polypeptide may be introduced into a plant, plant tissue, plant organ, plant part, or plant cell. One of skill in the art can readily arrive at one or more nucleotide sequences encoding a modified gluten polypeptide of the disclosure. In some embodiments, a nucleotide sequence encoding a modified gluten polypeptide of the disclosure is optimized to improve expression of the gluten polypeptide in a plant, for example to account for the specific codon preferences of such plant. See, e.g., Murray et al., Nucl. Acids Res. (1989) 17: 477-498.

In some embodiments, the recombinant nucleic acid molecule is a vector, e.g., an expression vector, comprising a nucleic acid sequence encoding a modified gluten polypeptide of the disclosure. An expression vector will typically contain a nucleic acid encoding a recombinant polypeptide (e.g., a gluten polypeptide with a reduced inflammatory potential), operably linked to transcriptional initiation regulatory sequences which will direct the transcription of the nucleic acid in the intended host, e.g., in a plant, plant tissue, plant organ, plant part, or plant cell.

Typical vectors useful for expression of recombinant nucleic acids in higher plants are well known in the art and include, for example, vectors derived from the tumor-inducing (Ti) plasmid of Agrobacterium tumefaciens (e.g., see Rogers et al., Meth. in Enzymol. (1987) 153:253-277). These vectors are plant integrating vectors that integrate a portion of vector DNA into the genome of the host plant. Exemplary A. tumefaciens vectors useful herein are plasmids pKYLX6 and pKYLX7 (e.g., see of Schardl et al., Gene (1987) 61:1-11; and Berger et al., Proc. Natl. Acad. Sci. USA (1989) 86:8402-8406); and plasmid pBI 101.2 that is available from Clontech Laboratories, Inc. (Palo Alto, CA).

In some embodiments, the vector includes one or more additional elements, such as a promoter regulatory region (e.g., one conferring inducible, constitutive, environmentally-regulated, developmentally-regulated, cell-specific/selective, or tissue-specific/selective expression), a transcription initiation start site, a ribosome binding site, an RNA processing signal, a transcription termination site, and/or a polyadenylation signal.

A plant promoter, or functional fragment thereof, can be employed to control the expression of a recombinant nucleic acid of the present disclosure, e.g., in a plant, plant tissue, plant organ, plant part, or plant cell. The selection of the promoter used in expression vectors will determine the spatial and temporal expression pattern of the recombinant nucleic acid in the modified plant, plant tissue, plant organ, plant part, or plant cell, e.g., such that the nucleic acid encoding the modified gluten polypeptide of the present disclosure is only expressed in the desired tissue or at a certain time in plant development or growth. Certain promoters will express recombinant nucleic acids in all plant tissues and are active under most environmental conditions and states of development or cell differentiation (i.e., constitutive promoters). Other promoters will express recombinant nucleic acids in specific cell types (such as leaf epidermal cells, mesophyll cells, root cortex cells) or in specific tissues or organs (roots, leaves or flowers, for example). Alternatively, the selected promoter may drive expression of the recombinant nucleic acid under various inducing conditions.

Examples of suitable constitutive promoters may include, for example, the core promoter of the Rsyn7, the core CaMV 35S promoter (Odell et al., Nature (1985) 313:810-812), CaMV 19S promoter (Lawton et al., 1987), rice actin promoter (Wang et al., 1992; U.S. Pat. No. 5,641,876; and McElroy et al., Plant Cell (1985) 2:163-171), ubiquitin promoter (Christensen et al., Plant Mol. Biol. (1989)12:619-632; and Christensen et al., Plant Mol. Biol. (1992) 18:675-689), pEMU promoter (Last et al., Theor. Appl. Genet. (1991) 81:581-588), MAS promoter (Velten et al., EMBO J. (1984) 3:2723-2730), nos promoter (Ebert et al., 1987), Adh promoter (Walker et al., 1987), the P- or 2′-promoter derived from T-DNA of Agrobacterium tumefaciens, the Smas promoter, the cinnamyl alcohol dehydrogenase promoter (U.S. Pat. No. 5,683,439), rubisco promoter, GRP 1-8 promoter, and other transcription initiation regions from various plant genes known to skilled artisans. Additional examples of constitutive promoters are described in, for example, U.S. Pat. Nos. 5,608,149; 5,608,144; 5,604,121; 5,569,597; 5,466,785; 5,399,680; 5,268,463; and 5,608,142.

Examples of suitable tissue-specific promoters may include, for example, the lectin promoter (Vodkin et al., 1983; Lindstrom et al., 1990), the corn alcohol dehydrogenase 1 promoter (Vogel et al., 1989; Dennis et al., 1984), the corn light harvesting complex promoter (Simpson, 1986; Bansal et al., 1992), the corn heat shock protein promoter (Odell et al., Nature (1985) 313:810-812; Rochester et al., 1986), the pea small subunit RuBP carboxylase promoter (Poulsen et al., 1986; Cashmore et al., 1983), the Ti plasmid mannopine synthase promoter (Langridge et al., 1989), the Ti plasmid nopaline synthase promoter (Langridge et al., 1989), the petunia chalcone isomerase promoter (Van Tunen et al., 1988), the bean glycine rich protein 1 promoter (Keller et al., 1989), the truncated CaMV 35s promoter (Odell et al., Nature (1985) 313:810-812), the potato patatin promoter (Wenzler et al., 1989), the root cell promoter (Conkling et al., 1990), the maize zein promoter (Reina et al., 1990; Kriz et al., 1987; Wandelt and Feix, 1989; Langridge and Feix, 1983; Reina et al., 1990), the globulin-1 promoter (Belanger and Kriz et al., 1991), the α-tubulin promoter, the cab promoter (Sullivan et al., 1989), the PEPCase promoter (Hudspeth & Grula, 1989), the R gene complex-associated promoters (Chandler et al., 1989), and the chalcone synthase promoters (Franken et al., 1991).

Alternatively, the plant promoter can direct expression of a recombinant nucleic acid of the present disclosure in a specific tissue or may be otherwise under more precise environmental or developmental control. Such promoters are referred to herein as “inducible” promoters. Environmental conditions that may affect transcription by inducible promoters include, for example, pathogen attack, anaerobic conditions, or the presence of light. Examples of inducible promoters include, for example, the AdhI promoter which is inducible by hypoxia or cold stress, the Hsp70 promoter which is inducible by heat stress, and the PPDK promoter which is inducible by light. Examples of promoters under developmental control include, for example, promoters that initiate transcription only, or preferentially, in certain tissues, such as leaves, roots, fruit, seeds, or flowers. An exemplary promoter is the anther specific promoter 5126 (U.S. Pat. Nos. 5,689,049 and 5,689,051). The operation of a promoter may also vary depending on its location in the genome. Thus, an inducible promoter may become fully or partially constitutive in certain locations.

Moreover, any combination of a constitutive or inducible promoter, and a non-tissue-specific or tissue-specific promoter may be used to control the expression of a modified gluten polypeptide of the present disclosure.

Both heterologous and endogenous promoters can be employed to direct expression of recombinant nucleic acids of the present disclosure. Accordingly, in certain embodiments, expression of a nucleic acid encoding a modified gluten polypeptide of the present disclosure is under the control of its respective endogenous promoter. In other embodiments, expression of a nucleic acid encoding a modified gluten polypeptide of the present disclosure is under the control of a heterologous promoter.

A vector of the disclosure may also contain a regulatory sequence that serves as a 3′ terminator sequence. One of skill in the art would readily recognize a variety of terminators that may be used in the recombinant nucleic acids of the present disclosure. For example, a recombinant nucleic acid of the present disclosure may contain a 3′ NOS terminator. Further, a native terminator from an endogenous gene corresponding to a modified gluten polypeptide of the disclosure may also be used in the recombinant nucleic acids of the present disclosure.

In addition to regulatory domains, a vector of the disclosure may include additional elements, for example to express a modified gluten polypeptide of the disclosure as a fusion polypeptide. For example, a gluten polypeptide of the disclosure may be coupled to a maltose binding protein (“MBP”), glutathione S transferase (GST), hexahistidine, c-myc, or the FLAG epitope for ease of purification, monitoring expression, or monitoring cellular and subcellular localization.

In some embodiments, vectors of the disclosure may be modified to improve expression of the modified gluten polypeptide by using codon preference. When the vector is prepared or altered synthetically, advantage can be taken of known codon preferences of the intended host where the nucleic acid is to be expressed. For example, vectors of the present disclosure can be modified to account for the specific codon preferences and GC content preferences of monocotyledons and dicotyledons, as these preferences have been shown to differ (Murray et al., Nucl. Acids Res. (1989) 17: 477-498).

In some embodiments, a vector of the disclosure may include elements that target the encoded modified gluten polypeptide to a specific organelle within a plant cell. Targeting can be achieved by providing the recombinant polypeptide with an appropriate targeting peptide sequence. Examples of such targeting peptides include, for example, secretory signal peptides (for secretion, cell wall, membrane targeting), plastid transit peptides, chloroplast transit peptides, mitochondrial target peptides, vacuole targeting peptides, nuclear targeting peptides, and the like (e.g., see Reiss et al., Mol. Gen. Genet. (1987) 209(1):116-121; Settles and Martienssen, Trends Cell Biol (1998) 12:494-501; Scott et al., J Biol Chem (2000) 10:1074; and Luque and Correas, J Cell Sci (2000) 113:2485-2495).

In some embodiments, a vector of the disclosure may further include a nucleic acid sequence encoding a suppressor of gene silencing, e.g., derived from a virus. Examples of suppressors of gene silencing that may be used include, but are not limited to, the p1 protein of rice yellow mottle virus (RYMV), the p25 protein of potato virus X (PVX), the AC2 protein of African cassava mosaic virus (ACMV), the 2b protein of cucumber mosaic virus (CMV), the 19 kDa p19 protein of Cucumber necrosis virus (CNV), the helper-component proteinase (HcPro) of potato virus Y (PVY), tobacco etch virus (TEV) and Tomato bushy stunt virus (TBSV), the HcPro of tobacco etch virus (TEV), or the p19 protein of Tomato bushy stunt virus (TBSV).

In some embodiments, an endogenous gene corresponding to a modified gluten polypeptide of the present disclosure can be modified, e.g., using a gene knock out and/or a gene knock-in approach, so that the modified gluten polypeptide will be under the control of its respective endogenous elements. In some embodiments, a modified form of an entire gene corresponding to a modified gluten polypeptide of the disclosure and its surrounding genomic sequences (e.g., regulatory sequences such as promoters, enhancers, polyadenylation sequences, etc.) may be introduced into a plant, plant tissue, plant organ, plant part, or plant cell, so that the modified gluten polypeptide will be under the control of its endogenous elements, and the corresponding endogenous wild-type gene remains intact.

Any or all of these techniques may also be combined to direct the expression of a recombinant nucleic acid of the present disclosure.

### Transformation and Expression of Gluten Polypeptides

Any method known in the art for introducing nucleic acids into plants, plant tissues, plant organs, plant parts, or plant cells may be used in the methods of the disclosure. As is known in the art, protocols for transforming plants, plant tissues, plant organs, plant parts, or plant cells with recombinant nucleic acids (e.g., vectors) may vary depending on the type of plant, plant tissue, plant organ, plant part, or plant cell targeted for transformation. Suitable methods include, for example, microinjection (Crossway et al., Biotechniques (1986) 4:320-334), electroporation (Riggs et al., Proc. Natl. Acad Sci. USA (1986) 83:5602-5606), Agrobacterium-mediated transformation (e.g., using A. radiobacter, A. rhizogenes, A. rubi, or A. tumefaciens; see, e.g., U.S. Pat. No. 5,563,055), electroporation and infiltration by Agrobacterium cells (also referred to as agroinfiltration), direct gene transfer (Paszkowski et al., EMBO J. (1984) 3:2717-2722), biolistics methods, gene gun techniques, and ballistic particle acceleration (U.S. Pat. No. 4,945,050; Tomes et al. (1995) “Direct DNA Transfer into Intact Plant Cells via Microprojectile Bombardment,” in Plant Cell, Tissue, and Organ Culture: Fundamental Methods, ed. Gamborg and Phillips (Springer-Verlag, Berlin); and McCabe et al., Biotechnology (1988) 6:923-926).

In some embodiments, a modified gluten polypeptide of the present disclosure may be transiently expressed in a plant, plant tissue, plant organ, plant part, or plant cell via viral infection, or by introducing a modified gluten polypeptide-encoding RNA into the plant, plant tissue, plant organ, plant part, or plant cell to temporarily express the modified gluten polypeptide. Methods of introducing recombinant polypeptides via viral infection or via the introduction of nucleic acids such as DNAs or RNAs into plants, plant tissues, plant organs, plant parts, or plant cells are well known in the art. For example, Tobacco rattle virus (TRV) has been successfully used to introduce zinc finger nucleases in plants to cause genome modification (“Nontransgenic Genome Modification in Plant Cells”, Plant Physiology 154:1079-1087 (2010)).

In some embodiments, introduction of a nucleic acid or vector of the disclosure into a plant, plant tissue, plant organ, plant part, or plant cell results in stable expression of the modified gluten polypeptide encoded by the nucleic acid or vector. Typically, stable expression will result in the integration of the nucleic acid or vector into the host genome so as to create a transgenic plant, and the nucleic acid will be passed onto the next generation.

In other embodiments, the introduction of a nucleic acid or vector into a plant, plant tissue, plant organ, plant part, or plant cell may give rise to transient expression of the modified gluten polypeptide encoded by the nucleic acid or vector. Transient expression does not necessarily rely on the integration of the nucleic acid into the host genome. In some embodiments, introduction of a nucleic acid or vector into a plant, plant tissue, plant organ, plant part, or plant cell results in expression of the modified gluten polypeptide encoded by the nucleic acid or vector for at least about 1 day, at least about 5 days, at least about 10 days, at least about 15 days, at least about 20 days, or more after introduction of the nucleic acid or vector into the plant, plant tissue, plant organ, plant part, or plant cell.

In some embodiments, the plant, plant tissue, plant organ, plant part, or plant cell into which the nucleic acids or vectors have been introduced can be cultured, grown or incubated under conditions such that nucleic acid or vector is retained, e.g., using a selectable marker. In some embodiments, the plant, plant tissue, plant organ, plant part, or plant cell into which the nucleic acids or vectors have been introduced can be cultured, grown or incubated under conditions such that the modified gluten polypeptide encoded by the nucleic acid or vector is expressed. Expression of the modified gluten polypeptide may be assessed using any suitable method known in the art, such as by assessing the amount of mRNA encoding the modified gluten polypeptide (e.g., using polymerase chain reaction (PCR), quantitative PCR methods, reverse transcription (RT)-PCR, RNA-sequencing, Northern blots, or microarray-based methods), or by assessing the amount of the modified gluten polypeptide (e.g., using immunoblots such as Western blots, enzyme-linked immunosorbent assays (ELISA), mass spectrometry, flow cytometry, immunoprecipitation, and immunohistochemistry).

In some embodiments, progeny obtained from the plant, plant tissue, plant organ, plant part, or plant cell into which the nucleic acids or vectors have been introduced are obtained, and such progeny may be used to prepare transgenic seeds, or alternatively, bred with another plant. The seeds obtained from such progeny may be germinated, cultivated, and used to prepare subsequent generations of offspring which comprise the nucleic acid or vector originally introduced. An immature embryo or embryogenic calli from a plant may be used as a starting material. These techniques are routine and well known to one of ordinary skill in the art.

In certain embodiments, the plants, plant tissues, plant organs, plant parts, or plant cells are provided the modified gluten polypeptide through exogenous delivery of the polypeptide directly to the plants, plant tissues, plant organs, plant parts, or plant cells without the need to express a recombinant nucleic acid encoding the recombinant polypeptide in the plants, plant tissues, plant organs, plant parts, or plant cells.

In some embodiments, the methods provided herein further comprise maintaining the modified plants, plant tissues, plant organs, plant parts, or plant cells comprising a modified gluten polypeptide of the disclosure under conditions such that the modified gluten polypeptide is expressed.

### Modification of Endogenous Gluten Polypeptides

In some embodiments, the methods described herein comprise modifying an endogenous gluten polypeptide (or an endogenous gene encoding a gluten polypeptide) in the plant, plant tissue, plant organ, plant part, or plant cell.

In some embodiments, the methods comprise reducing the expression of one or more endogenous gluten polypeptides in the plant, plant tissue, plant organ, plant part, or plant cell. Any suitable method for modulating gene expression in plants known in the art may be used. In some embodiments, reducing the expression of one or more endogenous gluten polypeptides in the plant, plant tissue, plant organ, plant part, or plant cell is performed using gene silencing methods. As is known in the art, a suppression construct targeting the one or more endogenous gluten polypeptides may be transformed or stably integrated into the genome of the plant, plant tissue, plant organ, plant part, or plant cell. Suppression constructs are well-known in the art, and are readily constructed once the target gene of interest is selected, and include, without limitation, co-suppression constructs, antisense constructs, viral-suppression constructs, hairpin suppression constructs, stem-loop suppression constructs, double-stranded RNA-producing constructs, and more generally, RNAi (RNA interference) constructs, and small RNA constructs such as siRNA (short interfering RNA) constructs and miRNA (microRNA) constructs. See, e.g., Pandey et al., Advances in Plant Gene Silencing Methods, In: Mysore K., Senthil-Kumar M. (eds) Plant Gene Silencing. Methods in Molecular Biology, 2015, vol 1287, for a review of methods of gene silencing in plants. In some embodiments, the suppression construct comprises a nucleotide sequence derived from a target gene of interest, such as a gene encoding the one or more gluten polypeptides, and may comprise all or part of the nucleic acid sequence of the sense strand (or antisense strand) of the target gene of interest. Depending upon the approach to be utilized, the region may be 100% identical or less than 100% identical (e.g., at least 50% or any integer between 51% and 100% identical) to all or part of the sense strand (or antisense strand) of the gene of interest. In some embodiments, such suppression constructs, upon introduction into a plant cell, result in silencing of the target gene. In some embodiments, silencing of the target gene comprises a reduction of the mRNA levels or protein levels of the targeted gene. For additional information about methods for reducing expression of genes in plants, which may be used according to the methods provided herein, see, e.g., U.S. Pat. No. 5,107,065 describing anti-sense RNA-based methods; Vaucheret, et al., (1998) Plant J. 16:651-659 and Gura, (2000) Nature 404:804-808, describing co-suppression methods; PCT Publication WO 1998/36083, describing viral sequence-based methods; Wesley, et al., (2003) Methods in Molecular Biology, Plant Functional Genomics: Methods and Protocols 236:273-286, PCT Publication WO 1999/53050, PCT Publication WO 1999/61632, PCT Publication WO 2002/00894, and PCT Publication WO 2002/00904, describing methods using sequences comprising hairpins and stem-loops; and Saurabh et al., Planta (2014) 239:543-564 for a review of RNAi-based methods.

In some embodiments, the methods provided herein comprise introducing one or more alterations in one or more genes encoding endogenous gluten polypeptides in the plant, plant tissue, plant organ, plant part, or plant cell. In some embodiments, the one or more alterations result in (i) a substitution of one or more amino acid residues, (ii) a deletion of one or more amino acid residues, and/or (iii) an insertion of one or more amino acid residues in the encoded endogenous gluten polypeptide. In some embodiments, the one or more alterations comprise a deletion of all or a portion of one or more genes encoding endogenous gluten polypeptides in the plant, plant tissue, plant organ, plant part, or plant cell. In some embodiments, the one or more alterations result in reduced expression, stability, or half-life of the encoded endogenous gluten polypeptide. In some cases, it may desirable to produce a modified plant, plant tissue, plant organ, plant part, or plant cell that comprises a gluten polypeptide with a reduced inflammatory potential, wherein one or more alterations in one or more ISGs, e.g., as described in detail herein, are introduced in the endogenous genes encoding the gluten polypeptides. In some cases, it may desirable to produce a modified plant, plant tissue, plant organ, plant part, or plant cell that comprises a gluten polypeptide with a reduced inflammatory potential, wherein one or more genes encoding endogenous gluten polypeptides have been replaced with one or more nucleotide sequences encoding a modified gluten polypeptide with a reduced inflammatory potential of the disclosure.

Any suitable methods for specific gene targeting and genome editing in plants may be used in the methods described herein.

In some embodiments, sequence-specific nucleases may be used to modify one or more genes encoding endogenous gluten polypeptides in the plant, plant tissue, plant organ, plant part, or plant cell, e.g., zinc finger nucleases (ZFNs) and transcription activator-like effector nucleases (TALENs). As will be recognized by one of skill in the art, ZFNs and TALENs comprise a programmable DNA binding domain and a nuclease (e.g., the FokI nuclease). Upon introduction and expression of ZFN or TALEN constructs in cells, the DNA binding domain specifically binds to a corresponding DNA sequence and guides the nuclease to make a specific DNA strand cleavage. A pair of ZFNs or TALENs can be introduced to generate double strand breaks (DSBs), which activate the DNA repair systems and significantly increase the frequency of both non-homologous end joining (NHEJ) and homologous recombination (HR). In general, a single zinc-finger motif specifically recognizes 3 bp, and engineered zinc-finger with tandem repeats can recognize up to 9-36 bp. ZFNs have been used in plants to introduce small mutations, gene deletions, or for foreign DNA integration (gene replacement/knock-in) at the specific genomic sites. In contrast with the zinc finger protein, TALENs are derived from the plant pathogenic bacteria Xanthomonas and contain 34 amino acid tandem repeats in which repeat-variable diresidues (RVDs) at positions 12 and 13 determine the DNA-binding specificity. TALENs can specifically recognize genomic sequences and the coupled chimeric nuclease can generate DSBs at specific genomic sites. TALEN-mediated genome editing has been used in many organisms including yeast, animals, and plants.

In some embodiments, clustered regularly interspaced short palindromic repeats (CRISPR)-based methods may be used to modify one or more genes encoding endogenous gluten polypeptides in the plant, plant tissue, plant organ, plant part, or plant cell, e.g., using CRISPR-Cas9-based methods. As will be recognized by one of skill in the art, CRISPR and the associated nucleases, such as Cas9, are part of adaptive immunity in bacteria and archaea. The Cas9 endonuclease, a component of Streptococcuspyogenes type II CRISPR/Cas system, forms a complex with two short RNA molecules called CRISPR RNA (crRNA) and transactivating crRNA (transcrRNA), which guide the nuclease to cleave non-self DNA on both strands at a specific site. The crRNA-transcrRNA heteroduplex can be replaced by one chimeric RNA, termed a guide RNA (gRNA), which can then be programmed to target specific sites, e.g., one or more genes encoding endogenous gluten polypeptides. The targeting specificity is determined by an approximately 20-nucleotide sequence at the 5′ end of the gRNA. The desired target sequence precedes a protospacer adjacent motif (PAM) which is a short DNA sequence usually 2-6 base pairs in length that follows the DNA region targeted for cleavage by the CRISPR system, such as CRISPR-Cas9. The PAM sequence is required for the Cas nuclease to cleave DNA, and is generally found 3-4 nucleotides downstream from the cut site. After base pairing of the gRNA to the target, Cas9 mediates a double strand break about 3-nucleotides upstream of PAM. Generally, the GC content of a gRNA sequence is between about 40-80%. The CRISPR/Cas system has been demonstrated for genome editing in human, mice, zebrafish, yeast, plants, and bacteria. In plants, nucleic acids such as vectors encoding the components of the CRISPR/Cas system (e.g., the gRNA and the Cas nuclease) may be delivered using any methods for introducing nucleic acids into plant cells known in the art or provided herein. For example, Agrobacterium-mediated transformation, biolistic bombardment, or protoplast transformation may be used. In some cases, the Cas nuclease and the gRNA can be combined in vitro before being delivered into cells as ribonucleoprotein (RNP) complexes.

For a review of methods for specific gene targeting and genome editing in plants that may be used in any of the methods provided herein, see, e.g., Razzaq et al., Int J Mol Sci (2019) 20(16):4045.

### Reduced Inflammatory Potential

In some embodiments, a modified gluten polypeptide of the disclosure, such as a modified gluten polypeptide produced according to the methods provided herein, or a modified gluten polypeptide present in or obtained from a modified plant, plant tissue, plant organ, plant part, or plant cell of the disclosure, has a reduced inflammatory potential, e.g., when ingested by an individual such as a human, e.g., as compared to an unmodified gluten polypeptide. In some embodiments, the modified gluten polypeptide of the disclosure has a reduced ability to remodel a cell membrane, e.g., when ingested by an individual such as a human, e.g., as compared to an unmodified gluten polypeptide. In some embodiments, the modified gluten polypeptide of the disclosure has a reduced ability to access an endosomal compartment within a cell, e.g., when ingested by an individual such as a human, e.g., as compared to an unmodified gluten polypeptide. In some embodiments, the modified gluten polypeptide of the disclosure has a reduced ability to mediate organization of innate immune ligands (e.g., dsDNA, dsRNA, ssDNA, or ssRNA) into ordered nanocrystalline structures, e.g., when ingested by an individual such as a human, e.g., as compared to an unmodified gluten polypeptide. In some embodiments, the modified gluten polypeptide of the disclosure has a reduced ability to promote activation of Toll-Like Receptors (TLRs), e.g., when ingested by an individual such as a human, e.g., as compared to an unmodified gluten polypeptide. In some embodiments, the modified gluten polypeptide of the disclosure has a reduced ability to self-assemble into amyloidal or protofibril structures to activate formyl peptide receptor-like 1 (FPRL1) and/or formyl peptide receptor 2 (FPR2), e.g., when ingested by an individual such as a human, e.g., as compared to an unmodified gluten polypeptide.

### Gluten-Related Disorders

Gluten-related disorders include, without limitation, celiac disease, including the various subtypes, e.g., classical celiac disease, atypical celiac disease, latent celiac disease, and silent celiac disease, dermatitis herptiformis, gluten ataxia, gluten allergy, gluten intolerance, gluten toxicity, and gluten sensitivity. Symptoms of gluten-related disorders, e.g., that occur upon ingestion of gluten or gluten-related polypeptides, include, without limitation, inflammation (e.g., in the gut), abdominal pain, eczema, rashes, headaches, “foggy mind,” autoimmune reactions, gastrointestinal symptoms such as diarrhea, steatorrhea, abdominal distension, weight loss, anemia, osteoporosis, arthritis, infertility, peripheral neuropathy, liver failure, and depression.

In some embodiments, a modified gluten polypeptide of the disclosure, such as a modified gluten polypeptide produced according to the methods provided herein, or a modified gluten polypeptide present in or obtained from a modified plant, plant tissue, plant organ, plant part, or plant cell of the disclosure, has a reduced ability to elicit one or more symptoms of a gluten-related disorder in an individual, e.g., upon ingestion of the modified gluten polypeptide by the individual, e.g., as compared to an unmodified gluten polypeptide. In some embodiments, ingestion by an individual of the modified gluten polypeptide of the disclosure results in a reduction in the severity, frequency, and/or seriousness of one or more symptoms of a gluten-related disorder by at least about 5%, at least about 10%, at least about 20%, at least about 30%, at least about 40%, at least about 50%, at least about 60%, at least about 70%, at least about 80%, at least about 90%, at least about 95%, at least about 99%, or 100%, e.g., as compared to the severity, frequency, and/or seriousness of the one or more symptoms upon ingestion of an unmodified gluten polypeptide.

### Celiac Disease

Celiac disease, also known as celiac spruce or gluten-sensitive enteropathy, is an immune disease characterized by an immune reaction to ingesting gluten. Diagnosis of celiac disease typically relies on multiple criteria including: 1) presentation with typical celiac disease symptoms; 2) positivity of serological tests, including, for example, high titer IgA antibodies to tTG (anti-tTG), high titer antibodies to deamidated α-gliadin peptides; 3) HLA-DQ2 and/or HLA-DQ8 genotypes; 4) celiac enteropathy found on small bowel biopsy; and 5) response to a gluten-free diet. In some embodiments, diagnosis of celiac disease is confirmed if at least four of the five criteria are fulfilled.

Celiac disease prevalence is increased in at-risk conditions, such as a family history of celiac disease, autoimmune diseases, IgA deficiency, some genetic syndromes (Down syndrome, Turner syndrome and William syndromes) and especially type 1 diabetes and thyroiditis. Celiac disease is strongly associated with specific human leukocyte antigen (HLA) class II genes, HLA-DQ2 and HLA-DQ8, located on chromosome 6p21. Most celiac disease patients (approximately 95%) express genes encoding the major histocompatibility complex (MHC) class II protein HLA-DQ2. The remaining patients are usually HLA-DQ8-positive. The HLA-DQ2 haplotype is common and is carried by approximately 30% of Caucasian individuals, implying that the presence of HLA-DQ2 and/or HLA-DQ8 is necessary for disease development but not sufficient on its own as its estimated risk effect is only 36% to 53%. Non-HLA genes also contribute to celiac disease predisposition.

Presentation of celiac disease can vary widely. Celiac disease typically presents in children as a disease of failure to thrive associated with symptoms of malabsorption, e.g., weight loss, steatorrhea, and multiple deficiencies, although other extra-intestinal symptoms, for example, failure of axial height development and delayed menarche in girls may be present.

Several subtypes of celiac disease have been described, including classical celiac disease, atypical celiac disease, latent celiac disease, and silent celiac disease. Regardless of the subtype, many celiac disease cases go undiagnosed, which exposes patients to the risk of long-term complications, for example, infertility and malignancies, e.g., lymphoma and intestinal carcinoma.

Symptoms associated with classical celiac disease include, without limitation, diarrhea, abdominal distension, and failure to thrive. These symptoms are most commonly seen in children between 6 and 24 months of age.

Atypical celiac disease is characterized by milder gastrointestinal symptoms. It is associated with extra-intestinal manifestations, such as iron deficiency anemia, osteoporosis, short stature, arthritis, infertility, peripheral neuropathy, hypertransaminasemia, and, in some cases, liver failure at the time of diagnosis.

Latent celiac disease occurs in patients who carry HLA-DQ2 and/or HLA-DQ8, with or without positive serology, and who have not yet developed villous atrophy but may have mild inflammation or immune activation. Patients in this subset may be asymptomatic or may have extra-intestinal manifestations.

Silent celiac disease is characterized by positive serology and villous atrophy in an otherwise asymptomatic patient. After undertaking a gluten-free diet some asymptomatic patients will notice improvement in different physical and psychological aspects of their life, such as improved appetite, reduced fatigue, or fewer behavioral abnormalities.

In some embodiments, a modified gluten polypeptide of the disclosure, such as a modified gluten polypeptide produced according to the methods provided herein, or a modified gluten polypeptide present in or obtained from a modified plant, plant tissue, plant organ, plant part, or plant cell of the disclosure, has a reduced ability to elicit one or more symptoms of Celiac disease (e.g., of any subtype), e.g., upon ingestion of the modified gluten polypeptide by an individual, e.g., as compared to an unmodified gluten polypeptide. In some embodiments, ingestion by an individual of the modified gluten polypeptide of the disclosure results in a reduction in the severity, frequency, and/or seriousness of one or more symptoms of Celiac disease by at least about 5%, at least about 10%, at least about 20%, at least about 30%, at least about 40%, at least about 50%, at least about 60%, at least about 70%, at least about 80%, at least about 90%, at least about 95%, at least about 99%, or 100%, e.g., as compared to the severity, frequency, and/or seriousness of the one or more symptoms upon ingestion of an unmodified gluten polypeptide.

### Dermatitis Herpetiformis

Dermatitis herpetiformis is a skin manifestation of celiac disease presenting with blistering rash and pathognomonic cutaneous IgA deposits. The predominant symptoms include, without limitation, rash, intense itching, and burning. The rash has a characteristic symmetrical distribution. The elbows and upper forearms are affected in more than 90% of patients. Other sites commonly involved are the buttocks, knees, shoulders, sacrum, face, scalp, neck and trunk. Celiac-type villous atrophy in the upper small intestinal mucosa is found in 65% to 75% of patients with dermatitis herpetiformis. Even in patients with apparently normal biopsies, subtle changes in the mucosa, such as an increased number of intraepithelial lymphocytes, indicate gluten sensitization. Dermatitis herpetiformis patients may show the same array of manifestations or symptoms as patients with celiac disease (autoimmune diseases, iron-deficient anemia, osteoporosis and malignancy). Dermatitis herpetiformis patients are generally put on a gluten-free diet because the rash of dermatitis herpetiformis is gluten sensitive.

In some embodiments, a modified gluten polypeptide of the disclosure, such as a modified gluten polypeptide produced according to the methods provided herein, or a modified gluten polypeptide present in or obtained from a modified plant, plant tissue, plant organ, plant part, or plant cell of the disclosure, has a reduced ability to elicit one or more symptoms of dermatitis herpetiformis, e.g., upon ingestion of the modified gluten polypeptide by an individual, e.g., as compared to an unmodified gluten polypeptide. In some embodiments, ingestion by an individual of the modified gluten polypeptide of the disclosure results in a reduction in the severity, frequency, and/or seriousness of one or more symptoms of dermatitis herpetiformis by at least about 5%, at least about 10%, at least about 20%, at least about 30%, at least about 40%, at least about 50%, at least about 60%, at least about 70%, at least about 80%, at least about 90%, at least about 95%, at least about 99%, or 100%, e.g., as compared to the severity, frequency, and/or seriousness of the one or more symptoms upon ingestion of an unmodified gluten polypeptide.

### Gluten Ataxia

Gluten ataxia is defined as otherwise idiopathic sporadic ataxia with positive serological markers for gluten sensitization. Like celiac disease, it is an autoimmune disease characterized by damage to the cerebellum resulting in ataxia. Gluten ataxia patients typically have high titer anti-gliadin antibodies. Widespread deposition of transglutaminase antibodies has been found around brain vessels in patients with gluten ataxia. Gluten ataxia usually presents with pure cerebellar ataxia or, rarely, ataxia in combination with myoclonus, palatal tremor or opsoclonus myoclonus. Gluten ataxia is usually of insidious onset with a mean age at onset of 53 years. Many patients will have evidence of enteropathy on intestinal biopsy. Patients positive for anti-gliadin antibodies or anti-tTG antibodies with no alternative cause for their ataxia are typically put on a strict gluten-free diet with regular follow-up.

In some embodiments, a modified gluten polypeptide of the disclosure, such as a modified gluten polypeptide produced according to the methods provided herein, or a modified gluten polypeptide present in or obtained from a modified plant, plant tissue, plant organ, plant part, or plant cell of the disclosure, has a reduced ability to elicit one or more symptoms of gluten ataxia, e.g., upon ingestion of the modified gluten polypeptide by an individual, e.g., as compared to an unmodified gluten polypeptide. In some embodiments, ingestion by an individual of the modified gluten polypeptide of the disclosure results in a reduction in the severity, frequency, and/or seriousness of one or more symptoms of gluten ataxia by at least about 5%, at least about 10%, at least about 20%, at least about 30%, at least about 40%, at least about 50%, at least about 60%, at least about 70%, at least about 80%, at least about 90%, at least about 95%, at least about 99%, or 100%, e.g., as compared to the severity, frequency, and/or seriousness of the one or more symptoms upon ingestion of an unmodified gluten polypeptide.

### Gluten Sensitivity

Gluten sensitivity, also referred to as non-celiac gluten sensitivity or gluten-intolerance, is generally characterized as a functional, morphological and immunological disorder that lacks all of the features of celiac disease, but nevertheless responds to gluten exclusion. Gluten sensitivity is distinct from celiac disease and is not accompanied by anti-tTG autoantibodies or other autoimmune comorbidities. The small intestine of gluten sensitivity patients is typically normal. The symptoms of gluten sensitivity may resemble those associated with celiac disease but with a prevalence of extra-intestinal symptoms, such as behavioral changes, bone or joint pain, muscle cramps, leg numbness, weight loss and chronic fatigue. There are no laboratory biomarkers specific for gluten sensitivity. Usually the diagnosis is based on exclusion criteria; an elimination diet of gluten-containing foods followed by an open challenge is most often used to evaluate whether health improves with the elimination or reduction of gluten from the patient's diet.

In some embodiments, a modified gluten polypeptide of the disclosure, such as a modified gluten polypeptide produced according to the methods provided herein, or a modified gluten polypeptide present in or obtained from a modified plant, plant tissue, plant organ, plant part, or plant cell of the disclosure, has a reduced ability to elicit one or more symptoms of gluten sensitivity, e.g., upon ingestion of the modified gluten polypeptide by an individual, e.g., as compared to an unmodified gluten polypeptide. In some embodiments, ingestion by an individual of the modified gluten polypeptide of the disclosure results in a reduction in the severity, frequency, and/or seriousness of one or more symptoms of gluten sensitivity by at least about 5%, at least about 10%, at least about 20%, at least about 30%, at least about 40%, at least about 50%, at least about 60%, at least about 70%, at least about 80%, at least about 90%, at least about 95%, at least about 99%, or 100%, e.g., as compared to the severity, frequency, and/or seriousness of the one or more symptoms upon ingestion of an unmodified gluten polypeptide.

### Gluten Toxicity

Toxicity upon ingestion of gluten polypeptides, e.g., gliadins and glutenins, can stem from cytotoxic or immunological mechanisms or a combination of cytotoxic and immunological mechanisms. Symptoms associated with gluten toxicity can include, without limitation, inflammation, autoimmune reactions, gastrointestinal symptoms such as diarrhea, steatorrhea, abdominal distension, weight loss, anemia, osteoporosis, arthritis, infertility, peripheral neuropathy, liver failure, and depression.

In some embodiments, a modified gluten polypeptide of the disclosure, such as a modified gluten polypeptide produced according to the methods provided herein, or a modified gluten polypeptide present in or obtained from a modified plant, plant tissue, plant organ, plant part, or plant cell of the disclosure, has a reduced ability to elicit one or more symptoms of gluten toxicity, e.g., upon ingestion of the modified gluten polypeptide by an individual, e.g., as compared to an unmodified gluten polypeptide. In some embodiments, ingestion by an individual of the modified gluten polypeptide of the disclosure results in a reduction in the severity, frequency, and/or seriousness of one or more symptoms of gluten toxicity by at least about 5%, at least about 10%, at least about 20%, at least about 30%, at least about 40%, at least about 50%, at least about 60%, at least about 70%, at least about 80%, at least about 90%, at least about 95%, at least about 99%, or 100%, e.g., as compared to the severity, frequency, and/or seriousness of the one or more symptoms upon ingestion of an unmodified gluten polypeptide.

### Gluten Properties

Gluten polypeptides confer water absorption capacity, cohesivity, viscosity, structure, appearance, mouth-feel, and elasticity on products, such as dough or baked goods (e.g., leavened bread, unleavened bread, bagels, crusts, pastries, cookies, crackers, and the like). In some embodiments, a modified gluten polypeptide of the disclosure, such as a modified gluten polypeptide produced according to the methods provided herein, or a modified gluten polypeptide present in or obtained from a modified plant, plant tissue, plant organ, plant part, or plant cell of the disclosure, has properties when used in products such as dough or baked goods (e.g., water absorption capacity, cohesivity, viscosity, structure, appearance, mouth-feel, and elasticity) that are substantially the same as the properties of an unmodified gluten polypeptide.

In some embodiments, a modified gluten polypeptide of the disclosure, such as a modified gluten polypeptide produced according to the methods provided herein, or a modified gluten polypeptide present in or obtained from a modified plant, plant tissue, plant organ, plant part, or plant cell of the disclosure, confers water absorption capacity on products (e.g., dough or baked goods, such as leavened bread, unleavened bread, bagels, crusts, pastries, cookies, crackers, and the like), that is substantially the same as the water absorption capacity conferred on products (e.g., dough or baked goods, such as leavened bread, unleavened bread, bagels, crusts, pastries, cookies, crackers, and the like) by an unmodified gluten polypeptide.

In some embodiments, a modified gluten polypeptide of the disclosure, such as a modified gluten polypeptide produced according to the methods provided herein, or a modified gluten polypeptide present in or obtained from a modified plant, plant tissue, plant organ, plant part, or plant cell of the disclosure, confers cohesivity on products (e.g., dough or baked goods, such as leavened bread, unleavened bread, bagels, crusts, pastries, cookies, crackers, and the like), that is substantially the same as the cohesivity conferred on products (e.g., dough or baked goods, such as leavened bread, unleavened bread, bagels, crusts, pastries, cookies, crackers, and the like) by an unmodified gluten polypeptide.

In some embodiments, a modified gluten polypeptide of the disclosure, such as a modified gluten polypeptide produced according to the methods provided herein, or a modified gluten polypeptide present in or obtained from a modified plant, plant tissue, plant organ, plant part, or plant cell of the disclosure, confers viscosity on products (e.g., dough or baked goods, such as leavened bread, unleavened bread, bagels, crusts, pastries, cookies, crackers, and the like), that is substantially the same as the viscosity conferred on products (e.g., dough or baked goods, such as leavened bread, unleavened bread, bagels, crusts, pastries, cookies, crackers, and the like) by an unmodified gluten polypeptide.

In some embodiments, a modified gluten polypeptide of the disclosure, such as a modified gluten polypeptide produced according to the methods provided herein, or a modified gluten polypeptide present in or obtained from a modified plant, plant tissue, plant organ, plant part, or plant cell of the disclosure, confers structure on products (e.g., dough or baked goods, such as leavened bread, unleavened bread, bagels, crusts, pastries, cookies, crackers, and the like), that is substantially the same as the structure conferred on products (e.g., dough or baked goods, such as leavened bread, unleavened bread, bagels, crusts, pastries, cookies, crackers, and the like) by an unmodified gluten polypeptide.

In some embodiments, a modified gluten polypeptide of the disclosure, such as a modified gluten polypeptide produced according to the methods provided herein, or a modified gluten polypeptide present in or obtained from a modified plant, plant tissue, plant organ, plant part, or plant cell of the disclosure, confers appearance on products (e.g., dough or baked goods, such as leavened bread, unleavened bread, bagels, crusts, pastries, cookies, crackers, and the like), that is substantially the same as the appearance conferred on products (e.g., dough or baked goods, such as leavened bread, unleavened bread, bagels, crusts, pastries, cookies, crackers, and the like) by an unmodified gluten polypeptide.

In some embodiments, a modified gluten polypeptide of the disclosure, such as a modified gluten polypeptide produced according to the methods provided herein, or a modified gluten polypeptide present in or obtained from a modified plant, plant tissue, plant organ, plant part, or plant cell of the disclosure, confers mouth-feel on products (e.g., dough or baked goods, such as leavened bread, unleavened bread, bagels, crusts, pastries, cookies, crackers, and the like), that is substantially the same as the mouth-feel conferred on products (e.g., dough or baked goods, such as leavened bread, unleavened bread, bagels, crusts, pastries, cookies, crackers, and the like) by an unmodified gluten polypeptide.

In some embodiments, a modified gluten polypeptide of the disclosure, such as a modified gluten polypeptide produced according to the methods provided herein, or a modified gluten polypeptide present in or obtained from a modified plant, plant tissue, plant organ, plant part, or plant cell of the disclosure, confers elasticity on products (e.g., dough or baked goods, such as leavened bread, unleavened bread, bagels, crusts, pastries, cookies, crackers, and the like), that is substantially the same as the elasticity conferred on products (e.g., dough or baked goods, such as leavened bread, unleavened bread, bagels, crusts, pastries, cookies, crackers, and the like) by an unmodified gluten polypeptide.

### Compositions of the Disclosure

### Orally Consumable Products

Modified gluten polypeptides of the disclosure, e.g., a modified gluten polypeptide produced according to the methods provided herein, or a modified gluten polypeptide present in or obtained from a modified plant, plant tissue, plant organ, plant part, or plant cell of the disclosure, may be formulated into an orally consumable product. As used herein, the term “orally consumable product(s)” refers to edible substances which are contacted with the mouth of man or animal, including substances that are taken into and subsequently ejected from the mouth, and substances which are drunk, eaten, swallowed, or otherwise ingested; and that are safe for human or animal consumption when used in a generally acceptable range of concentrations.

Orally consumable products of the present disclosure may take various forms. An orally consumable product of the present disclosure may include, for example, a foodstuff composition, a beverage product, a dietary supplement, a nutraceutical, an edible gel mix, an edible gel composition, and a pharmaceutical composition.

Orally consumable products may also be formulated into a granulated powder, a soft gel composition, and a flash dissolve composition.

Orally consumable products may contain one or more sweeteners. The one or more sweeteners may already be present in the orally consumable product or may be added to the orally consumable product, or one or more compounds or ingredients used to make the orally consumable product. In certain embodiments, the one or more sweeteners may include, for example, natural sweeteners, and artificial or synthetic sweeteners. Suitable sweeteners and combinations of sweeteners may be selected for the desired nutritional characteristics, taste profile, mouthfeel, and other organoleptic factors. In some embodiments, the one or more sweeteners include high intensity sweeteners and/or natural high intensity sweeteners, including, for example, stevia extracts, steviol glycosides, steviosides, rebaudioside A, rebaudioside B, rebaudioside C, rebaudioside D, rebaudioside F, dulcoside A, rubusosides, steviolbiosides, sucrose, high fructose corn syrup, fructose, fructooligosaccharides, glucose, xylose, arabinose, rhamnose, erythritol, xylitol, mannitol, sorbitol, inositol, AceK, aspartame, neotame, oligofructose, sucralose, saccharine, naringin dihydrochalcone (NarDHC), neohesperidin dihydrochalcone (NDHC), rubusoside, mogroside IV, siamenoside I, mogroside V, monatin, thaumatin, monellin, brazzein, L-alanine, glycine, Lo Han Guo, hernandulcin, phyllodulcin, trilobtain, and combinations thereof.

Orally consumable products of the present disclosure may contain one or more additives. The one or more additives may be present to add or enhance one or more characteristics of the orally consumable product, such as flavor, texture, aroma, color, shelf-life, etc. The one or more additives may already be present in the orally consumable product or may be added to the orally consumable product, or one or more compounds or ingredients used to make the orally consumable product. The orally consumable product may contain any suitable additive known in the art. Examples of suitable additives include, for example, carbohydrates, polyols, amino acids or salts thereof, poly-amino acids or salt thereof, sugar acids or salts thereof, nucleotides, organic acids, inorganic acids, organic salts, organic acid salts, organic base salts, inorganic salts, bitter compounds, flavorants, flavoring ingredients, astringent compounds, proteins, protein hydrolysates, surfactants, emulsifiers, flavonoids, alcohols, polymers, preservatives, thickening agents, food colorings, and combinations thereof.

### Foodstuff Compositions

Modified gluten polypeptides of the disclosure, e.g., a modified gluten polypeptide produced according to the methods provided herein, or a modified gluten polypeptide present in or obtained from a modified plant, plant tissue, plant organ, plant part, or plant cell of the disclosure, may be formulated into foodstuff compositions. As used herein, “foodstuff composition(s)” refers to any solid or liquid ingestible material that may, but need not, have a nutritional value and is intended for consumption by man or animal.

Examples of suitable foodstuff compositions may include, for example, beverages (both carbonated and non-carbonated), such as coffee, teas, herbal teas, fruit drinks, soft drinks (e.g., colas), and the like; confectionary compositions, such as candies, mints, fruit flavored drops, cocoa products, chocolates, and the like; condiments, such as ketchup, mustard, mayonnaise, and the like; chewing gums; cereal compositions; baked goods, such as breads, cakes, pies, cookies, and the like; dairy products, such as milk, cheese, cream, ice cream, sour cream, yoghurt, sherbet, and the like; tabletop sweetener compositions; soups; stews; convenience foods; meats, such as ham, bacon, sausages, jerky, and the like; gelatins and gelatin-like products such as jams, jellies, preserves, and the like; fruits; vegetables; egg products; icings; syrups including molasses; snacks; nut meats and nut products; and animal feed.

Foodstuff compositions may also include herbs, spices and seasonings, natural and synthetic flavors, and flavor enhancers. In some embodiments, foodstuff compositions include, for example, prepared packaged products, such as dietetic sweeteners, liquid sweeteners, granulated flavor mixes which upon reconstitution with water provide non-carbonated drinks, instant pudding mixes, instant coffee and tea, coffee whiteners, malted milk mixes, pet foods, livestock feed, and materials for baking applications, such as powdered baking mixes for the preparation of breads, cookies, cakes, pancakes, donuts and the like.

### Beverage Products

Modified gluten polypeptides of the disclosure, e.g., a modified gluten polypeptide produced according to the methods provided herein, or a modified gluten polypeptide present in or obtained from a modified plant, plant tissue, plant organ, plant part, or plant cell of the disclosure, may be formulated into beverage products. Beverage products of the present disclosure include both carbonated and non-carbonated beverage products. Examples of suitable beverage products include, for example, soft drinks, fountain beverages, frozen beverages, ready-to-drink beverages, coffee, teas, dairy beverages, powdered soft drinks, liquid concentrates, flavored water, enhanced water, fruit juices, fruit juice flavored drinks, sport drinks, energy drinks, and alcoholic beverages, such as beers, wines, and liquors.

In some embodiments, a beverage product of the present disclosure includes one or more beverage ingredients including, for example, acidulants, fruit juices and/or vegetable juices, pulp, etc., flavorings, coloring, preservatives, vitamins, minerals, electrolytes, erythritol, tagatose, glycerine, and carbon dioxide. Such beverage products may be provided in any suitable form, such as a beverage concentrate or a carbonated, ready-to-drink beverage.

In certain embodiments, beverage products of the present disclosure may have any of numerous different specific formulations or constitutions. The formulation of a beverage product of the present disclosure may vary to a certain extent, depending upon such factors as the product's intended market segment, its desired nutritional characteristics, flavor profile, and the like. For example, in certain embodiments, it will generally be an option to add further ingredients to the formulation of a particular beverage product. For example, sweeteners, flavorings, electrolytes, vitamins, fruit juices or other fruit products, tastents, masking agents and the like, flavor enhancers, and/or carbonation typically may be added to any such formulations to vary the taste, mouthfeel, nutritional characteristics, etc. In some embodiments, orally consumable products are formulated to exhibit a particular flavor(s). Flavors that may be used may include, for example, vanilla flavor, chocolate flavor, banana flavor, strawberry flavor, and various others that will be readily apparent to one of skill in the art. Exemplary additional flavorings include, for example, cola flavoring, citrus flavoring, and spice flavorings. In some embodiments, carbonation in the form of carbon dioxide may be added for effervescence. In other embodiments, preservatives may be added, depending upon the other ingredients, production technique, desired shelf life, etc. In certain embodiments, caffeine may be added. In some embodiments, the beverage product is a cola-flavored carbonated beverage, characteristically containing carbonated water, sweetener, kola nut extract and/or other flavoring, caramel coloring, one or more acids, and optionally other ingredients.

### Dietary Supplements and Nutraceuticals

Modified gluten polypeptides of the disclosure, e.g., a modified gluten polypeptide produced according to the methods provided herein, or a modified gluten polypeptide present in or obtained from a modified plant, plant tissue, plant organ, plant part, or plant cell of the disclosure, may be formulated into dietary supplements. As used herein, “dietary supplement(s)” refers to compounds intended to supplement the diet and provide nutrients, such as vitamins, minerals, fiber, fatty acids, amino acids, etc. that may be missing or may not be consumed in sufficient quantities in a diet. Any suitable dietary supplement known in the art may be used. Examples of suitable dietary supplements include, for example, nutrients, vitamins, minerals, fiber, fatty acids, herbs, botanicals, amino acids, and metabolites.

Modified gluten polypeptides of the disclosure, e.g., a modified gluten polypeptide produced according to the methods provided herein, or a modified gluten polypeptide present in or obtained from a modified plant, plant tissue, plant organ, plant part, or plant cell of the disclosure, may also be formulated into nutraceuticals. As used herein, “nutraceutical(s)” refers to compounds, which includes any food or part of a food that may provide medicinal or health benefits, including the prevention and/or treatment of disease or disorder (e.g., fatigue, insomnia, effects of aging, memory loss, mood disorders, cardiovascular disease and high levels of cholesterol in the blood, diabetes, osteoporosis, inflammation, autoimmune disorders, etc.). Any suitable nutraceutical known in the art may be used. In some embodiments, nutraceuticals can be used as supplements to food and beverages and as pharmaceutical formulations for enteral or parenteral applications which may be solid formulations, such as capsules or tablets, or liquid formulations, such as solutions or suspensions.

In some embodiments, dietary supplements and nutraceuticals may further contain protective hydrocolloids (such as gums, proteins, modified starches), binders, film-forming agents, encapsulating agents/materials, wall/shell materials, matrix compounds, coatings, emulsifiers, surface active agents, solubilizing agents (oils, fats, waxes, lecithins, etc.), adsorbents, carriers, fillers, co-compounds, dispersing agents, wetting agents, processing aids (solvents), flowing agents, taste-masking agents, weighting agents, jellifying agents, gel-forming agents, antioxidants and antimicrobials.

### Edible Gel Mixes and Gel Compositions

Modified gluten polypeptides of the disclosure, e.g., a modified gluten polypeptide produced according to the methods provided herein, or a modified gluten polypeptide present in or obtained from a modified plant, plant tissue, plant organ, plant part, or plant cell of the disclosure, may be formulated into gel mixes and gel compositions. As used herein, a “gel” refers to a colloidal system in which a network of particles spans the volume of a liquid medium. Although gels mainly are composed of liquids, and thus exhibit densities similar to liquids, gels have the structural coherence of solids due to the network of particles that spans the liquid medium. For this reason, gels generally appear to be solid, jelly-like materials. Gels that can be eaten are referred to as “edible gel compositions.” Edible gel compositions typically are eaten as snacks, as desserts, as a part of staple foods, or along with staple foods. Examples of suitable edible gel compositions include, for example, gel desserts, puddings, jams, jellies, pastes, trifles, aspics, marshmallows, gummy candies, and the like. In some embodiments, edible gel mixes generally are powdered or granular solids to which a fluid may be added to form an edible gel composition. Examples of suitable fluids include, for example, water, dairy fluids, dairy analogue fluids, juices, alcohol, alcoholic beverages, and combinations thereof. Examples of suitable dairy fluids include, for example, milk, cultured milk, cream, fluid whey, and mixtures thereof. Examples of suitable dairy analogue fluids include, for example, soy milk and non-dairy coffee whitener.

As used herein, the term “gelling ingredient” refers to any material that can form a colloidal system within a liquid medium. Examples of suitable gelling ingredients include, for example, gelatin, alginate, carageenan, gum, pectin, konjac, agar, food acid, rennet, starch, starch derivatives, and combinations thereof. It is well known to those having ordinary skill in the art that the amount of gelling ingredient used in an edible gel mix or an edible gel composition varies considerably depending on a number of factors including, for example, the particular gelling ingredient used, the particular fluid base used, and the desired properties of the gel.

Gel mixes and gel compositions of the present disclosure may be prepared by any suitable method known in the art. In some embodiments, edible gel mixes and edible gel compositions of the present disclosure may be prepared using other ingredients in addition to a modified gluten polypeptide of the present disclosure and the gelling agent. Examples of other suitable ingredients include, for example, a food acid, a salt of a food acid, a buffering system, a bulking agent, a sequestrant, a cross-linking agent, one or more flavors, one or more colors, and combinations thereof.

### Pharmaceutical Compositions

Modified gluten polypeptides of the disclosure, e.g., a modified gluten polypeptide produced according to the methods provided herein, or a modified gluten polypeptide present in or obtained from a modified plant, plant tissue, plant organ, plant part, or plant cell of the disclosure, may be formulated into pharmaceutical compositions. Any suitable pharmaceutical composition known in the art may be used. In certain embodiments, a pharmaceutical composition of the present disclosure contains a modified gluten polypeptide of the present disclosure and one or more pharmaceutically acceptable excipients. In some embodiments, pharmaceutical compositions of the present disclosure may be used to formulate pharmaceutical drugs containing one or more active agents that exert a biological effect. Accordingly, in some embodiments, pharmaceutical compositions of the present disclosure may contain one or more active agents that exert a biological effect. Suitable active agents are well known in the art (e.g., The Physician's Desk Reference). Such compositions can be prepared according to procedures well known in the art, for example, as described in Remington's Pharmaceutical Sciences, Mack Publishing Co., Easton, PA., USA.

Examples of suitable active agents include, for example, bronchodilators, anorexiants, antihistamines, nutritional supplements, laxatives, analgesics, anesthetics, antacids, H.sub.2-receptor antagonists, anticholinergics, antidiarrheals, demulcents, antitussives, antinauseants, antimicrobials, antibacterials, antifungals, antivirals, expectorants, anti-inflammatory agents, antipyretics, and mixtures thereof. In one embodiment, the active agent is selected from the group consisting of antipyretics and analgesics, e.g., ibuprofen, acetaminophen, or aspirin; laxatives, e.g., phenolphthalein dioctyl sodium sulfosuccinate; appetite depressants, e.g., amphetamines, phenylpropanolamine, phenylpropanolamine hydrochloride, or caffeine; antacidics, e.g., calcium carbonate; antiasthmatics, e.g., theophylline; antidiuretics, e.g., diphenoxylate hydrochloride; agents active against flatulence, e.g., simethecon; migraine agents, e.g., ergotaminetartrate; psychopharmacological agents, e.g., haloperidol; spasmolytics or sedatives, e.g., phenobarbitol; antihyperkinetics, e.g., methyldopa or methylphenidate; tranquilizers, e.g., benzodiazepines, hydroxinmeprobramates or phenothiazines; antihistaminics, e.g., astemizol, chloropheniramine maleate, pyridamine maleate, doxlamine succinate, bromopheniramine maleate, phenyltoloxamine citrate, chlorocyclizine hydrochloride, pheniramine maleate, and phenindamine tartrate; decongestants, e.g., phenylpropanolamine hydrochloride, phenylephrine hydrochloride, pseudoephedrine hydrochloride, pseudoephedrine sulfate, phenylpropanolamine bitartrate, and ephedrine; beta-receptor blockers, e.g., propanolol; agents for alcohol withdrawal, e.g., disulfiram; antitussives, e.g., benzocaine, dextromethorphan, dextromethorphan hydrobromide, noscapine, carbetapentane citrate, and chlophedianol hydrochloride; fluorine supplements, e.g., sodium fluoride; local antibiotics, e.g., tetracycline or cleocine; corticosteroid supplements, e.g., prednisone or prednisolone; agents against goiter formation, e.g., colchicine or allopurinol; antiepileptics, e.g., phenytoine sodium; agents against dehydration, e.g., electrolyte supplements; antiseptics, e.g., cetylpyridinium chloride; NSAIDs, e.g., acetaminophen, ibuprofen, naproxen, or salts thereof; gastrointestinal active agents, e.g., loperamide and famotidine; various alkaloids, e.g., codeine phosphate, codeine sulfate, or morphine; supplements for trace elements, e.g., sodium chloride, zinc chloride, calcium carbonate, magnesium oxide, and other alkali metal salts and alkali earth metal salts; vitamins; ion-exchange resins, e.g., cholestyramine; cholesterol-depressant and lipid-lowering substances; antiarrhythmics, e.g., N-acetylprocainamide; and expectorants, antibacterial agents such as ciprofloxacin, ofloxacin, and pefloxacin; antiepileptics such as zonisamide; macrolide antibiotics such as erythromycin; beta-lactam antibiotics such as penicillins and cephalosporins; psychotropic active substances such as chlorpromazine; active substances such as sulpyrine; and agents active against ulcers, such as cimetidine. In some embodiments, the pharmaceutical composition of the present invention contains at least one amino acid selected from the group consisting of glycine, L-alanine, L-arginine, L-aspartic acid, L-cystine, L-glutamic acid, L-glutamine, L-histidine, L-isoleucine, L-leucine, L-lysine, L-methionine, L-ornithine, L-phenylalanine, L-proline, L-serine, L-threonine, L-tryptophan, L-tyrosine, L-valine, creatine, and mixtures thereof.

In some embodiments, a pharmaceutical composition of the present disclosure is a liquid dosage form for oral administration including, for example, pharmaceutically acceptable emulsions, solutions, suspensions, syrups, and elixirs. In addition to the active compounds, the liquid dosage forms may contain inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethyl formamide, oils (such as cottonseed, groundnut, corn, germ, olive, castor, and sesame oils), glycerol, tetrahydrofurfuryl alcohol, polyethylene glycols and fatty acid esters of sorbitan, and mixtures thereof. Suspensions, in addition to the active compounds, may contain suspending agents as, for example, ethoxylated isostearyl alcohols, polyoxyethylene sorbitol and sorbitan esters, microcrystalline cellulose, aluminum metahydroxide, bentonite, agar-agar, and tragacanth, and mixtures thereof.

In other embodiments, pharmaceutical compositions of the present disclosure can be in the form of chewable tablets; orally disintegrating compositions; solid dosage forms, such as water and/or saliva activated effervescent granule; and film-shaped or wafer-shaped pharmaceutical compositions; gum base formulations having a medicament or agent and a modified gluten polypeptide of the present disclosure contained in a coating that surrounds the gum base formulation.

### Cosmetic or Skin Compositions

Modified gluten polypeptides of the disclosure, e.g., a modified gluten polypeptide produced according to the methods provided herein, or a modified gluten polypeptide present in or obtained from a modified plant, plant tissue, plant organ, plant part, or plant cell of the disclosure, may be formulated into cosmetic compositions or skin compositions. Examples of suitable cosmetic or skin compositions include, without limitation, shampoo, body wash, facial cleanser, face mask, bubble bath, intimate wash, bath oil, cleansing milk, micellar water, make-up remover, cleansing wipes, hair mask, perfume, soaps, liquid soap, shaving soap, shaving foam, cleansing foam, day cream, anti-ageing cream, body milk, body lotion, body mousse, face serum, eye cream, sunscreen lotion, sun cream, face cream, after-shave lotion, pre-shaving cream, depilatory cream, skin-whitening gel, self-tanning cream, anti-acne gel or cream, mascara, foundation, primer, concealer, blush, bronzer, blemish balm cream, eyeliner, night cream, eye brow gel, highlighter, lip stain, hand sanitizer, hair oil, nail varnish remover, conditioner, hair styling gel, hair styling cream, mouth wash, anti-frizz serum, scalp treatment, hair colorant, split end fluid, deodorant, antiperspirant, baby cream, insect repellent, hand cream, sunscreen gel, foot cream, exfoliator, body scrub, cellulite treatment, bar soap, cuticle cream, lip balm, hair treatment, eye shadow, hair peptide serum, bath additive, body mist, eau de toilette, mouthwash, toothpaste, lubricating gel, moisturizer, serum, toner, aqua sorbet, cream gel, styling mousse, dry shampoo, lip stick, lip gloss, hydro-alcoholic gel, body oil, shower milk, illuminator, lip crayon, hair spray, combing cream, and sunblock. In some embodiments, the composition is a hair shampoo, hair conditioner, or a hair peptide serum composition. In some embodiments, the composition is a soap composition, such as a liquid soap, bar soap, foaming soap, powdered soap, or shaving soap composition. In some embodiments, the composition is a lotion composition, such as skin lotion, body lotion, face lotion, sunscreen lotion, hand lotion, or shave lotion. In some embodiments, the composition is a hair peptide serum composition.

The cosmetic or skin compositions of the disclosure may further include one or more additional components, including, without limitation, waxes, emulsifiers, co-emulsifiers, solubilizers, cationic polymers, film formers, superfatting agents, refatting agents, foam stabilizers, stabilizers, active biogenic substances, preservatives, preservation boosting ingredients, anti-fungal substances, anti-dandruff agents, dyes or pigments, particulate substances, opacifiers, abrasives, absorbents, anticaking agents, bulking agents, pearlizing agents, oily substances, direct dyes, perfumes or fragrances, carriers, solvents or diluents, propellants, functional acids, active ingredients, skin-brightening agents, self-tanning agents, exfoliants, enzymes, anti-acne agents, deodorants and anti-perspirants, viscosity modifiers, thickening and gelling agents, pH adjusting agents, buffering agents, anti-oxidants, chelants, astringents, sunscreens, sun protection agents, UV filters, skin conditioning agents, emollients, humectants, occlusive agents, pediculocides, anti-foaming agents, flavouring agents, electrolytes, oxidizing agents and reducing agents.

### EXAMPLES

The invention will be more fully understood by reference to the following examples. They should not, however, be construed as limiting the scope of the invention. It is understood that the examples and embodiments described herein are for illustrative purposes only and that various modifications or changes in light thereof will be suggested to persons skilled in the art and are to be included within the spirit and purview of this application and scope of the appended claims.

Gluten is a complex mixture of related storage proteins of wheat grains, mainly gliadin and glutenin. Together, these families of proteins make up 85% of protein content in the wheat kernel and are referred as prolamins. These are characterized by their high glutamine (38%) and proline (20%) content. Some of the key differences between gluten proteins is derived from their structure and interaction, including their cysteine content, which gives them the ability to form strong covalent networks, and their molecular weight. See Biesiekierski, J. R. What is gluten?Journal of Gastroenterology and Hepatology 32, 78-81 (2017). Gliadins are further classified into subgroups by their primary structures into α, β, γ, and ω gliadins (for example, see Shewry, P. R. & Lookhart, G. L. Wheat gluten protein analysis. American Association of Cereal Chemists (2003) and Altenbach, S. B. et al. Reducing the Immunogenic Potential of Wheat Flour: Silencing of Alpha Gliadin Genes in a U.S. Wheat Cultivar. Frontiers in Plant Science 11 (2020)). The composition and distribution of gluten proteins vary among different wheat strains, which determine the unique properties of the gluten protein networks for food production.

Other cereals contain similar storage proteins such as the hordein in barley, secalin in rye and avenins in oats. The “gluten” protein content in these grains varies between cereals. For instance, while hordeins constitute 30% of the proteins in the grain (for example, see Tanner, G. J. et al. Hordein Accumulation in Developing Barley Grains. Frontiers in Plant Science 10 (2020)), avenins only make up 10% of the oat grain protein (for example, see Howdle, P. D. in Encyclopedia of Food Sciences and Nutrition (Second Edition) (ed Benjamin Caballero), 987-994 (Academic Press, 2003)). These are considerably smaller compared to the high proportion of gluten proteins in wheat.

Gluten-related disorders (GRD) have become increasingly common in the western world. Celiac disease (CeD) is the most well studied affliction caused by gluten consumption, where fragments of the gluten proteins trigger a T-cell mediated autoimmune response causing damage to the intestine's lining (for example, see Elli, L. et al. Diagnosis of gluten related disorders: Celiac disease, wheat allergy and non-celiac gluten sensitivity. World J Gastroenterol 21, 7110-7119 (2015)). Unlike CeD, which requires human leukocyte antigen variants (DQ2 or DQ8 alleles) (for example, see Sollid, L. M. et al. Evidence for a primary association of celiac disease to a particular HLA-DQ alpha/beta heterodimer. J Exp Med 169, 345-350 (1989); Megiorni, F. et al. HLA-DQ and susceptibility to celiac disease: evidence for gender differences and parent-of-origin effects. Am J Gastroenterol 103, 997-1003 (2008); and Kaukinen, K. et al. HLA-DQ typing in the diagnosis of celiac disease. Am J Gastroenterol 97, 695-699 (2002)), an understanding of the underlying causes of other GRDs such as gluten allergy (GA) and non-celiac gluten sensitivity (NCGS) remains elusive in the field (for example, see Barbaro, M. R. et al. Recent advances in understanding non-celiac gluten sensitivity. F1000Res 7, F1000 Faculty Rev-1631 (2018); and Elli, L. et al. (2015)). Given the rise of GRDs, which affect about 6 times more of the population than CeD, and given that the average Western diet is thought to consist of 5-20 grams of gluten per day (for example, see Hoppe, C. et al. Intake and sources of gluten in 20- to 75-year-old Danish adults: a national dietary survey. European Journal of Nutrition 56, 107-117 (2017); and Biesiekierski, J. R. (2017)), it is important to identify how gluten consumption triggers these gastrointestinal afflictions and to find a preventative or curative solution for these afflictions. However, a clear mechanistic understanding of what precisely drives the inflammatory processes behind GA and NCGS afflictions is lacking.

Both gluten allergy (GA) and non-celiac gluten sensitivity (NCGS) present symptoms associated with inflammation or immune activation upon ingestion of gluten. As a food allergen, in GA, gluten can affect the skin, the gastrointestinal tract, or the respiratory tract, with symptoms including urticaria, angioedema, bronchial obstruction, nausea, diarrhea, abdominal pain, and even systematic anaphylaxis in severe cases. While diagnosis of NCGS is not well defined yet given a lack of markers and understanding of its pathogenetic mechanisms, the most common symptoms for this affliction include abdominal bloating and pain, diarrhea, nausea and constipation. See, Elli, L. et al. Diagnosis of gluten related disorders: Celiac disease, wheat allergy and non-celiac gluten sensitivity. World J Gastroenterol 21, 7110-7119 (2015). However, in addition to gastrointestinal (GI) symptoms, patients often report a “foggy mind”, reduction of mnemonic capabilities, and lack of well-being, as well as tiredness, headache, anxiety, numbness, joint/muscle pain, and skin rash/dermatitis (Barbaro et al., Recent advances in understanding non-celiac gluten sensitivity. F1000Res 7, F1000 Faculty Rev-1631 (2018)). Such a collection of symptoms is cognate to other autoimmune diseases such as lupus and rheumatoid arthritis. Clearly, gluten, or fragments thereof, can function either directly or indirectly as immunomodulatory agents. Moreover, that the affected tissues are diverse suggests that immune pathways common to multiple systems of the body are involved.

Previously, a 33-mer peptide from α-gliadin was identified as a potent inducer of T-cells from patients with celiac disease (CeD) (Shan, L. et al. Structural basis for gluten intolerance in celiac sprue. Science 297, 2275-2279 (2002)). However, this immunomodulatory activity seems to be specific to individuals with human leukocyte antigen DQ2 or DQ8 alleles. In contrast, GA and NCGS afflict a much broader group of people (with a patient population that is ˜5× larger), indicating the presence of other immunomodulatory components in gluten that play a role in triggering a more general pathogenic mechanism.

In the arena of autoimmune diseases such as psoriasis, lupus, and rheumatoid arthritis, which are distinct from GA and NCGS, a drastic amplification of immune activation can be achieved when host antimicrobial peptides (AMPs) form structured complexes with immune ligands such as dsDNA and dsRNA, thereby enabling multivalent binding to arrays of Toll-Like Receptors (TLRs) and resultant amplified innate immune responses (Lee, E. Y. et al. Functional Reciprocity of Amyloids and Antimicrobial Peptides: Rethinking the Role of Supramolecular Assembly in Host Defense, Immune Activation, and Inflammation. Frontiers in Immunology 11, (2020); Lee, E. Y. et al. Helical antimicrobial peptides assemble into protofibril scaffolds that present ordered dsDNA to TLR9. Nature Communications 10, 1012, (2019); and Schmidt, N. W. et al. Liquid-crystalline ordering of antimicrobial peptide-DNA complexes controls TLR9 activation. Nature Materials 14, 696-700 (2015)). However, whether exogenous AMP mimics could be present in gluten or gluten-related proteins, bind and organize nucleic acids into ordered complexes like host AMPs, and amplify immune responses in a similar fashion, was unknown. Indeed, given the protease rich environment of the GI tract and the intrinsic presence of its microbiome, gluten fragments are found in the GI, as are pathogenic ligands that are connected to the innate immune system, such as dsDNA and dsRNA.

The Examples below describe the characterization of a potent mechanism of gluten-associated inflammation, its protein sequence dependence, and modifications of gluten that can suppress this mechanism of inflammation.

### Example 1: Discovery and Characterization of Immunoactive Peptide Fragments from Gluten

This Example describes the identification and characterization of immunoactive peptide fragments derived from gluten proteins.

### Materials and Methods

To discover candidate gluten fragments with the potential to associate with pathogen ligands, machine learning routines were trained with a curated library of antimicrobial peptides (AMPs) and subsequently experimentally validated. Nine different wheat α-gliadin isoforms from UniProtKB/Swiss-Prot (Primary accession: P18573, P04721, P04722, P04723, P04724, P04725, P04726, P04727, P04728) were scanned, and probability scores were generated to assess the likelihood of these isoforms forming organized complexes with pathogen associated molecular patterns (PAMPs). The average probability at each amino acid position (calculated from a window-scan scoring over the full sequence-window size of 19 amino acids) was then analyzed.

### Results

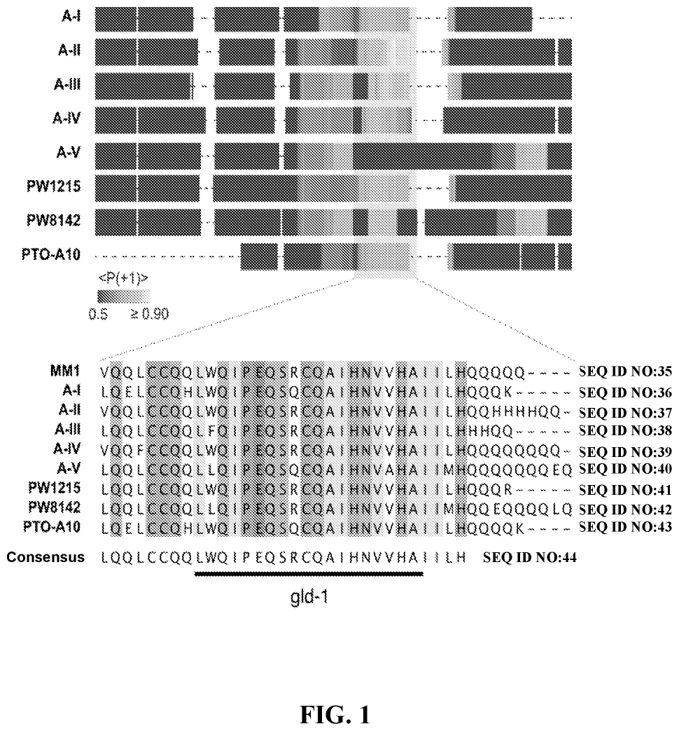

A subset of the analyzed AMPs had physical characteristics that led to the capacity to form organized complexes with PAMPs to activate inflammation driven by the innate immune system. Gliadin and glutenin from wheat were scanned, and candidate fragments were found in both prolamins. However, a fragment in α-gliadin (distal from the 33-mer identified by Shan, L. et. al. Structural basis for gluten intolerance in celiac sprue. Science 297, 2275-2279 (2002)) was identified by its high probability score as the most prominent candidate. This peptide spanned the non-repetitive region 1 (NRR1) of α-gliadin and was highly conserved across its isoforms (Clustal Omega alignment), as shown in FIG. 1.

A consensus sequence of these isoforms was identified using EMBOSS Cons (FIG. 1); this sequence is listed as “cons_agld_nrr1” in Table 2 below. However, an optimized 19-mer peptide was curated based on its physicochemical properties from cons_agld_nrr1 and is listed as “gld-1” in Table 2 (FIG. 1). This finding is particularly relevant because an 8 amino acid sub-fragment of gld-1 previously was shown to survive gastric plus pancreatic protease mediated digestion under physiological conditions in vitro (Shan, L. et. al. Structural basis for gluten intolerance in celiac sprue. Science 297, 2275-2279 (2002)), which means that this region may survive protease degradation as it travels through the GI tract and encounters the microbiome. Furthermore, this inflammatory peptide sequence exhibited scores similar to the inflammatory fragment previously identified as the causative sequence for C. difficile-driven colitis (Chen, X. et al. Clostridioides difficile Toxin A Remodels Membranes and Mediates DNA Entry Into Cells to Activate Toll-Like Receptor 9 Signaling. Gastroenterology 159, 2181-2192.e2181 (2020)).

TABLE 2
List of wild type, consensus, and mutant gluten peptide sequences.

| Name | Amino Acid Sequence | SEQ ID NO |
|---|---|---|
| cons- | ILQQILQQQLIPCRDVVLQQHNIAHGSSQVLQQSTYQLLQQ | 28 |
| agld- | LCCQQLWQIPEQSRCQAIHNVVHAIILHQQQQQ | |
| nrr1 | | |
| gld-1 | LWQIPEQSRCQAIHNVVHA | 29 |
| gld-1A | LAAQLWQIPEQSRAQAIHNVVH | 30 |
| gld-2 | LWQIPEQSQCQAIHNVVHA | 31 |
| gld-3 | LWQIPEQSQCQAIHNVVQA | 32 |
| gld-4 | RCCQQLRDVSAKCRSVAVSQVAR | 33 |
| gld-4A | RAAQQLRDVSAKARSVAVSQVAR | 34 |

Other gliadins, such as γ-gliadin, also contained a parallel NRR1 sequence similar to α-gliadin, with candidate sequences with immunomodulatory activity. However, α-gliadin demonstrated the highest scores for potential immunomodulation activity. Another cognate region was found in high molecular weight glutenin, although with lower propensity for immunomodulation compared to gld-1; this peptide is identified as “gld-4” in Table 2.

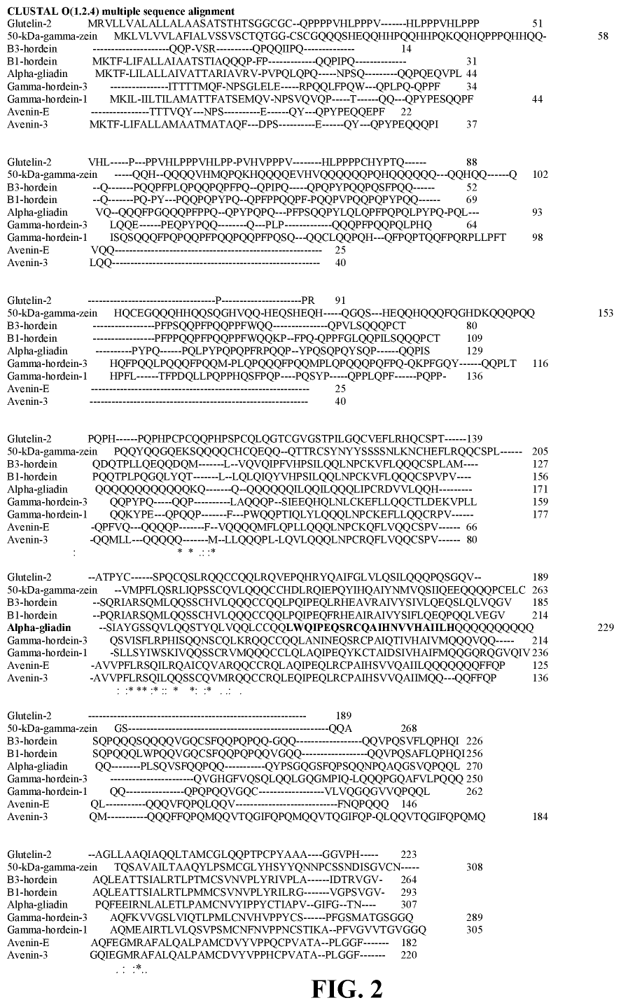

Other gluten related cereals were then scanned for candidate immunomodulatory peptides, including hordeins from barley, avenins from oats, and gluten-related proteins in corn (glutelin-2 and γ-zein 50 k). Sequence alignments of hordeins from barley (γ-hordein 1 and 3, B1- and B3-hordein), avenins from oats (avenin-3 and -E), and gluten-related proteins from corn (glutelin-2 and γ-zein 50 k) were performed. Similar candidate sequences were identified, predominantly in hordeins and avenins, with sequence alignments coinciding with the identified fragment in the NRR1 of α-gliadin (FIG. 2). In contrast, although the gluten-related proteins from corn aligned with the NRR1 of α-gliadin, the region lacked prominent candidates for immunomodulation.

### Conclusions

The gluten peptide fragments identified above are candidates for immune activation. Example 2, below, describes results of experiments showing that such peptide fragments can result in strong innate immune activation.

### Example 2: Characterization of Immunomodulatory Properties of Gluten Fragments

This Example describes the results of experiments that characterized the immunomodulatory properties of gluten peptide fragments identified in Example 1, above, as well as engineered mutants of such fragments with strongly attenuated immune activity.

Gluten Peptide Fragments Complex with Double Stranded DNA

Double stranded DNA (dsDNA) is an example of a PAMP that is readily available in the gastrointestinal tract (e.g., from food or the gut microbiome). To assess the ability of gluten derived peptide gld-1 to bind dsDNA, Escherichia coli dsDNA was incubated with gld-1, and the formation of an ordered dsDNA-gld-1 complex was assessed.

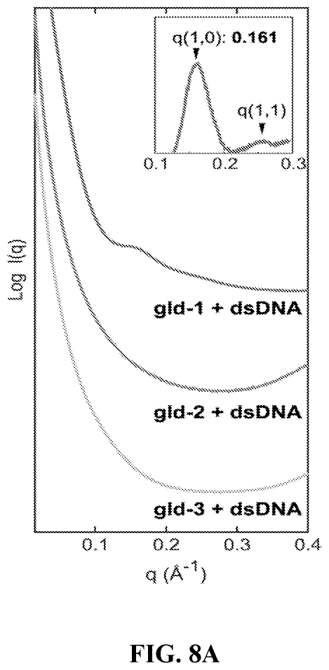

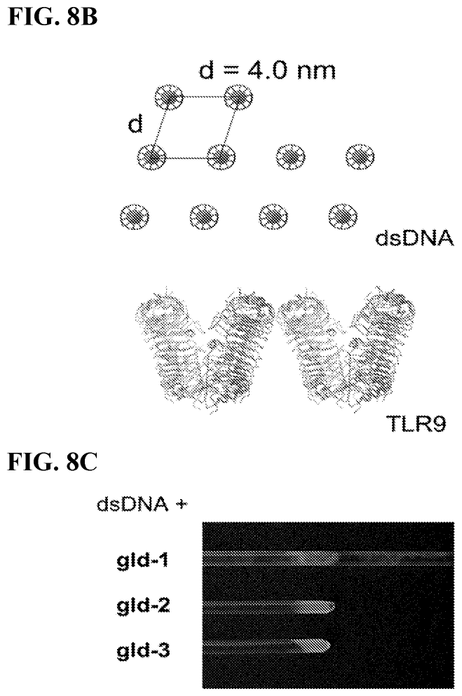

Small Angle X-ray Scattering (SAXS) measurements of the complex produced Bragg reflections at q₁₀=1.61 and q₁₁=2.55 nm⁻¹. The peaks corresponded to a rhombic columnar lattice with inter dsDNA spacing, d=4.0 nm, and tilt angle, γ=104.6° (FIGS. 8A-8B), which was in the range of optimal values that has been shown to amplify TLR9 activation in a deterministic manner (Lee, E. Y. et al. Helical antimicrobial peptides assemble into protofibril scaffolds that present ordered dsDNA to TLR9. Nature Communications 10, 1012, (2019); Schmidt, N. W. et al. Liquid-crystalline ordering of antimicrobial peptide-DNA complexes controls TLR9 activation. Nature Materials 14, 696-700, (2015). This structural data showed that the dsDNA-gld-1 complex presents parallel DNA at an inter-DNA spacing that promotes binding of multiple TLR9s. Thus, gluten derived peptide gld-1 was found to form strongly immunomodulatory complexes with dsDNA.

### Gluten Peptide Fragments Induce Immune Activation

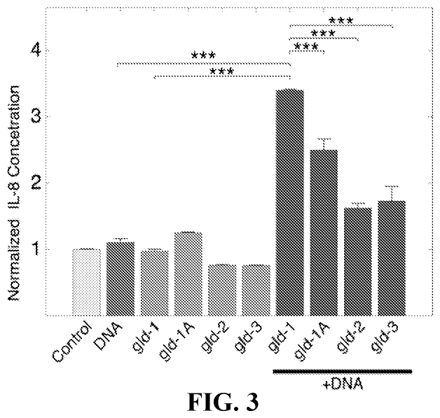

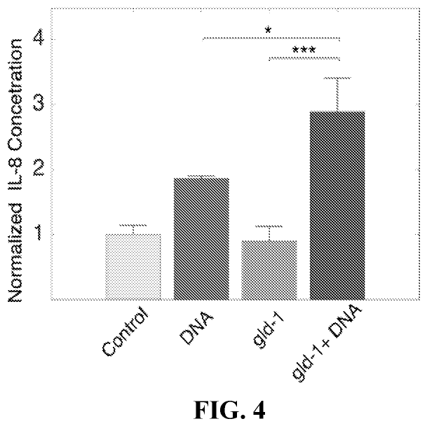

In order to test the biological impact of gluten peptide fragment/dsDNA complexes, human monocytes (THP-1) and human colorectal adenocarcinoma cells (HT-29) were treated with medium as a control, dsDNA alone, gld-1 alone, or dsDNA+gld-1 for 18 hours, then production and secretion of interleukin-8 (IL-8) were assessed by ELISA. When THP-1 cells were exposed to the dsDNA-gld-1 complex, a significant increase in IL-8 secretion was observed compared to the media control or to dsDNA or gld-1 treatment groups alone (FIG. 3). Similarly, HT-29 cells demonstrated an increased secretion of IL-8 when exposed to the dsDNA-gld-1 complex compared to dsDNA or gld-1 alone or media control (FIG. 4).

### Immune Activation is Mediated by a TLR-Specific Mechanism

To assess the activation of NF-κB/IL-8 in response to TLR9-specific recognition of the dsDNA-gld-1 complex, HEK293 cells (which do not express TLR9) were transfected with plasmids containing luciferase under the control of the IL-8 promoter and human wild-type TLR9, or a pcDNA3.1 vector as control, using Lipofectamine LTX (Life Technologies, Inc). After 48 hours of recovery, cells were then treated for 4 hours with medium as a control, dsDNA alone, gld-1 peptide alone, or dsDNA-gld-1 complex, and luciferase activity measured.

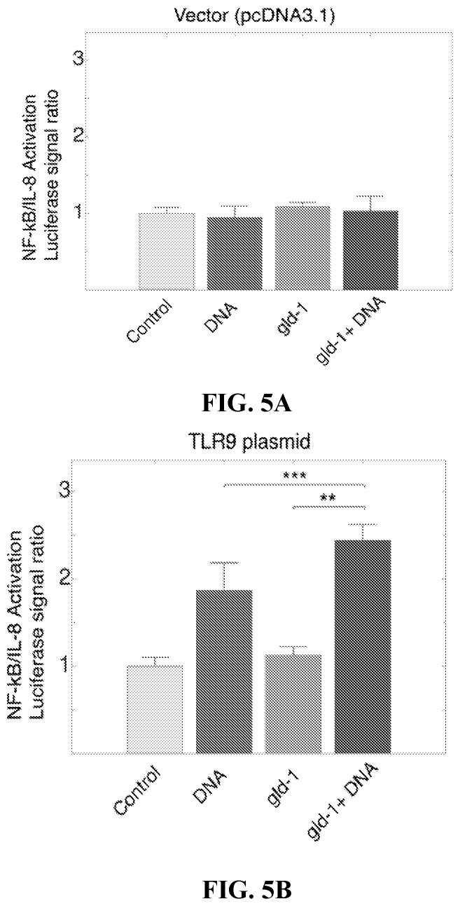

As shown in FIG. 5A, when exposed to dsDNA, gld-1, or the dsDNA-gld-1 complex, cells transfected with the vector control had comparable NF-κB/IL-8 luciferase reporter signal to the media control. In contrast, cells carrying the TLR9 plasmid presented with a significant increase in the levels of NF-κB/IL-8 activation for DNA and dsDNA-gld-1 complex compared to the media control; the gld-1 peptide alone did not significantly increase activation (FIG. 5B). The increase of activation in the presence of DNA in cells carrying the TLR9 plasmid, but not the vector, confirmed that the transfected TLR9 is functional in this approach. Strikingly, there was significantly higher activation by the dsDNA-gld-1 complex compared to dsDNA alone, which confirmed the critical role played by the gluten derived peptide gld-1.

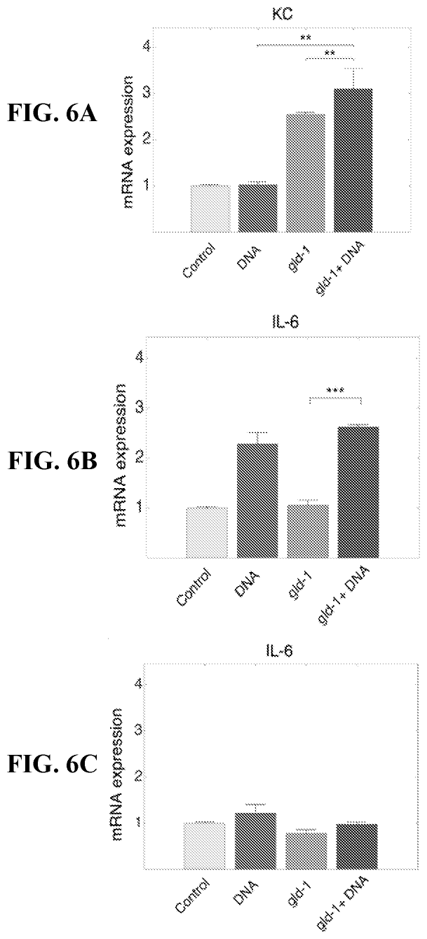

To further validate the role of the dsDNA-gld-1 complex in the innate immune activation of TLR9, TLR9 knock-out (TLR9KO) mouse macrophages were compared to wild-type controls. Similar to IL-8 release by THP-1 and HT-29 cells, wild-type macrophages showed a significant increase in cytokine gene expression (keratinocyte-derived chemokine, KC, and interleukin-6, IL-6) in the presence of the dsDNA-gld-1 complex (FIGS. 6A-B). Such increased gene expression was attenuated in TLR9KO macrophages, which lack TLR9 (FIG. 6C).

### Mutations of Gluten Peptide Fragments Disrupt Peptide-dsDNA Interaction, Resulting in Impaired TLR9 Presentation

To address the immune activity of the gld-1 peptide, mutations were strategically engineered to disrupt the dsDNA-gld-1 complex. Mutations were designed based on the physiochemical properties necessary for the proper formation of the correct nanocrystalline structures of peptide and dsDNA that can activate TLRs (such as in Lee, E. Y. et al. Helical antimicrobial peptides assemble into protofibril scaffolds that present ordered dsDNA to TLR9. Nature Communications 10, 1012 (2019) and Lee, E. Y., et al. Mapping membrane activity in undiscovered peptide sequence space using machine learning. Proceedings of the National Academy of Sciences 113, 13588-13593 (2016)). These properties included, but were not limited to, charged amino acid content, hydrophobicity, steric bulkiness, and dipole moment, all of which could impact the nanoscopic spatial periodicity of the nucleic acid. Four different candidate mutants were used to assess how alteration of these physiochemical properties that impact peptide-nucleic acid assembly could attenuate immune reaction.

One way to suppress immune activation is to reduce the peptide cationic charge to reduce peptide interaction with DNA. Therefore, single (gld-2) and double (gld-3) mutations were introduced to replace cationic amino acids with other amino acids in conditions prevalent for the gastrointestinal tract. For example, glutamine was used, although other amino acids with similar charge profiles could also be used. See, Table 2 for further details. In other mutants, cationic amino acids were replaced with anionic amino acids.

The ability of the gld-2 and gld-3 peptides to complex with dsDNA and to stimulate cytokine release in THP-1 monocytes was then tested.

Genomic E. coli dsDNA was incubated with gld-2 and gld-3 to test the formation of an ordered dsDNA-peptide complex. Association of gld-2 and gld-3 with dsDNA similar to gld-1 was observed (FIG. 8C). However, SAXS measurements revealed the inability of these two peptides to form an ordered nanocrystalline columnar structure with dsDNA, as demonstrated by the absence of scattering Bragg peaks in their respective dsDNA-peptide complex spectra (FIG. 8A). Hence, while the gld-2 and gld-3 associated with dsDNA, they lacked the ability to organize dsDNA at an inter-DNA spacing that promotes binding of multiple TLR9s to amplify an immune response.

Both gld-2 and gld-3 elicited an attenuated production of IL-8 in the presence of dsDNA compared to the dsDNA-gld-1 complex (FIG. 3). These results demonstrated that even single amino acid mutations at strategically selected sites disrupted the peptide-dsDNA interaction from gld-1 and reduced its immune activity.

Next, a variant of gld-1 in which all cysteines were replaced with alanines (gld-1A) was assessed to test the importance of these residues for peptide association with dsDNA. Disulfide bond formation can lead to multimerization and network formation. Interestingly, these mutations reduced the dsDNA-peptide complex activity compared to the original dsDNA-gld-1 complex (FIG. 3). This result suggested that the capacity to form disulfide bond networks may lead to increased innate immune activation. Disulfide bond formation is important for gluten network properties, such as in dough preparation. Therefore, while decreasing the ability to form disulfide bonds is one way to decrease inflammation by reducing effective steric bulk and impacting DNA organization in the complex, it also impacts the mechanical properties of gluten.

### Immunomodulatory Activity of Glutenin Peptide Fragments

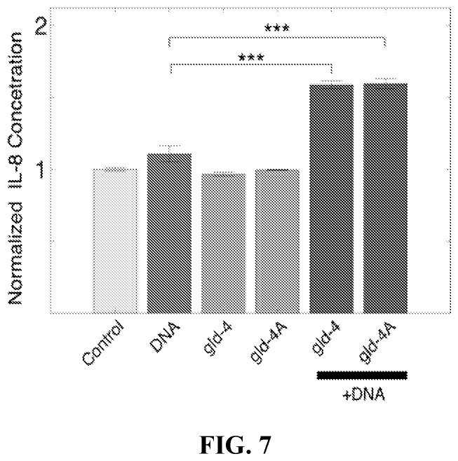

The equally important component in gluten, glutenin is present at about similar amounts to that of gliadin in the wheat kernel. Hence, the identified peptide gld-4 from Example 1, above, was tested for its ability to organize dsDNA and stimulate cytokine release in THP-1 monocytes. A cysteine-to-alanine mutant gld-4A (similar to gld-1A) was generated and tested to assess the importance of these cysteines on immune activation. Although gld-4+dsDNA stimulated IL-8 production compared to dsDNA or gld-4 alone, such increases were comparable to the increase seen with gld-2 and gld-3 with dsDNA, and was smaller than the dsDNA-gld-1 complex (FIG. 7). No significant difference was found between gld-4 and gld-4A. Therefore, these results further implicated gld-1 from α-gliadin as responsible for triggering large immune responses and thus an important peptide target for immunomodulation.

### A Generalized Immunomodulatory Gluten and Gluten-Related Peptide Sequence

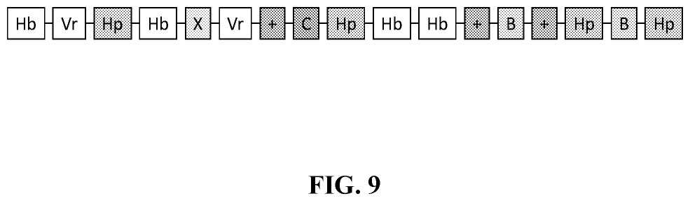

The results described above demonstrated a bioactive peptide in gluten to be primarily present in the non-repetitive region 1 (NRR1) of α-gliadin proteins (highly conserved among isoforms) and encompassing 23 amino acids in this region. The 19-mer peptide, referred to herein as gld-1, is not only present across other gliadin forms (e. g., β, γ, and ω gliadins) and glutenin, but also across different forms of gluten in other related cereal grains, i. e., barley, oats, and even corn. The corresponding general amino acid formula is shown in FIG. 9 and provided below (SEQ ID NO: 55):

Hb-Vr-Hp-Hb-X-Vr-+-C-Hp-Hb-Hb-+-B-+-Hp-B-Hp

The basic amino acid composition of the NRR1 region of interest that encompasses the 23 amino acids is referred to as the “complex forming gluten peptide” (CFGP). In the formula above, Hb is any hydrophobic amino acid [primarily leucine (L), isoleucine (I), valine (V), or alanine (A)]; Hp is any hydrophilic amino acid [primarily glutamine (Q), asparagine (N), serine (S), histidine (H), arginine (R), and in some cases A]; X is a 3-mer sequence containing two hydrophilic amino acids [primarily proline (P), Q, N, S] and an anionic amino acid [glutamate (E) or aspartate (D)]; B is a 3-mer sequence containing hydrophobic amino acids [primarily A, V, L, I, phenylalanine (F) or methionine (M)] and one hydrophilic amino acid [primarily glycine (G), threonine (T), N, S]; C stands for the amino acid cystine [in some cases replaced by H or tyrosine (Y)]; the symbol + represents primary locations of cationic amino acids, i.e., H, R, and lysine (K) [in some cases replaced by Q, I, L, Y, F, D, or E]; and Vr can be any amino acid.

The data discussed above showed that single mutations could disrupt the immune activity of the original consensus sequence gld-1, as shown in FIG. 3. The altered immunogenicity of mutated gld-1 peptides from the wild-type gld-1 peptide is caused by disruptions in the formation of ordered complexes between the wild-type gld-1 peptide fragment and nucleic acids (e.g., dsDNA), as a specific range of values for the inter-nucleic acid spacing is necessary for the identified immune activation. As a result, mutations that diminished the capacity for these organized complexes to assemble also decreased markers of immune activation. It was also demonstrated that reducing the positive charge of the CFGP, as well as affecting its steric bulk properties, could lead to an attenuation of CFGP-induced immune activity. Hence, below are mutations to CFGP that can reduce its immunomodulation capabilities:
Insertion of negatively charged amino acids (D or E) anywhere along the CFGP sequence.
Substitution with a negatively charged amino acid (D or E) at any position along the CFGP sequence.
Substitution of [+] or [Hp], if [Hp] is a positively charged amino acid (K, R, or H), with any amino acid.
Substitution of positively charged amino acid (K, R, or H) in [B] with any amino acid, if [B] contains any K, R, or H.
Deletion of [+] or [Hp] (if [Hp] is positively charged amino acid (K, R, or H)).
Deletion of positively charged amino acid (K, R, or H) in [B] if it contains K, R, or H.
Insertion of amino acids to CFGP.
Partial deletions to CFGP.
Deletion of [C].
Substitution of [C] to any amino acid.
Substitution of ‘bulky’ hydrophobic amino acids (F, Y, and W) for any amino acid.
Substitution of hydrophobic amino acids (A, L, I, V, M, Y, W, or F) to hydrophilic amino acids (S, T, N, Q, G, P, C, K, R, H, K, D, or E).
Substitution of hydrophilic amino acids (S, T, N, Q, G, P, C, K, R, H, K, D, or E) to hydrophobic amino acids (A, L, I, V, M, Y, W, or F).
Substitution of proline (P) in [X] with any amino acid, if [X] contains any P.
Deletion of proline (P) in [X] with any amino acid, if [X] contains any P.

TABLE 3
Sequences of the disclosure.

| SEQ ID | | |
|---|---|---|
| NO | SEQUENCE | DESCRIPTION |
| 1 | MKTFLILALLAIVATTATTAVRVPVPQ | A/B-GLIADIN A-I (TRITICUM |
| | LQPQNPSQQQPQEQVPLVQQQQFLGQ | AESTIVUM) |
| | QQPFPPQQPYPQPQPFPSQQPYLQLQPF | |
| | LQPQLPYSQPQPFRPQQPYPQPQPQYS | |
| | QPQQPISQQQQQQQQQQQQQQQQQQ | |
| | QIIQQILQQQLIPCMDVVLQQHNIVHG | |
| | KSQVLQQSTYQLLQELCCQHLWQIPEQ | |
| | SQCQAIHNVVHAIILHQQQKQQQQPSS | |
| | QVSFQQPLQQYPLGQGSFRPSQQNPQA | |
| | QGSVQPQQLPQFEEIRNLARK | |
| 2 | MKTFPILALLAIVATTATTAVRVPVPQ | A/B-GLIADIN A-II |
| | LQLQNPSQQQPQEQVPLVQEQQFQGQ | (TRITICUM AESTIVUM) |
| | QQPFPPQQPYPQPQPFPSQQPYLQLQPF | |
| | PQPQLPYPQPQPFRPQQPYPQPQPQYS | |
| | QPQQPISQQQQQQQQQQQQQQQILQQI | |
| | LQQQLIPCRDVVLQQHNIAHGSSQVLQ | |
| | ESTYQLVQQLCCQQLWQIPEQSRCQAI | |
| | HNVVHAIILHQQHHHHQQQQQQQQQ | |
| | QPLSQVSFQQPQQQYPSGQGFFQPSQQ | |
| | NPQAQGSFQPQQLPQFEEIRNLALQTLP | |
| | AMCNVYIPPYCTIAPFGIFGTN | |
| 3 | MKTFLILALLAIVATTATSAVRVPVPQ | A/B-GLIADIN A-III |
| | LQPQNPSQQQPQEQVPLMQQQQQFPG | (TRITICUM AESTIVUM) |
| | QQEQFPPQQPYPHQQPFPSQQPYPQPQ | |
| | PFPPQLPYPQTQPFPPQQPYPQPQPQYP | |
| | QPQQPISQQQAQQQQQQQQTLQQILQ | |
| | QQLIPCRDVVLQQHNIAHASSQVLQQS | |
| | SYQQLQQLCCQQLFQIPEQSRCQAIHN | |
| | VVHAIILHHHQQQQQQPSSQVSYQQPQ | |
| | EQYPSGQVSFQSSQQNPQAQGSVQPQ | |
| | QLPQFQEIRNLALQTLPAMCNVYIPPY | |
| | CSTTIAPFGIFGTN | |
| 4 | MKTFLILALRAIVATTATIAVRVPVPQL | A/B-GLIADIN A-IV |
| | QPQNPSQQQPQKQVPLVQQQQFPGQQ | (TRITICUM AESTIVUM) |
| | QPFPPQQPYPQQQPFPSQQPYMQLQPF | |
| | PQPQLPYPQPQLPYPQPQPFRPQQSYPQ | |
| | PQPQYSQPQQPISQQQQQQQQQQQQQ | |
| | QQILQQILQQQLIPCRDVVLQQHSIAHG | |
| | SSQVLQQSTYQLVQQFCCQQLWQIPEQ | |
| | SRCQAIHNVVHAIILHQQQQQQQQQQ | |
| | QQQQQPLSQVCFQQSQQQYPSGQGSF | |
| | QPSQQNPQAQGSVQPQQLPQFEEIRNL | |
| | ALETLPAMCNVYIPPYCTIAPVGIFGTN | |
| 5 | MKTFLILALLAIVATTATTAVRVPVPQ | A/B-GLIADIN A-V |
| | LQPQNPSQQQPQEQVPLVQQQQFPGQ | (TRITICUM AESTIVUM) |
| | QQQFPPQQPYPQPQPFPSQQPYLQLQPF | |
| | PQPQPFPPQLPYPQPQSFPPQQPYPQQQ | |
| | PQYLQPQQPISQQQAQQQQQQQQQQQ | |
| | QQQQILQQILQQQLIPCRDVVLQQHNI | |
| | AHASSQVLQQSTYQLLQQLCCQQLLQI | |
| | PEQSQCQAIHNVAHAIIMHQQQQQQQ | |
| | EQKQQLQQQQQQQQQLQQQQQQQQQ | |
| | QPSSQVSFQQPQQQYPSSQVSFQPSQL | |
| | NPQAQGSVQPQQLPQFAEIRNLALQTL | |
| | PAMCNVYIPPHCSTTIAPFGISGTN | |
| 6 | MAKRLVLFAAVVIALVALTTAEGEAS | GLUTENIN, HIGH |
| | RQLQCERELQESSLEACRQVVDQQLA | MOLECULAR WEIGHT |
| | GRLPWSTGLQMRCCQQLRDVSAKCRS | SUBUNIT 12 (TRITICUM |
| | VAVSQVARQYEQTVVPPKGGSFYPGE | AESTIVUM) |
| | TTPLQQLQQGIFWGTSSQTVQGYYPSV | |
| | TSPRQGSYYPGQASPQQPGQGQQPGK | |
| | WQEPGQGQQWYYPTSLQQPGQGQQIG | |
| | KGKQGYYPTSLQQPGQGQQIGQGQQG | |
| | YYPTSPQHTGQRQQPVQGQQIGQGQQ | |
| | PEQGQQPGQWQQGYYPTSPQQLGQGQ | |
| | QPGQWQQSGQGQQGHYPTSLQQPGQ | |
| | GQQGHYLASQQQPAQGQQGHYPASQ | |
| | QQPGQGQQGHYPASQQQPGQGQQGH | |
| | YPASQQEPGQGQQGQIPASQQQPGQG | |
| | QQGHYPASLQQPGQQGHYPTSLQQLG | |
| | QGQQIGQPGQKQQPGQGQQTGQGQQP | |
| | EQEQQPGQGQQGYYPTSLQQPGQGQQ | |
| | QGQGQQGYYPTSLQQPGQGQQGHYP | |
| | ASLQQPGQGQGQPGQRQQPGQGQHPE | |
| | QGQQPGQGQQGYYPTSPQQPGQGQQL | |
| | GQGQQGYYPTSPQQPGQGQQPGQGQQ | |
| | GHCPMSPQQTGQAQQLGQGQQIGQVQ | |
| | QPGQGQQGYYPTSLQQPGQGQQSGQG | |
| | QQSGQGHQPGQGQQSGQEKQGYDSPY | |
| | HVSAEQQAASPMVAKAQQPATQLPTV | |
| | CRMEGGDALSASQ | |
| 7 | HGKSQVLQQSTYQLLQELCCQHLWQI | A/B-GLIADIN A-I (TRITICUM |
| | PEQSQCQAIHNVVHAIILHQQQ | AESTIVUM) ISG |
| 8 | HGSSQVLQESTYQLVQQLCCQQLWQI | A/B-GLIADIN A-II |
| | PEQSRCQAIHNVVHAIILHQQHHHHQQ | (TRITICUM AESTIVUM) ISG |
| 9 | HASSQVLQQSSYQQLQQLCCQQLFQIP | A/B-GLIADIN A-III |
| | EQSRCQAIHNVVHAIILHHHQ | (TRITICUM AESTIVUM) ISG |
| 10 | VLQQSTYQLVQQFCCQQLWQIPEQSR | A/B-GLIADIN A-IV |
| | CQAIHNVVHAIILHQQQQQQQ | (TRITICUM AESTIVUM) ISG |
| 11 | HASSQVLQQSTYQLLQQLCCQQLLQIP | A/B-GLIADIN A-V |
| | EQSQCQAIHNVAHAIIMHQQQQQQQE | (TRITICUM AESTIVUM) ISG |
| | QKQQLQQ | |
| 12 | QVVDQQLAGRLPWSTGLQMRCCQQL | GLUTENIN, HIGH |
| | RDVSAKCRSVAVSQVAR | MOLECULAR WEIGHT |
| | | SUBUNIT 12 (TRITICUM |
| | | AESTIVUM) ISG |
| 13 | MKILIILTILAMATTFATSEMQVNPSVQ | Γ-HORDEIN-1 POLYPEPTIDE |
| | VQPTQQQPYPESQQPFISQSQQQFPQPQ | FROM BARLEY UNIPROT |
| | QPFPQQPQQPFPQSQQQCLQQPQHQFP | ENTRY P17990 |
| | QPTQQFPQRPLLPFTHPFLTFPDQLLPQ | |
| | PPHQSFPQPPQSYPQPPLQPFPQPPQQK | |
| | YPEQPQQPFPWQQPTIQLYLQQQLNPC | |
| | KEFLLQQCRPVSLLSYIWSKIVQQSSCR | |
| | VMQQQCCLQLAQIPEQYKCTAIDSIVH | |
| | AIFMQQGQRQGVQIVQQQPQPQQVGQ | |
| | CVLVQGQGVVQPQQLAQMEAIRTLVL | |
| | QSVPSMCNFNVPPNCSTIKAPFVGVVT | |
| | GVGGQ | |
| 14 | ITTTTMQFNPSGLELERPQQLFPQWQP | Γ-HORDEIN-3 POLYPEPTIDE |
| | LPQQPPFLQQEPEQPYPQQQPLPQQQPF | FROM BARLEY UNIPROT |
| | PQQPQLPHQHQFPQQLPQQQFPQQMPL | ENTRY P80198 |
| | QPQQQFPQQMPLQPQQQPQFPQQKPF | |
| | GQYQQPLTQQPYPQQQPLAQQQPSIEE | |
| | QHQLNLCKEFLLQQCTLDEKVPLLQSV | |
| | ISFLRPHISQQNSCQLKRQQCCQQLANI | |
| | NEQSRCPAIQTIVHAIVMQQQVQQQVG | |
| | HGFVQSQLQQLGQGMPIQLQQQPGQA | |
| | FVLPQQQAQFKVVGSLVIQTLPMLCNV | |
| | HVPPYCSPFGSMATGSGGQ | |
| 15 | MKTFLIFALLAIAATSTIAQQQPFPQQPI | B1-HORDEIN POLYPEPTIDE |
| | PQQPQPYPQQPQPYPQQPFPPQQPFPQ | FROM BARLEY UNIPROT |
| | QPVPQQPQPYPQQPFPPQQPFPQQPPF | ENTRY P06470 |
| | WQQKPFPQQPPFGLQQPILSQQQPCTP | |
| | QQTPLPQGQLYQTLLQLQIQYVHPSIL | |
| | QQLNPCKVFLQQQCSPVPVPQRIARSQ | |
| | MLQQSSCHVLQQQCCQQLPQIPEQFRH | |
| | EAIRAIVYSIFLQEQPQQLVEGVSQPQQ | |
| | QLWPQQVGQCSFQQPQPQQVGQQQQ | |
| | VPQSAFLQPHQIAQLEATTSIALRTLPM | |
| | MCSVNVPLYRILRGVGPSVGV | |
| 16 | MKTLLMLAILAMATTIATANMQVNPS | 75K GAMMA SECALIN |
| | GQVQRPQQQPFPQPQQSSPQQPQQPFP | POLYPEPTIDE FROM RYE |
| | QQPQQPVPQQSQQSFPQTQQPQQPLPQ | UNIPROT ENTRY H6ULI8 |
| | PQQPQQLFPQPQQSSPQQSQQVTSQPQ | |
| | QPFPQAQPPQQSCPQSQQPYPQEPQQL | |
| | FPQSQQPQQPFPQPQQPQQPFPQPQPQT | |
| | QQSIPQPQQPFPQYQEPFPQVHQPQQPS | |
| | PQQQQPSIQLSLQQQLNPCKNVLLQQC | |
| | SPVALVSSLRSKIFPQSECQVMQQQCC | |
| | QQLAQIPQQLQCAAIHSVVHAIIMQQE | |
| | QREGVQILLPQSHQQHVGQGALAQVQ | |
| | GIIQPQQLSQLEVVRSLVLQNLPTMCNI | |
| | YVPRQCSTIQAPFASIVTGIVGH | |
| 17 | MKTFLIFALLAMAATMATAQFDPSEQ | AVENIN-3 POLYPEPTIDE |
| | YQPYPEQQQPILQQQQMLLQQQQQML | FROM OAT UNIPROT ENTRY |
| | LQQQPLLQVLQQQLNPCRQFLVQQCSP | P80356 |
| | VAVVPFLRSQILQQSSCQVMRQQCCR | |
| | QLEQIPEQLRCPAIHSVVQAIIMQQQQF | |
| | FQPQMQQQFFQPQMQQVTQGIFQPQM | |
| | QQVTQGIFQPQLQQVTQGIFQPQMQG | |
| | QIEGMRAFALQALPAMCDVYVPPHCP | |
| | VATAPLGGF | |
| 18 | TTTVQYNPSEQYQPYPEQQEPFVQQQP | AVENIN-E POLYPEPTIDE |
| | FVQQQQQPFVQQQQMFLQPLLQQQLN | FROM OAT UNIPROT ENTRY |
| | PCKQFLVQQCSPVAVVPFLRSQILRQAI | Q09114 |
| | CQVARQQCCRQLAQIPEQLRCPAIHSV | |
| | VQAIILQQQQQQQFFQPQLQQQVFQPQ | |
| | LQQVFNQPQQQAQFEGMRAFALQALP | |
| | AMCDVYVPPQCPVATAPLGGF | |
| 19 | LARSQMWQQSSCHVMQQQCCQQLQQ | Γ-GLIADIN B-I (TRITICUM |
| | IPEQSRYEAIRAIIYSIILQEQQQGFVQP | AESTIVUM) ISG |
| | Q | |
| 20 | SLWSIIWPQSDCQVMRQQCCQQLAQIP | Γ-GLIADIN B-II (TRITICUM |
| | QQLQCAAIHSVVHSIIMQQQQQQQQQ | AESTIVUM) ISG |
| | QGID | |
| 21 | LARSQMLQQSSCHVMQQQCCQQLPQI | Γ-GLIADIN B-III (TRITICUM |
| | PQQSRYQAIRAIIYSIILQEQQQVQGSIQ | AESTIVUM) ISG |
| | SQ | |
| 22 | IARSQMLQQSSCHVLQQQCCQQLPQIP | B1-HORDEIN (HORDEUM |
| | EQFRHEAIRAIVYSIFLQEQPQ | VULGARE) ISG |
| 23 | IARSQMLQQSSCHVLQQQCCQQLPQIP | B3-HORDEIN (HORDEUM |
| | EQLRHEAVRAIVYSIVLQEQSL | VUGARE) ISG |
| 24 | YIWSKIVQQSSCRVMQQQCCLQLAQIP | Γ-HORDEIN-1 (HORDEUM |
| | EQYKCTAIDSIVHAIFMQQGQRQGVQI | VUGARE) ISG |
| 25 | FLRPHISQQNSCQLKRQQCCQQLANIN | Γ-HORDEIN-3 (HORDEUM |
| | EQSRCPAIQTIVHAIVMQQQVQQ | VUGARE) ISG |
| 26 | FLRSQILRQAICQVARQQCCRQLAQIPE | AVENIN-E (AVENA SATIVA) |
| | QLRCPAIHSVVQAIILQQQQQQQFFQP | ISG |
| | QLQQQ | |
| 27 | FLRSQILQQSSCQVMRQQCCRQLEQIP | AVENIN-3 (AVENA SATIVA) |
| | EQLRCPAIHSVVQAIIMQQQQFFQPQM | ISG |
| | QQQ | |
| 28 | ILQQILQQQLIPCRDVVLQQHNIAHGSS | CONS-AGLD-NRR1 |
| | QVLQQSTYQLLQQLCCQQLWQIPEQS | |
| | RCQAIHNVVHAIILHQQQQQ | |
| 29 | LWQIPEQSRCQAIHNVVHA | GLD-1 |
| 30 | LAAQLWQIPEQSRAQAIHNVVH | GLD-1A |
| 31 | LWQIPEQSQCQAIHNVVHA | GLD-2 |
| 32 | LWQIPEQSQCQAIHNVVQA | GLD-3 |
| 33 | RCCQQLRDVSAKCRSVAVSQVAR | GLD-4 |
| 34 | RAAQQLRDVSAKARSVAVSQVAR | GLD-4A |
| 35 | VQQLCCQQLWQIPEQSRCQAIHNVVH | MM1 |
| | AIILHQQQQQ | |
| 36 | LQELCCQHLWQIPEQSQCQAIHNVVH | A-I |
| | AIILHQQQK | |
| 37 | VQQLCCQQLWQIPEQSRCQAIHNVVH | A-II |
| | AIILHQQHHHHQQ | |
| 38 | LQQLCCQQLFQIPEQSRCQAIHNVVHA | A-III |
| | IILHHHQQ | |
| 39 | VQQFCCQQLWQIPEQSRCQAIHNVVH | A-IV |
| | AIILHQQQQQQQQ | |
| 40 | LQQLCCQQLLQIPEQSQCQAIHNVAHA | A-V |
| | IIMHQQQQQQQEQ | |
| 41 | LQQLCCQQLWQIPEQSRCQAIHNVVH | PW1215 |
| | AIILHQQQR | |
| 42 | LQQLCCQQLLQIPEQSRCQAIHNVVHA | PW8142 |
| | IIMHQQEQQQQLQ | |
| 43 | LQELCCQHLWQIPEQSQCQAIHNVVH | PTO-A10 |
| | AIILHQQQQK | |
| 44 | LQQLCCQQLWQIPEQSRCQAIHNVVH | CONSENSUS |
| | AIILH | |
| 45 | MRVLLVALALLALAASATSTHTSGGC | GLUTELIN-2 |
| | GCQPPPPVHLPPPVHLPPPVHLPPPVHL | |
| | PPPVHLPPPVHLPPPVHVPPPVHLPPPP | |
| | CHYPTQPPRPQPHPQPHPCPCQQPHPSP | |
| | CQLQGTCGVGSTPILGQCVEFLRHQCS | |
| | PTPQPHPQPHPCPCQQPHPSPCQLQGTC | |
| | GVGSTPILGQCVEFLRHQCSPTATPYCS | |
| | PQCQSLRQQCCQQLRQVEPQHRYQAIF | |
| | GLVLQSILQQQPQSGQVAGLLAAQIAQ | |
| | QLTAMCGLQQPTPCPYAAAGGVPH | |
| 46 | MKLVLVVLAFIALVSSVSCTQTGGCSC | 50-KDA-GAMMA-ZEIN |
| | GQQQSHEQQHHPQQHHPQKQQHQPPP | |
| | QHHQQQQHQQQQVHMQPQKHQQQQ | |
| | EVHVQQQQQQPQHQQQQQQQQHQQQ | |
| | HQCEGQQQHHQQSQGHVQQHEQSHE | |
| | QHQGQSHEQQHQQQFQGHDKQQQPQ | |
| | QPQQYQQGQEKSQQQQCHCQEQQQT | |
| | TRCSYNYYSSSSNLKNCHEFLRQQCSP | |
| | LVMPFLQSRLIQPSSCQVLQQQCCHDL | |
| | RQIEPQYIHQAIYNMVQSIIQEEQQQQP | |
| | CELCGSQQATQSAVAILTAAQYLPSMC | |
| | GLYHSYYQNNPCSSNDISGVCN | |
| 47 | QQPVSRQPQQIIPQQPQQPFPLQPQQPQ | B3-HORDEIN |
| | PFPQQPIPQQPQPYPQQPQSFPQQPFPS | |
| | QQPFPQQPPFWQQQPVLSQQQPCTQD | |
| | QTPLLQEQQDQMLVQVQIPFVHPSILQ | |
| | QLNPCKVFLQQQCSPLAMSQRIARSQ | |
| | MLQQSSCHVLQQQCCQQLPQIPEQLRH | |
| | EAVRAIVYSIVLQEQSLQLVQGVSQPQ | |
| | QQSQQQQVGQCSFQQPQPQQGQQQQ | |
| | VPQSVFLQPHQIAQLEATTSIALRTLPT | |
| | MCSVNVPLYRIVPLAIDTRVGV | |
| 48 | MKTFLIFALLAIAATSTIAQQQPFPQQPI | B1-HORDEIN |
| | PQQPQPYPQQPQPYPQQPFPPQQPFPQ | |
| | QPVPQQPQPYPQQPFPPQQPFPQQPPF | |
| | WQQKPFPQQPPFGLQQPILSQQQPCTP | |
| | QQTPLPQGQLYQTLLQLQIQYVHPSIL | |
| | QQLNPCKVFLQQQCSPVPVPQRIARSQ | |
| | MLQQSSCHVLQQQCCQQLPQIPEQFRH | |
| | EAIRAIVYSIFLQEQPQQLVEGVSQPQQ | |
| | QLWPQQVGQCSFQQPQPQQVGQQQQ | |
| | VPQSAFLQPHQIAQLEATTSIALRTLPM | |
| | MCSVNVPLYRILRGVGPSVGV | |
| 49 | MKTFLILALLAIVATTARIAVRVPVPQL | ALPHA-GLIADIN |
| | QPQNPSQQQPQEQVPLVQQQQFPGQQ | |
| | QPFPPQQPYPQPQPFPSQQPYLQLQPFP | |
| | QPQLPYPQPQLPYPQPQLPYPQPQPFRP | |
| | QQPYPQSQPQYSQPQQPISQQQQQQQQ | |
| | QQQQKQQQQQQQQILQQILQQQLIPCR | |
| | DVVLQQHSIAYGSSQVLQQSTYQLVQ | |
| | QLCCQQLWQIPEQSRCQAIHNVVHAII | |
| | LHQQQQQQQQQQQQPLSQVSFQQPQQ | |
| | QYPSGQGSFQPSQQNPQAQGSVQPQQ | |
| | LPQFEEIRNLALETLPAMCNVYIPPYCT | |
| | IAPVGIFGTN | |
| 50 | ITTTTMQFNPSGLELERPQQLFPQWQP | GAMMA-HORDEIN-3 |
| | LPQQPPFLQQEPEQPYPQQQPLPQQQPF | |
| | PQQPQLPHQHQFPQQLPQQQFPQQMPL | |
| | QPQQQFPQQMPLQPQQQPQFPQQKPF | |
| | GQYQQPLTQQPYPQQQPLAQQQPSIEE | |
| | QHQLNLCKEFLLQQCTLDEKVPLLQSV | |
| | ISFLRPHISQQNSCQLKRQQCCQQLANI | |
| | NEQSRCPAIQTIVHAIVMQQQVQQQVG | |
| | HGFVQSQLQQLGQGMPIQLQQQPGQA | |
| | FVLPQQQAQFKVVGSLVIQTLPMLCNV | |
| | HVPPYCSPFGSMATGSGGQ | |
| 51 | MKILIILTILAMATTFATSEMQVNPSVQ | GAMMA-HORDEIN-1 |
| | VQPTQQQPYPESQQPFISQSQQQFPQPQ | |
| | QPFPQQPQQPFPQSQQQCLQQPQHQFP | |
| | QPTQQFPQRPLLPFTHPFLTFPDQLLPQ | |
| | PPHQSFPQPPQSYPQPPLQPFPQPPQQK | |
| | YPEQPQQPFPWQQPTIQLYLQQQLNPC | |
| | KEFLLQQCRPVSLLSYIWSKIVQQSSCR | |
| | VMQQQCCLQLAQIPEQYKCTAIDSIVH | |
| | AIFMQQGQRQGVQIVQQQPQPQQVGQ | |
| | CVLVQGQGVVQPQQLAQMEAIRTLVL | |
| | QSVPSMCNFNVPPNCSTIKAPFVGVVT | |
| | GVGGQ | |
| 52 | IXGRX | Ile-(Glu or Asp)-Gly-Arg-X |
| 53 | LVPRGS | Leu-Val-Pro-Arg↓Gly-Ser |
| 54 | DDDDKX | Asp-Asp-Asp-Asp-Lys-X |
| 55 | Hb-Vr-Hp-Hb-X-Vr-+-C-Hp-Hb-Hb-+- | Complex forming gluten peptide as |
| | B-+-Hp-B-Hp | shown in FIG. 9. |
| | | Hb is any hydrophobic amino acid |
| | | [primarily leucine (L), isoleucine (I), |
| | | valine (V), or alanine (A)]; Hp is any |
| | | hydrophilic amino acid [primarily |
| | | glutamine (Q), asparagine (N), |
| | | serine (S), histidine (H), arginine |
| | | (R), and in some cases A]; X is a 3- |
| | | mer sequence containing two |
| | | hydrophilic amino acids [primarily |
| | | proline (P), Q, N, S] and an anionic |
| | | amino acid [glutamate (E) or |
| | | aspartate (D)]; B is a 3-mer sequence |
| | | containing hydrophobic amino acids |
| | | [primarily A, V, L, I, phenylalanine |
| | | (F) or methionine (M)] and one |
| | | hydrophilic amino acid [primarily |
| | | glycine (G), threonine (T), N, S]; C |
| | | stands for the amino acid cystine [in |
| | | some cases replaced by H or tyrosine |
| | | (Y)]; the symbol + represents |
| | | primary locations of cationic amino |
| | | acids, i.e., H, R, and lysine (K) [in |
| | | some cases replaced by Q, I, L, Y, F, |
| | | D, or E]; and Vr can be any amino |
| | | acid. |

What is claimed is:

1. A method for producing a gluten polypeptide with a reduced inflammatory potential, the method comprising:
(a) providing an amino acid sequence of a gluten polypeptide;
(b) selecting an inflammatory amino acid sequence in gluten (ISG) in the amino acid sequence of the gluten polypeptide;
(c) introducing one or more alterations in the ISG, wherein the one or more alterations are selected from the group consisting of:
(i) a substitution of one or more amino acid residues,
(ii) a deletion of one or more amino acid residues,
(iii) an insertion of one or more amino acid residues,
and any combination thereof,
thereby generating a modified gluten polypeptide amino acid sequence; and
(d) producing a gluten polypeptide comprising the modified gluten polypeptide amino acid sequence generated in step (c),
thereby producing a gluten polypeptide with a reduced inflammatory potential.

2. A method for producing a modified plant, plant tissue, plant organ, plant part, or plant cell that comprises a gluten polypeptide with a reduced inflammatory potential, the method comprising introducing a nucleic acid molecule encoding a gluten polypeptide with a reduced inflammatory potential into the plant, plant tissue, plant organ, plant part, or plant cell, wherein the gluten polypeptide with a reduced inflammatory potential comprises an amino acid sequence comprising one or more alterations in an ISG relative to a corresponding wild type gluten polypeptide, wherein the one or more alterations are selected from the group consisting of:
(i) a substitution of one or more amino acid residues,
(ii) a deletion of one or more amino acid residues,
(iii) an insertion of one or more amino acid residues,
and any combination thereof,
thereby producing a modified plant, plant tissue, plant organ, plant part, or plant cell comprising a gluten polypeptide with a reduced inflammatory potential.

3. The method of claim 2, further comprising reducing the expression of one or more endogenous gluten polypeptides in the plant, plant tissue, plant organ, plant part, or plant cell.

4. The method of claim 2, further comprising introducing one or more alterations in one or more endogenous gluten polypeptides in the plant, plant tissue, plant organ, plant part, or plant cell.

5. The method of claim 4, wherein the one or more alterations comprise a deletion of a gene encoding the one or more endogenous gluten polypeptides, or a part thereof, in the plant, plant tissue, plant organ, plant part, or plant cell.

6. The method of any one of claims 2-5, wherein the plant, plant tissue, plant organ, plant part, or plant cell is a wheat, a rye, a barley or an oat plant, plant tissue, plant organ, plant part, or plant cell.

7. The method of any one of claims 1-6, wherein the gluten polypeptide is a gluten polypeptide from wheat, rye, barley or oat.

8. The method of any one of claims 1-7, wherein the gluten polypeptide is a gliadin polypeptide, a glutenin polypeptide, a hordein polypeptide, a secalin polypeptide, or an avenin polypeptide.

9. The method of any one of claims 1-8, wherein the ISG is present in a non-repetitive region 1 (NRR1) of the gluten polypeptide.

10. The method of any one of claims 1-9, wherein the ISG comprises the amino acid sequence Hb-Vr-Hp-Hb-X-Vr-+-C-Hp-Hb-Hb-+-B-+-Hp-B-Hp (SEQ ID NO: 55), wherein:
“Hb” is a hydrophobic amino acid, optionally wherein the hydrophobic amino acid is selected from the group consisting of leucine (L), isoleucine (I), valine (V), and alanine (A);
“Hp” is a hydrophilic amino acid, optionally wherein the hydrophilic amino acid is selected from the group consisting of glutamine (Q), asparagine (N), serine (S), histidine (H), arginine (R), and A;
“X” is a 3-mer sequence comprising two hydrophilic amino acids and an anionic amino acid, optionally wherein the hydrophilic amino acids are selected from the group consisting of proline (P), Q, N, and S, and the anionic amino acid is glutamate (E) or aspartate (D);
“B” is a 3-mer sequence comprising hydrophobic amino acids and one hydrophilic amino acid, optionally wherein the hydrophobic amino acids are selected from the group consisting of A, V, L, I, phenylalanine (F), and methionine (M), and the hydrophilic amino acid is selected from the group consisting of glycine (G), threonine (T), N, and S;
“C” is cysteine, H or tyrosine (Y);
“+” is an amino acid selected from the group consisting of H, R, lysine (K), Q, I, L, Y, F, D, and E; and
“Vr” is any amino acid.

11. The method of claim 10, wherein the gluten polypeptide is a gliadin polypeptide, a glutenin polypeptide, or a gluten or gluten-related polypeptide from a cereal grain, optionally wherein the cereal grain is barley, oat, or corn.

12. The method of claim 10 or claim 11, wherein the ISG comprises the amino acid sequence
(SEQ ID NO: 28)
ILQQILQQQLIPCRDVVLQQHNIAHGSSQVLQQSTYQLLQQLCCQQLWQ
IPEQSRCQAIHNVVHAIILHQQQQQ.

13. The method of claim 10 or claim 11, wherein the ISG comprises the amino acid sequence
(SEQ ID NO: 29)
LWQIPEQSRCQAIHNVVHA.

14. The method of claim 12 or claim 13, wherein the gluten polypeptide is a gliadin polypeptide, optionally wherein the gliadin polypeptide is an α-, β-, γ-, or ω-gliadin polypeptide.

15. The method of claim 10 or claim 11, wherein the ISG comprises the amino acid sequence
(SEQ ID NO: 33)
RCCQQLRDVSAKCRSVAVSQVAR.

16. The method of claim 15, wherein the gluten polypeptide is a glutenin polypeptide, optionally wherein the glutenin polypeptide is a high molecular weight glutenin polypeptide.

17. The method of any one of claims 10-16, wherein the one or more alterations in the ISG comprise one or more of:
an insertion of one or more negatively charged amino acids in the sequence of the ISG, optionally wherein the negatively charged amino acids are D or E;
a substitution of one or more amino acid residues in the ISG sequence with a negatively charged amino acid, optionally wherein the negatively charged amino acid is D or E;
a substitution of “+”, and/or “Hp” if “Hp” is a positively charged amino acid, with any amino acid residue, optionally wherein the positively charged amino acid residue is K, R, or H;
a substitution of one or more positively charged amino acids at position “B” with any amino acid if “B” comprises any of K, R, or H;
a deletion of “+”, and/or “Hp” if “Hp” is a positively charged amino acid, optionally wherein the positively charged amino acid K, R, or H;
a deletion of one or more positively charged amino acids at position “B” if “B” comprises any of K, R, or H;
an insertion of one or more amino acids;
a deletion of one or more amino acids;
a deletion of position “C”;
a substitution of position “C” with any amino acid;
a substitution of one or more bulky hydrophobic amino acids with any amino acid, optionally wherein the bulky hydrophobic amino acids are F, Y, or W;
a substitution of one or more hydrophobic amino acids to a hydrophilic amino acid, optionally wherein the hydrophilic amino acid is selected from the group consisting of S, T, N, Q, G, P, C, K, R, H, K, D, and E, and the hydrophobic amino acids are selected from the group consisting of A, L, I, V, M, Y, W, or F;
a substitution of one or more hydrophilic amino acids to a hydrophobic amino acid, optionally wherein the hydrophobic amino acid is selected from the group consisting of A, L, I, V, M, Y, W, and F, and the hydrophilic amino acids are selected from the group consisting of S, T, N, Q, G, P, C, K, R, H, K, D, and E;
a substitution of a proline amino acid residue in “X” with any amino acid if “X” contains a proline; and/or
a deletion of proline (P) in “X” with any amino acid, if “X” contains a proline.

18. The method of any one of claims 13-14 and 17, wherein the gluten polypeptide with a reduced inflammatory potential comprises a modified ISG comprising the sequence
(SEQ ID NO: 30)
LAAQLWQIPEQSRAQAIHNVVH,
(SEQ ID NO: 31)
LWQIPEQSQCQAIHNVVHA,
or
(SEQ ID NO: 32)
LWQIPEQSQCQAIHNVVQA.

19. The method of any one of claims 15-17, wherein the gluten polypeptide with a reduced inflammatory potential comprises a modified ISG comprising the sequence
(SEQ ID NO: 34)
RAAQQLRDVSAKARSVAVSQVAR.

20. The method of any one of claims 1-17, wherein the one or more alterations in an ISG comprise a substitution of one or more amino acid residues in the ISG sequence to the corresponding amino acid residue in a corresponding polypeptide from corn.

21. The method of any one of claims 1-17, wherein the one or more alterations in an ISG comprise a substitution of one or more amino acid residues in the ISG sequence to the corresponding amino acid residue in a corresponding gluten or gluten-related polypeptide from corn.

22. The method of claim 8, wherein the gliadin polypeptide is an α/β-gliadin polypeptide.

23. The method of claim 22, wherein the α/β-gliadin polypeptide is an α/β-gliadin A-I polypeptide.

24. The method of claim 23, wherein the α/β-gliadin A-I polypeptide comprises the amino acid sequence of SEQ ID NO: 1, or an amino acid sequence having at least about 85%, at least about 90%, at least about 95%, at least about 96%, at least about 97%, at least about 98%, at least about 99%, or 100% sequence identity to the amino acid sequence of SEQ ID NO: 1.

25. The method of claim 23 or claim 24, wherein the ISG comprises the amino acid sequence of amino acid residues 159-206 of SEQ ID NO: 1, or of amino acid residues corresponding to amino acid residues 159-206 of an α/β-gliadin A-I polypeptide.

26. The method of claim 23 or claim 24, wherein the ISG comprises the amino acid sequence of SEQ ID NO: 7, or an amino acid sequence having at least about 85%, at least about 90%, at least about 95%, at least about 96%, at least about 97%, at least about 98%, at least about 99%, or 100% sequence identity to the amino acid sequence of SEQ ID NO: 7.

27. The method of claim 22, wherein the α/β-gliadin polypeptide is an α/β-gliadin A-II polypeptide.

28. The method of claim 27, wherein the α/β-gliadin A-II polypeptide comprises the amino acid sequence of SEQ ID NO: 2, or an amino acid sequence having at least about 85%, at least about 90%, at least about 95%, at least about 96%, at least about 97%, at least about 98%, at least about 99%, or 100% sequence identity to the amino acid sequence of SEQ ID NO: 2.

29. The method of claim 27 or claim 28, wherein the ISG comprises the amino acid sequence of amino acid residues 155-207 of SEQ ID NO: 2, or of amino acid residues corresponding to amino acid residues 155-207 of an α/β-gliadin A-II polypeptide.

30. The method of claim 27 or claim 28, wherein the ISG comprises the amino acid sequence of SEQ ID NO: 8, or an amino acid sequence having at least about 85%, at least about 90%, at least about 95%, at least about 96%, at least about 97%, at least about 98%, at least about 99%, or 100% sequence identity to the amino acid sequence of SEQ ID NO: 8.

31. The method of claim 22, wherein the α/β-gliadin polypeptide is an α/β-gliadin A-III polypeptide.

32. The method of claim 31, wherein the α/β-gliadin A-III polypeptide comprises the amino acid sequence of SEQ ID NO: 3, or an amino acid sequence having at least about 85%, at least about 90%, at least about 95%, at least about 96%, at least about 97%, at least about 98%, at least about 99%, or 100% sequence identity to the amino acid sequence of SEQ ID NO: 3.

33. The method of claim 31 or claim 32, wherein the ISG comprises the amino acid sequence of amino acid residues 152-199 of SEQ ID NO: 3, or of amino acid residues corresponding to amino acid residues 152-199 of an α/β-gliadin A-III polypeptide.

34. The method of claim 31 or claim 32, wherein the ISG comprises the amino acid sequence of SEQ ID NO: 9, or an amino acid sequence having at least about 85%, at least about 90%, at least about 95%, at least about 96%, at least about 97%, at least about 98%, at least about 99%, or 100% sequence identity to the amino acid sequence of SEQ ID NO: 9.

35. The method of claim 22, wherein the α/β-gliadin polypeptide is an α/β-gliadin A-IV polypeptide.

36. The method of claim 35, wherein the α/β-gliadin A-IV polypeptide comprises the amino acid sequence of SEQ ID NO: 4, or an amino acid sequence having at least about 85%, at least about 90%, at least about 95%, at least about 96%, at least about 97%, at least about 98%, at least about 99%, or 100% sequence identity to the amino acid sequence of SEQ ID NO: 4.

37. The method of claim 35 or claim 36, wherein the ISG comprises the amino acid sequence of amino acid residues 167-215 of SEQ ID NO: 4, or of amino acid residues corresponding to amino acid residues 167-215 of an α/β-gliadin A-IV polypeptide.

38. The method of claim 35 or claim 36, wherein the ISG comprises the amino acid sequence of SEQ ID NO: 10, or an amino acid sequence having at least about 85%, at least about 90%, at least about 95%, at least about 96%, at least about 97%, at least about 98%, at least about 99%, or 100% sequence identity to the amino acid sequence of SEQ ID NO: 10.

39. The method of claim 22, wherein the α/β-gliadin polypeptide is an α/β-gliadin A-V polypeptide.

40. The method of claim 39, wherein the α/β-gliadin A-V polypeptide comprises the amino acid sequence of SEQ ID NO: 5, or an amino acid sequence having at least about 85%, at least about 90%, at least about 95%, at least about 96%, at least about 97%, at least about 98%, at least about 99%, or 100% sequence identity to the amino acid sequence of SEQ ID NO: 5.

41. The method of claim 39 or claim 40, wherein the ISG comprises the amino acid sequence of amino acid residues 164-223 of SEQ ID NO: 5, or of amino acid residues corresponding to amino acid residues 164-223 of an α/β-gliadin A-V polypeptide.

42. The method of claim 39 or claim 40, wherein the ISG comprises the amino acid sequence of SEQ ID NO: 11, or an amino acid sequence having at least about 85%, at least about 90%, at least about 95%, at least about 96%, at least about 97%, at least about 98%, at least about 99%, or 100% sequence identity to the amino acid sequence of SEQ ID NO: 11.

43. The method of claim 8, wherein the gliadin polypeptide is a γ-gliadin B-I polypeptide.

44. The method of claim 43, wherein the ISG comprises the amino acid sequence of SEQ ID NO: 19, or an amino acid sequence having at least about 85%, at least about 90%, at least about 95%, at least about 96%, at least about 97%, at least about 98%, at least about 99%, or 100% sequence identity to the amino acid sequence of SEQ ID NO: 19.

45. The method of claim 8, wherein the gliadin polypeptide is a γ-gliadin B-II polypeptide.

46. The method of claim 45, wherein the ISG comprises the amino acid sequence of SEQ ID NO: 20, or an amino acid sequence having at least about 85%, at least about 90%, at least about 95%, at least about 96%, at least about 97%, at least about 98%, at least about 99%, or 100% sequence identity to the amino acid sequence of SEQ ID NO: 20.

47. The method of claim 8, wherein the gliadin polypeptide is a γ-gliadin B-III polypeptide.

48. The method of claim 47, wherein the ISG comprises the amino acid sequence of SEQ ID NO: 21, or an amino acid sequence having at least about 85%, at least about 90%, at least about 95%, at least about 96%, at least about 97%, at least about 98%, at least about 99%, or 100% sequence identity to the amino acid sequence of SEQ ID NO: 21.

49. The method of claim 8, wherein the gluten polypeptide is a glutenin polypeptide.

50. The method of claim 49, wherein the glutenin polypeptide is a high molecular weight subunit 12 glutenin polypeptide.

51. The method of claim 50, wherein the glutenin polypeptide comprises the amino acid sequence of SEQ ID NO: 6, or an amino acid sequence having at least about 85%, at least about 90%, at least about 95%, at least about 96%, at least about 97%, at least about 98%, at least about 99%, or 100% sequence identity to the amino acid sequence of SEQ ID NO: 6.

52. The method of claim 50 or claim 51, wherein the ISG comprises the amino acid sequence of amino acid residues 45-86 of SEQ ID NO: 6, or of amino acid residues corresponding to amino acid residues 45-86 of a high molecular weight subunit 12 glutenin polypeptide.

53. The method of claim 50 or claim 51, wherein the ISG comprises the amino acid sequence of SEQ ID NO: 12, or an amino acid sequence having at least about 85%, at least about 90%, at least about 95%, at least about 96%, at least about 97%, at least about 98%, at least about 99%, or 100% sequence identity to the amino acid sequence of SEQ ID NO: 12.

54. The method of claim 8, wherein the gluten polypeptide is a hordein polypeptide.

55. The method of claim 54, wherein the hordein polypeptide is a γ-hordein-1 polypeptide.

56. The method of claim 55, wherein the γ-hordein-1 polypeptide comprises the amino acid sequence of SEQ ID NO: 13, or an amino acid sequence having at least about 85%, at least about 90%, at least about 95%, at least about 96%, at least about 97%, at least about 98%, at least about 99%, or 100% sequence identity to the amino acid sequence of SEQ ID NO: 13.

57. The method of claim 55 or claim 56, wherein the ISG comprises the amino acid sequence of amino acid residues 182-235 of SEQ ID NO: 13, or of amino acid residues corresponding to amino acid residues 182-235 of a γ-hordein-1 polypeptide.

58. The method of claim 55 or claim 56, wherein the ISG comprises the amino acid sequence of SEQ ID NO: 24, or an amino acid sequence having at least about 85%, at least about 90%, at least about 95%, at least about 96%, at least about 97%, at least about 98%, at least about 99%, or 100% sequence identity to the amino acid sequence of SEQ ID NO: 24.

59. The method of claim 54, wherein the hordein polypeptide is a γ-hordein-3 polypeptide.

60. The method of claim 59, wherein the γ-hordein-3 polypeptide comprises the amino acid sequence of SEQ ID NO: 14, or an amino acid sequence having at least about 85%, at least about 90%, at least about 95%, at least about 96%, at least about 97%, at least about 98%, at least about 99%, or 100% sequence identity to the amino acid sequence of SEQ ID NO: 14.

61. The method of claim 59 or claim 60, wherein the ISG comprises the amino acid sequence of amino acid residues 165-214 of SEQ ID NO: 14, or of amino acid residues corresponding to amino acid residues 165-214 of a γ-hordein-3 polypeptide.

62. The method of claim 59 or claim 60, wherein the ISG comprises the amino acid sequence of SEQ ID NO: 25, or an amino acid sequence having at least about 85%, at least about 90%, at least about 95%, at least about 96%, at least about 97%, at least about 98%, at least about 99%, or 100% sequence identity to the amino acid sequence of SEQ ID NO: 25.

63. The method of claim 54, wherein the hordein polypeptide is a B1-hordein polypeptide.

64. The method of claim 63, wherein the B1-hordein polypeptide comprises the amino acid sequence of SEQ ID NO: 15, or an amino acid sequence having at least about 85%, at least about 90%, at least about 95%, at least about 96%, at least about 97%, at least about 98%, at least about 99%, or 100% sequence identity to the amino acid sequence of SEQ ID NO: 15.

65. The method of claim 63 or claim 64, wherein the ISG comprises the amino acid sequence of amino acid residues 160-208 of SEQ ID NO: 15, or of amino acid residues corresponding to amino acid residues 160-208 of a B1-hordein polypeptide.

66. The method of claim 63 or claim 64, wherein the ISG comprises the amino acid sequence of SEQ ID NO: 22, or an amino acid sequence having at least about 85%, at least about 90%, at least about 95%, at least about 96%, at least about 97%, at least about 98%, at least about 99%, or 100% sequence identity to the amino acid sequence of SEQ ID NO: 22.

67. The method of claim 54, wherein the hordein polypeptide is a B3-hordein polypeptide.

68. The method of claim 67, wherein the ISG comprises the amino acid sequence of SEQ ID NO: 23, or an amino acid sequence having at least about 85%, at least about 90%, at least about 95%, at least about 96%, at least about 97%, at least about 98%, at least about 99%, or 100% sequence identity to the amino acid sequence of SEQ ID NO: 23.

69. The method of claim 8, wherein the gluten polypeptide is a secalin polypeptide.

70. The method of claim 69, wherein the secalin polypeptide is a 75 k gamma secalin polypeptide.

71. The method of claim 70, wherein the 75 k gamma secalin polypeptide comprises the amino acid sequence of SEQ ID NO: 16, or an amino acid sequence having at least about 85%, at least about 90%, at least about 95%, at least about 96%, at least about 97%, at least about 98%, at least about 99%, or 100% sequence identity to the amino acid sequence of SEQ ID NO: 16.

72. The method of claim 8, wherein the gluten polypeptide is an avenin polypeptide.

73. The method of claim 72, wherein the avenin polypeptide is an avenin-3 polypeptide.

74. The method of claim 73, wherein the avenin-3 polypeptide comprises the amino acid sequence of SEQ ID NO: 17, or an amino acid sequence having at least about 85%, at least about 90%, at least about 95%, at least about 96%, at least about 97%, at least about 98%, at least about 99%, or 100% sequence identity to the amino acid sequence of SEQ ID NO: 17.

75. The method of claim 73 or claim 74, wherein the ISG comprises the amino acid sequence of amino acid residues 85-141 of SEQ ID NO: 17, or of amino acid residues corresponding to amino acid residues 85-141 of an avenin-3 polypeptide.

76. The method of claim 73 or claim 74, wherein the ISG comprises the amino acid sequence of SEQ ID NO: 27, or an amino acid sequence having at least about 85%, at least about 90%, at least about 95%, at least about 96%, at least about 97%, at least about 98%, at least about 99%, or 100% sequence identity to the amino acid sequence of SEQ ID NO: 27.

77. The method of claim 72, wherein the avenin polypeptide is an avenin-E polypeptide.

78. The method of claim 77, wherein the avenin-E polypeptide comprises the amino acid sequence of SEQ ID NO: 18, or an amino acid sequence having at least about 85%, at least about 90%, at least about 95%, at least about 96%, at least about 97%, at least about 98%, at least about 99%, or 100% sequence identity to the amino acid sequence of SEQ ID NO: 18.

79. The method of claim 77 or claim 78, wherein the ISG comprises the amino acid sequence of amino acid residues 71-130 of SEQ ID NO: 18, or of amino acid residues corresponding to amino acid residues 71-130 of an avenin-E polypeptide.

80. The method of claim 77 or claim 78, wherein the ISG comprises the amino acid sequence of SEQ ID NO: 26, or an amino acid sequence having at least about 85%, at least about 90%, at least about 95%, at least about 96%, at least about 97%, at least about 98%, at least about 99%, or 100% sequence identity to the amino acid sequence of SEQ ID NO: 26.

81. The method of any one of claims 1-80, wherein the gluten polypeptide with a reduced inflammatory potential comprises:
(a) a reduced ability to remodel a cell membrane;
(b) a reduced ability to access an endosomal compartment within a cell;
(c) a reduced ability to mediate organization of innate immune ligands into ordered nanocrystalline structures;
(d) a reduced ability to promote activation of Toll-Like Receptors (TLRs); and/or
(e) a reduced ability to self-assemble into amyloidal or protofibril structures to activate formyl peptide receptor-like 1 (FPRL1) and/or formyl peptide receptor 2 (FPR2),
as compared to a gluten polypeptide without the one or more alterations in the ISG.

82. The method of claim 81, wherein the reduced ability to remodel a cell membrane comprises a reduced ability to permeabilize a cell membrane, as compared to a gluten polypeptide without the one or more alterations in the ISG.

83. The method of claim 81, wherein the innate immune ligands comprise one or more nucleic acid molecules.

84. The method of claim 83, wherein the one or more nucleic acid molecules comprise one or more double-stranded DNA (dsDNA), double-stranded RNA (dsRNA), single-stranded DNA (ssDNA), and/or single-stranded RNA (ssRNA) molecules.

85. An isolated nucleic acid molecule comprising a nucleotide sequence encoding a gluten polypeptide with a reduced inflammatory potential produced by the method of any one of claims 1 and 7-84.

86. A vector comprising the isolated nucleic acid molecule of claim 85.

87. A host cell comprising the vector of claim 86.

88. A plant, plant tissue, plant organ, plant part, or plant cell comprising the nucleic acid of claim 85 or the vector of claim 86.

89. A plant, plant tissue, plant organ, plant part, or plant cell comprising a gluten polypeptide with a reduced inflammatory potential produced by the method of any one of claims 1 and 7-84.

90. A modified plant, plant tissue, plant organ, plant part, or plant cell produced by the method of any one of claims 2-84.

91. The plant, plant tissue, plant organ, plant part, or plant cell of any one of claims 88-90, wherein the plant, plant tissue, plant organ, plant part, or plant cell is a wheat, rye, barley or oat plant, plant tissue, plant organ, plant part, or plant cell.

92. An isolated gluten polypeptide with a reduced inflammatory potential produced by the method of any one of claims 1 and 7-84.

93. A composition comprising the gluten polypeptide of claim 92.

94. A composition comprising the plant, plant tissue, plant organ, plant part, or plant cell of any one of claims 88-91.

95. The composition of claim 93 or claim 94, wherein the composition is an orally consumable composition, a foodstuff composition, a beverage product, a nutraceutical composition, a dietary supplement, an edible gel, a pharmaceutical composition, or a cosmetic or skin composition.
